(19) United States
(12) Patent Application Publication
(10) Pub. No.: US 2024/0226191 A1
(43) Pub. Date: Jul. 11, 2024

(54) COMPOSITIONS AND METHODS FOR TREATING DISEASE

(71) Applicant: MICROBA IP PTY LTD, Brisbane City, Queensland (AU)

(72) Inventors: Páraic Ó CUÍV, Brisbane (AU); Lutz KRAUSE, Brisbane (AU)

(21) Appl. No.: 18/560,020

(22) PCT Filed: May 10, 2022
(86) PCT No.: PCT/AU2022/050442

§ 371 Date: Nov. 9, 2023

(30) Foreign Application Priority Data
May 10, 2021 (AU) 2021901387
May 6, 2022 (AU) 2022901200

(51) Int. Cl. A61K 35/74; A61K 35/00; A61P 1/04; A61P 29/00; A61P 37/02; C12N 1/20

(52) U.S. Cl. CPC A61K 35/74; A61P 1/04; A61P 29/00; A61P 37/02; C12N 1/205; A61K 2035/11

(57) ABSTRACT

This disclosure relates generally to the field of therapeutic compositions comprising bacterial strains and methods for the treatment or prevention of disease. More particularly, the disclosure relates to compositions comprising bacterial strains isolated from the human digestive tract and their use in the treatment or prevention of inflammatory and autoimmune disorders.

### RELATED APPLICATIONS

This invention claims priority to Australian Provisional Application Nos. 2021901387 entitled “Compositions and Methods for Treating Disease” filed 10 May 2021, and 2022901200 entitled “Compositions and Methods for Treating Disease” filed 6 May 2022, the entire contents of which are incorporated herein by reference.

### FIELD OF THE INVENTION

This invention relates generally to the field of therapeutic compositions comprising bacterial strains and methods for the treatment or prevention of disease. More particularly, the present invention relates to compositions comprising bacterial strains isolated from the human digestive tract and their use in the treatment or prevention of inflammatory and autoimmune disorders.

### BACKGROUND OF THE INVENTION

The human gut microbiota contains more than 500-1000 different phylotypes belonging to a few bacterial phylum, including Firmicutes, Bacteroidetes, Proteobacteria, Fusobacteria, and Verrucomicrobia. The two major phyla, the Bacteroidetes and the Firmicutes, represent over 90% of the gut microbiota (Arumugam et al., 2011). The successful symbiotic relationships arising from bacterial colonisation of the human gut have yielded a wide variety of metabolic, structural, protective and other beneficial functions. The gut bacteria are key regulators of digestion along the gastrointestinal (GI) tract; with commensal bacterial playing an important role in the extraction, synthesis, and absorption of many nutrients and metabolites, including bile acids, lipids, amino acids, vitamins, and short-chain fatty acids (SCFAs). More recently, the immunological importance of the gut microbiota and their products in regulating the development, homeostasis, and function of innate and adaptive immune cells have been recognised (Brestoff and Atris, 2013).

It is now increasingly recognised that the gut microbiome regulates host intestinal mucosal immunity and predisposition to inflammation (Geva-Zatorsky et al., 2017; Kabat et al., 2014), opening new avenues for novel therapeutic interventions.

Dramatic changes in microbiota composition have been documented in many inflammatory and autoimmune disorders, including inflammatory bowel disease (IBD). In recognition of the potential positive effect that certain bacterial strains may have on the animal gut, various strains have been proposed for use in the treatment of various diseases. Certain strains, including Lactobacillus and Bifidobacterium strains, have been proposed for use in treating various extraintestinal inflammatory and autoimmune disorders (see, Goldin & Gorbach, 2008; Azad et al., 2013). However, the precise effects of particular bacterial strains locally at the GI tract and systemically throughout the body are unresolved. As a result, the relationships between different diseases and different bacterial strains in the human GI tract are yet to be clearly elucidated.

IBD (including the two major disease subtypes Crohn's disease (CD) and ulcerative colitis (UC)) is characterised by episodic and disabling inflammation of the GI tract. In 2017, it is estimated that 6.8 million people globally suffered from IBD, with the highest prevalence in the United States and Europe (GBD 2017; Inflammatory Bowel Disease Collaborators, 2019). Up to 20% of patients are diagnosed before the age of 16 and paediatric-onset IBD (PIBD) is associated with a more complicated and aggressive disease with adverse impacts on growth and psychosocial development.

There is currently no cure for IBD, and long-term clinical management requires effective therapeutics with an excellent safety profile. However, existing treatments show a range of deficiencies and remission is generally short. Moreover, PIBD therapeutics are ineffective where early onset coupled with more aggressive disease result in progressive bowel damage and need for surgery. There is an urgent need to develop more effective and safe therapies to improve patient quality of life, maintain remission over long periods, reduce surgery and curtail individual and public health costs.

Existing treatments for IBD are sub-optimal with strong adverse effects, low compliance (50% average non-adherence rates (see, Chan et al., 2017)) and high cost. Furthermore, there is no effective solution to maintaining extended periods of disease-free remission. Mesalmine, one of the most widely used first line therapies for mild to moderate flares of ulcerative colitis and for maintenance of remission, has response rates between 40%-70% and remission rates of 15%-20% (Karagozian & Burakoff, 2007).

There is a requirement in the art for new methods of treating inflammatory and autoimmune disorders. There is also a requirement for the potential effects of gut bacteria to be characterised so that new therapies using gut bacteria can be developed.

### SUMMARY OF THE INVENTION

The present invention is predicated in part on the inventors' identification that bacterial strains of Mediterraneibacter faecis enhance or improve gut barrier function. Based on this consideration, it is proposed that strains of M. faecis are particularly suited to therapeutic applications for treating and preventing inflammatory and autoimmune disorders, as described hereinafter.

The inventors have developed new compositions comprising a viable bacterial strain of the species Mediterraneibacter faecis that can be used for treating and preventing inflammatory and autoimmune disorders.

Accordingly, in one aspect the invention provides a cell of the Mediterraneibacter faecis strains deposited under any one of accession numbers V21/006223, V21/006224, V21/006225, or V21/006226, or a derivative thereof.

In some embodiments, the cell is at least partially isolated.

In another aspect, the invention provides a biologically pure culture of the Mediterraneibacter faecis strain deposited under any one of accession numbers V21/006223, V21/006224, V21/006225, or V21/006226, or a derivative thereof.

In another aspect, the present invention provides a composition comprising the cell or culture as described above and elsewhere herein.

In yet another aspect, the present invention provides a composition comprising a bacterial strain with a 16S rRNA sequence that is at least about 97.5%, 98%, 98.5% 99%, 99.1%, 99.2%, 99.3%, 99.4%, 99.5%, 99.6%, 99.7%, 99.8%, or 99.9%, identical to any one of SEQ ID NOs: 1-24; or with a 16S rRNA gene sequence represented by any one of SEQ ID NOs: 1-24. In some embodiments, the bacterial strain comprises two or more copies (e.g., two copies, three copies, four copies, five copies, six copies, seven copies, eight copies) of a 16S rRNA sequence independently selected form the 16S rRNA sequences set forth in SEQ ID Nos: 1-24.

In some embodiments, the composition further comprises a pharmaceutically acceptable excipient, diluent, or carrier.

In yet another aspect, the present invention provides a pharmaceutical composition comprising a bacterial strain with a 16S rRNA sequence that is at least about 97%, 98%, 99%, 99.5%, 99.6%, 99.7%, 99.8%, or 99.9% identical to the 16S rRNA sequence of a bacterial strain of the Mediterraneibacter faecis, together with a pharmaceutically acceptable carrier, diluent, or excipient.

In a related aspect, the present invention provides a pharmaceutical composition comprising an effective amount of a bacterial strain that is a phylogenetic descendant of the most recent common ancestor (MRCA) of M. faecis and M. lactaris, together with a pharmaceutically acceptable carrier, diluent, or excipient. Suitably, the MRCA is defined at node 52630 of the bac120 phylogenetic tree from r95 of the Genome Taxonomy Database (GTDB). In some embodiments, the phylogenetic tree is created by release 95 of the GTDB, however, any suitable subsequent release is considered to give equally as applicable results. In some preferred embodiments of this type, the bacterial strain is of M. faecis. In some alternative embodiments, the bacterial strain is of M. lactaris.

Typically, the bacterial strain is at least partially isolated.

In some embodiments, the bacterial strain is live. In some alternative embodiments, the bacterial strain is dead.

In some embodiments, the compositions further comprise a prebiotic.

In some embodiments, the composition is formulated in a dried form. Typically, the composition is dried using techniques selected from lyophilization, spray drying, fluidized bed drying, vacuum drying, or a combination thereof.

In some embodiments, the composition is formulated for oral administration.

In some embodiments, the bacterial strain produces an agent that attenuates or impairs signal transducer and activator of transcription 3 (STAT3) signalling in a cell.

In some embodiments of this type, the agent is a small molecule, peptide, or nucleotide. Typically, the agent is released by the bacterial strain.

In some embodiments, the agent binds specifically to any one of STAT3, JAK2, TYK, or IL-23.

In some embodiments, the M. faecis strain produces one or more metabolites selected from the group comprising or consisting of propionate, lactate, acetate, and formate. In some of the same embodiments and some other embodiments, the M. faecis strain does not produce butyrate.

In some embodiments, the M. faecis strain produces vitamin B12.

In another aspect, the present invention provides a method of restoring or improving gut barrier function in a subject, the method comprising administering to the subject a bacterial strain of the species Mediterraneibacter faecis, to thereby restore or improve gut barrier function.

In some preferred embodiments, restoring or improving gut barrier function is characterised by at least one of: (i) an increase in the quality and/or quantity of mucin; (ii) improvement in integrity of tight junction proteins; (iii) reduction in translocation of luminal contents into systemic circulation; or (iv) reduction of intestinal ulcers and/or intestinal wounds.

In some embodiments, the luminal contents includes lipopolysaccharide (LPS).

In some embodiments, the restoration or improvement in gut barrier function results in a reduction in systemic inflammation in the subject. In some embodiments of this type, the systemic inflammation is characterized by elevated levels of an inflammatory cytokine (e.g., IL-1β IL-8, IL-6, and TNF) in the subject as compared to the level of the inflammatory cytokine in a healthy subject.

In yet another aspect, the present invention provides a method of maintaining gut barrier function in a subject, the method comprising administering to the subject a bacterial strain of the species Mediterraneibacter faecis, to thereby maintain gut barrier function in the subject.

In yet another aspect, the present invention provides a method of reducing inflammation in a subject, the method comprising administering to the subject a bacterial strain of the strain Mediterraneibacter faecis, to thereby reduce inflammation in the subject.

In some embodiments, the inflammation is local to the gut environment, or systemic inflammation.

In another aspect, the present invention provides a method of inducing or enhancing mucosal healing in a subject, the method comprising administering to the subject a bacterial strain of the species Mediterraneibacter faecis in an amount sufficient to induce epithelial cell migration, proliferation and/or differentiation, to thereby induce mucosal healing in the subject.

In some embodiments, mucosal healing in the subject can be measured using one or more fecal or serum markers. By way of an illustrative example, one or more fecal markers may be selected from the group comprising calprotectin, lactoferrin, metalloproteinase (MMP)-9, and lipocalin-2.

In some embodiments, the bacterial strain reduces inflammation by attenuating the NFκB pathway. In some embodiments of this type, the bacterial strain inhibits the production of one or more transcription factors, cytokines, or chemokines selected from the group comprising NFκB, TNF, IFN-γ, IL-1β, IL-8, and MCP-1.

In yet another aspect, the present invention provides methods of blocking or otherwise inhibiting STAT3 signalling in a target cell, the method comprising contacting the cell with at least a soluble component of a bacterial cell preparation of the species Mediterraneibacter faecis, to block or otherwise inhibit STAT3 signalling in the cell. Typically, the method of this aspect is performed in vitro.

In some embodiments, the target cell is selected from a reporter cell (e.g., a HEK cell), an immune cell (e.g., a Th17 immune cell), an epithelial cell, or an endothelial cell. In some embodiments, the target cell is a mammalian cell, and preferably, a human cell.

In some embodiments, the bacterial cell preparation is a bacterial cell culture. The soluble component may therefore comprise, consist, or consist essentially of, the soluble fraction of the bacterial cell culture (e.g., the cell culture supernatant). The soluble component may further comprise some insoluble components of the bacterial cell culture. For example, the soluble component may include substantially all of the bacterial culture. Preferably, the soluble component is substantially depleted of bacterial cells.

In some alternative embodiments, the bacterial cell preparation is a bacterial cell lysate. In exemplary embodiments of this type, the soluble component may relate to the soluble fraction of the cell lysate. A soluble fraction can suitably be achieved by any method, including by centrifugation.

In still yet another aspect, the present invention provides a method of blocking or otherwise inhibiting STAT3 signalling in a cell, the method comprising administering a bacterial strain of the species Mediterraneibacter faecis to the subject, thereby blocking or otherwise inhibiting STAT3 signalling in the cell. Typically, the methods of this aspect are performed in vivo.

In some embodiments, the cell is an immune cell (e.g., a Th17 immune cell) or epithelial cell.

In some embodiments, the cell is an epithelial cell, and the bacterial strain or a metabolite produced by the bacterial strain increases the production of IL-22 in the subject.

In some embodiments, the bacterial strain produces a molecule that is a direct inhibitor or an indirect inhibitor of STAT3. For example, the bacterial strain may produce a metabolite that directly inhibits at least one of an IL-23 polypeptide, a JAK2 polypeptide, a TYK2 polypeptide, or a STAT3 polypeptide.

In some embodiments, the bacterial strains used in the methods described above and elsewhere herein produce one or more metabolites selected from propionate, lactate, acetate, and formate. In some of the same embodiments, and some alternative embodiments, the bacterial strain produces vitamin B12. In some embodiments, the bacterial strain does not produce butyrate.

In some embodiments the bacterial strain has a 16S rRNA sequence that is at least about 97.5%, 98.5%, 99%, 99.1%, 99.2%, 99.3%, 99.4%, 99.5%, 99.6%, 99.7%, 99.8%, or 99.9% identical to the 16S rRNA sequence of a bacterial strain of M. faecis.

In some alternative embodiments, the bacterial strain has a 16S rRNA sequence that is at least about 97.5%, 98.5%, 99%, 99.1%, 99.2%, 99.3%, 99.4%, 99.5%, 99.6%, 99.7%, 99.8%, or 99.9% identical to any one of SEQ ID NOs: 1-24, or when the bacterial strain has the 16S rRNA gene sequence represented by any one of SEQ ID NOs: 1-24. In some embodiments, the bacterial strain comprises two or more copies (for example, two copies, three copies, four copies, five copies, six copies, seven copies, eight copies) of a 16S rRNA sequence independently selected from the 16S rRNA sequences set forth in SEQ ID NO: 1-24.

In some embodiments the bacterial strain has a 16S rRNA sequence that is at least about 97.5%, 98.5%, 99%, 99.1%, 99.2%, 99.3%, 99.4%, 99.5%, 99.6%, 99.7%, 99.8%, or 99.9% identical to the 16S rRNA sequence of a bacterial strain of M. lactaris.

In some alternative embodiments, the bacterial strain has a 16S rRNA sequence that is at least about 97.5%, 98.5%, 99%, 99.1%, 99.2%, 99.3%, 99.4%, 99.5%, 99.6%, 99.7%, 99.8%, or 99.9% identical to any one or more of SEQ ID NOs: 29-32, or when the bacterial strain has the 16S rRNA gene sequence represented by any one or more of SEQ ID NOs: 29-32.

In some embodiments, the bacterial strain is the M. faecis strain deposited under any one of accession numbers V21/006223, V21/006224, V21/006225, or V21/006226, or a derivative thereof.

Preferably, the bacterial strain is at least partially isolated.

In some embodiments, the bacterial strain is formulated as a pharmaceutical composition, further comprising a pharmaceutically acceptable carrier, diluent or excipient. In some embodiments, the pharmaceutical composition is a dry composition. In some embodiments, the dry composition is selected from the group consisting of particles, granules, and powder. By way of an illustrative example the pharmaceutical composition may be lyophilized, spray dried, fluidized bed dried, vacuum dried, or a combination thereof.

In some embodiments, the pharmaceutical composition is formulated for oral administration.

In still yet other aspects, the present invention provides a method of treating an inflammatory or autoimmune disorder in a subject, the method comprising administering an effective amount of a bacterial strain of Mediterraneibacter faecis to the subject, to thereby treat or prevent the inflammatory or autoimmune disorder.

In some embodiments, the inflammatory or autoimmune disorder is selected from the group comprising an inflammatory bowel disease (such as Crohn's disease or ulcerative colitis); asthma (such as allergic asthma or neutrophilic asthma); arthritis (such as rheumatoid arthritis, osteoarthritis, psoriatic arthritis, or juvenile idiopathic arthritis); fatty liver disease (such as nonalcoholic fatty liver disease (NAFLD)); ankylosing spondylitis; psoriasis; systemic lupus erythematosus (SLE); scleroderma; Sjogren's syndrome; vasculitis; type 1 diabetes mellitus. Preferably, the inflammatory or autoimmune disorder is an inflammatory bowel disease (IBD).

In some embodiments, the bacterial strain blocks or otherwise inhibits STAT3 signalling in at least a cell of the subject. Typically, the cell is an epithelial cell, endothelial cell or an immune cell (e.g., a Th17 immune cell).

In some embodiments, the bacterial strain produces one or more metabolites selected from the group comprising propionate, lactate, acetate, and formate. In some of the same embodiments, and some other embodiments, the bacterial strain does not produce butyrate.

In some embodiments, the bacterial strain produces vitamin B12.

In some embodiments, the bacterial strain has a 16S rRNA sequence that is at least about 95%, 96%, 97%, 98%, 99%, 99.5% or 99.9% identical to the 16S rRNA sequence of a bacterial strain of the genus Mediterraneibacter.

Alternatively, in some embodiments the bacterial strain has a 16S rRNA sequence that is at least about 95%, 96%, 97%, 98%, 99%, 99.5% or 99.9% identical to SEQ ID NO: 1-24, or when the bacterial strain has the 16S rRNA sequence represented by any one of SEQ ID NOs: 1-24.

Alternatively, in some embodiments the bacterial strain has a 16S rRNA sequence that is at least about 95%, 96%, 97%, 98%, 99%, 99.5% or 99.9% identical to any one or more of SEQ ID NOs: 29-32, or when the bacterial strain has the 16S rRNA sequence represented by any one or more of SEQ ID NOs: 29-32.

Preferably, the bacterial strain is at least partially isolated.

In some embodiments, the bacterial strain is formulated as a pharmaceutical composition, together with a pharmaceutically acceptable carrier, diluent, and/or excipient. In some embodiments, the composition is a dry composition selected from the group consisting of particles, granules, and powder. For example, the composition may be lyophilized. Alternatively, the composition may be spray dried, fluidized bed dried, or vacuum dried.

In some embodiments, the composition is formulated for oral administration.

In one aspect, the present invention provides a composition comprising a bacterial strain of the genus Mediterraneibacter for use in therapy.

In another aspect, the present invention provides a composition comprising a bacterial strain of Mediterraneibacter faecis, for use in therapy.

In another aspect, the present invention provides a composition comprising a bacterial strain of Mediterraneibacter lactaris for use in therapy.

In yet another aspect, the present invention provides a composition comprising a bacterial strain of the genus Mediterraneibacter, for use in the treatment or prevention of an inflammatory or autoimmune disorder.

In still yet another aspect, the present invention provides a composition comprising a bacterial strain of Mediterraneibacter faecis, for use in the treatment or prevention of an inflammatory or autoimmune disorder.

In some embodiments, the bacterial strain is the M. faecis strain deposited under any one of accession numbers V21/006223, V21/006224, V21/006225, or V21/006226, or a derivative thereof.

In a related aspect, the present invention provides a composition comprising a bacterial strain of Mediterraneibacter lactaris, for use in the treatment or prevention of an inflammatory or autoimmune disorder.

In some embodiments, the inflammatory or autoimmune disorder is selected from an inflammatory bowel disease (such as Crohn's disease or ulcerative colitis); asthma (such as allergic asthma or neutrophilic asthma); arthritis (such as rheumatoid arthritis, osteoarthritis, psoriatic arthritis, or juvenile idiopathic arthritis); fatty liver disease (such as nonalcoholic fatty liver disease (NAFLD)); ankylosing spondylitis; psoriasis; systemic lupus erythematosus (SLE); scleroderma; Sjogren's syndrome; vasculitis; type 1 diabetes mellitus. In some preferred embodiments, the inflammatory or autoimmune disorder is an inflammatory bowel disease (IBD).

In one aspect, the present invention provides a composition for use in treating an inflammatory or autoimmune disorder, the composition comprising a bacterial strain of Mediterraneibacter faecis; and an anti-inflammatory agent.

In some embodiments, the anti-inflammatory agent is selected from the group comprising 5-aminosalicylates, corticosteroids, azathioprine, infliximab, and adalimumab.

In another aspect, the present invention provides a composition for use in treating an inflammatory or autoimmune disorder, the composition comprises a bacterial strain of Mediterraneibacter faecis; and a nutritional supplement. In embodiments of this type, the nutritional supplement improves engraftment of the bacterial stain.

In some related aspects, the technology described herein provides bacterial species and compositions comprising them in for form of probiotics. Preferably, such probiotics are effective to improve intestinal microbial ecology, alleviate symptoms of microbial dysbiosis, promote wellness, and/or treat or prevent inflammatory and/or autoimmune disorders.

### BRIEF DESCRIPTION OF THE FIGURES

The following figures form part of the present specification and are included to further demonstrate certain aspects of the present disclosure. The disclosure may be better understood by reference to one or more of these figures in combination with the detailed description of specific embodiments presented herein.

FIG. 1 provides a graphical phylogenetic tree that displays a focused view of node 52630 of the bac120 phylogenetic tree from the GTDB. The GTDB tree is a genome tree constructed from a concatenated alignment of 120 conserved single-copy bacterial marker genes (Parks et al. 2018). The Most Recent Common Ancestor (MRCA) of M. lactaris and M. faecis (node 52630) is highlighted.

FIG. 2 provides a graphical representation of bacterial strain associations with a broad range of inflammatory and autoimmune disorders. Using high resolution gut metagenomic data of 6,020 subjects, we identified (A) M. faecis and (B) M. lactaris as being prevalent in healthy humans (dark bar) but depleted in a range of medical conditions (striped bars). All shown association have a FDR <0.01 (Fisher's exact test).

FIG. 3 provides graphical representation of the phylogeny, metabolism, and morphology of M. faecis strains. (A) A genome tree constructed with an alignment of 120 bacteria-specific single copy marker genes from high quality reference genomes from the GTDB (release 95). Non-parametric bootstrap values calculated from 1000 iterations. (B) Gram-staining of MH-23 isolate MH23-1 showing morphology.

FIG. 4 provides graphical representations of the effect of M. faecis on naïve animals. (A) Overview of the model used to assess the effect of M. faecis MH23-1 on naïve C57Bl/6 mice. (B) Treatment with M. faecis MH23-1 has little effect on body weight of naïve animals. (C)-(D) Treatment with M. faecis MH23-1 has no effect on colon length or colon weight/length ratio relative to vehicle treated controls in naïve animals. (E)-(G) Treatment with M. faecis MH23-1 has no effect on epithelial injury, inflammation or hypervascularization relative to vehicle treated controls in naïve animals. (H) Treatment with M. faecis MH23-1 has no effect on gut histology relative to vehicle treated controls in naïve animals. All data reported as mean and standard deviation. ns, not significant; * p<0.05.

FIG. 5 provides graphical representations that M. faecis MH23-1 restores gut barrier function. (A) Overview of the DSS model used to assess the therapeutic efficacy of M. faecis MH23-1. (B) Effect of daily treatment of vehicle, prednisone, F. prausnitzii A2-165 and M. faecis in health and DSS treated samples. All treatment groups were compared to the DSS+vehicle group. Significance was determined using a two-way Anova with Dunnett's test for multiple comparison. (C) Endoscopic assessment of colitis as assessed on days 1, 2 and 6. All groups were compared to the DSS+vehicle group for each individual day using a Kruskal-Wallis test with Dunn's correction for multiple comparisons (day 1) or one-way Anova with Dunnett's correction for multiple comparison (day 2, 6) as appropriate. All data presented as mean and standard deviation. (D) Representative gut histology images of C57Bl/6 mice treated with vehicle, prednisone, or M. faecis MH23-1. (E) DSS treatment results in an increase in the histopathological score that is ameliorated by treatment with prednisone F. prausnitzii A2-165 and M. faecis MH23-1. All data presented as mean and standard deviation. All groups were compared to the DSS+vehicle group and significance was determined using a one-way Anova with Dunnett's test for multiple comparison. (F) DSS treatment results in an increase in epithelial injury that is ameliorated by treatment with prednisone or M. faecis MH23-1. All data presented as mean and standard deviation. All groups were compared to the DSS+vehicle group using a Kruskal-Wallis test with Dunn's correction for multiple comparisons. (G) DSS treatment results in an increase in inflammation score that is ameliorated by treatment with prednisone or M. faecis MH23-1. All data presented as mean and standard deviation. All groups were compared to the DSS+vehicle group using a one-way Anova with Dunnett's correction for multiple comparisons. (H) Lipocalin-2 concentration in faeces of C57Bl/6 mice treated with vehicle, prednisone, F. prausnitzii or M. faecis MH23-1. Significance was determined using a one-way Anova with Dunnett's test for multiple comparison. (I) DSS treatment results in a significant decrease in the number of goblet cells relative to enterocytes and this is ameliorated by treatment with M. faecis MH23-1. All data presented as mean and standard deviation. All groups were compared to the DSS+vehicle group using a Kruskal-Wallis test with Dunnett's correction for multiple comparisons. (J) DSS treatment results in a significant decrease in the ratio of alcian blue staining that is ameliorated by treatment with M. faecis MH23-1 but not prednisone nor F. prausnitzii A2-165. All data presented as mean and standard deviation. All groups were compared to the DSS+vehicle group using Brown-Forsythe and Welch ANOVA tests with Dunnett's T3 correction for multiple comparisons. (ns: not significant; *, p<0.05; **, p<0.01; ****, p<0.0001). (K) Overview of the DSS model used to assess the therapeutic efficacy of M. faecis MH23-3. (L) Endoscopic assessment of colitis with all groups compared to the DSS+vehicle group. All data presented as mean and standard deviation. (M) DSS treatment results in an increase in the histopathological score that is ameliorated by treatment with prednisone and M. faecis MH23-3. All data presented as mean and standard deviation and all groups were compared to the DSS+vehicle group. (N) DSS treatment results in an increase in epithelial injury that is ameliorated by treatment with prednisone or M. faecis MH23-3. All data presented as mean and standard deviation and all groups were compared to the DSS+vehicle group. (0) DSS treatment results in an increase in inflammation score that is ameliorated by treatment with prednisone or MH23-3. All data presented as mean and standard deviation and all groups were compared to the DSS+vehicle group (P) Lipocalin-2 concentration in faeces of C57Bl/6 mice±DSS treated with vehicle, prednisone or M. faecis MH23-3. All data presented as mean and standard deviation and all groups were compared to the DSS+vehicle group. (Q) Overview of the TNBS model used to assess the therapeutic efficacy of M. faecis MH23-3. (R) DSS treatment results in an increase in the histopathological score that is ameliorated by treatment with prednisone and M. faecis MH23-3. All data presented as mean and standard deviation and all groups were compared to the DSS+vehicle group. (S) Representative gut histology images of C57Bl/6 mice±TNBS treated with vehicle, prednisone, or M. faecis MH23-1. (ns: not significant; *, p<0.05; **, p<0.01; ***, p<0.001; ****, p<0.0001).

FIG. 6 provides a graphical representation of M. faecis suppressing STAT3 and NF-kB activation in vitro. (A)-(C) STAT3 signalling is inhibited when HEKBlue IL-23 reporter cell lines are treated with cell-free supernatant of M. faecis MH23-1, MH23-3 and MH23-4 (t-test, n=3, (A) p<0.0001; (B) p<0.0001; (C) p=0.002). (D)-(F) Cell-free supernatant of M. faecis strains MH23-1, MH23-3 and MH23-4 were size fractionated. The <3 KDa fractions where heat treated at 37° C. or 97° C. then tested HEKBlue IL-23 reporter cell lines. After heat treatment, all fractions are still able to inhibit STAT3 signalling (t-test, n=6; (D) p=0.0068, p=0.0186, p=0.0005; (E) p=0.0008, p=0.0021, p=0.0002; (F) p<0.0001, p<0.0001, p<0.0001).

FIG. 7 provides a graphical representation of cytokine expression in guyt epithelial cells. (A) M. faecis MH23-1 culture supernatant suppresses IL-1p mediated IL-8 secretion in HCT116 gut epithelial cells. (B) After PMA-dependent maturation of THP-1 into active macrophages, M. faecis MH23-4 and MR1 supernatant reduce the level of (A) IL-8 and (B) TNF production when co-stimulated with LPS. Accordingly, TNF levels increase when the cells are treated with a bacteria associated to IBD such as C. bolteae (ANOVA test, n=3; p=0.0026). All data presented as mean and standard deviation.

FIG. 8 shows heatmaps of GPCR hits of MH23. Heatmaps of agonist (blue) and antagonist (green) hits for M. faecis MH23-1. Legend on the right side indicate the percentage of activation for each hit.

FIG. 9 provides a graphical representation of M. faecis modulates IL-22 and IFNγ in hPBMC-derived CD3⁺ and CD3⁻ cells. (A) M. faecis MH23-1 and MH23-2 cell-free supernatant treatment induces an increase of T cells only in unstimulated PBMC (ANOVA test, n=6, Untreated p=0.0010, +PIM p=0.0937). (B)-(C) M. faecis MH23-1 and MH23-2 cell-free supernatant treatment does not induce an increase of NK cells (B) ANOVA test, n=6; untreated p=0.0396; +PIM p=0.0275), or a reduction of dendritic cells (C) ANOVA test, n=6; Untreated p=0.1994; +PIM p=0.3827). (D)-(F) CD3⁻ and CD3⁺ cells dramatically reduce the expression of IFNγ when treated with cell-free supernatant of M. faecis MH23-1 and MH23-2. CD3⁻ cells also showed an increase of IL-22 expression when treated with cell-free supernatant of M. faecis MH23-1 and MH23-2 (ANOVA test, n=7, (D) p<0.0001, (E) p<0.0001, (F) p=0.0189).

FIG. 10 provides a graphical representation showing M. faecis promotes the migration of human gut epithelial cells. (A) Transwell migration assays were employed to study the effect of sterile culture supernatant extracts from M. faecis strains MH23-1 and MH23-2 on the migration of HCT116 colon cancer cells. In serum-starved conditions (0.5% FBS), untreated HCT116 cells and cells treated with Ty medium extract showed comparable background level of migration to the basolateral side of the Transwell chamber. The addition of M. faecis extract to the bottom of the chamber significantly increased the movement of HCT116 cells to the basolateral side compared to the Ty control (Ty, C n=6 technical replicates; MH23-1, MH23-1 n=4 technical replicates, for 3 biological replicates each; Dunnett's multiple comparisons test ** P=0.0035, **** P<0.0001). (B) Representative brightfield images at 10× magnification for Transwell migration experiments. Image size is 703.5 μm×572.5 μm. (C) As a second readout for cell migration, the Incucyte scratch wound assay was performed. The relative wound confluence was measured every two hours after the HCT116 cell monolayer was scratched. 24 hours post scratch, serum-starved HCT116 cells incubated in 0.3× extract from M. faecis strains MH23-1 and MH23-2 showed significantly higher wound confluence, compared to cells treated with Ty medium extract. (Ty, C n=27 technical replicates; MH23-1, MH23-1 n=18 technical replicates, for 3 biological replicates each; Dunnett's multiple comparisons test * p=0.0142, ** p<0.0088). (D) Representative images (10× magnification) of scratch wound migration experiments captured at starting point (0 hours), and 24 as well as 48 hours after the cell monolayer was scratched. Scale bars: 400 μm.

FIG. 11 provides graphical representations of the anti-STAT3 activation activity of M. lactaris. (A) M. lactaris ATCC 29176 cell free culture supernatant and the <3 kDa fraction suppresses STAT3 activation. (B) M. lactaris MH54 cell free culture supernatant and the <3 kDa fraction suppresses STAT3 activation. Samples were compared using an unpaired t-test. p<<0.05, p<0.01, p<0.0001.

FIG. 12 provides graphical representations of the effect of M faecis on barrier integrity. (A) M. faecis MH23-1 and MH23-3 cell free culture supernatant ameloriate IFN-γ induced changes to barrier integrity following 24 hours of treatment relative to the TY medium control as assessed by TEER. (B) M. faecis MH23-1 and MH23-3 cell free culture supernatant ameloriate IFN-γ induced changes to barrier integrity in TEER following 144 hours of treatment relative to the TY medium control as assessed by TEER. (C) M. faecis MH23-3 culture supernatant extract promotes restoration of barrier integrity following IFN-γ treatment relative to the YG/V medium control. Samples were compared using an unpaired t-test. *, p<0.05; **, p<0.01; *** p<0.001: **** p<0.0001.

### BRIEF DESCRIPTION OF THE SEQUENCES

| SEQ ID | |
|---|---|
| NO: | Sequence Description |
| 1 | 16S rRNA sequence of M. faecis strain MH23-1 copy 1 |
| 2 | 16S rRNA sequence of M. faecis strain MH23-1 copy 2 |
| 3 | 16S rRNA sequence of M. faecis strain MH23-1 copy 3 |
| 4 | 16S rRNA sequence of M. faecis strain MH23-1 copy 4 |
| 5 | 16S rRNA sequence of M. faecis strain MH23-1 copy 5 |
| 6 | 16S rRNA sequence of M. faecis strain MH23-1 copy 6 |
| 7 | 16S rRNA sequence of M. faecis strain MH23-2 copy 1 |
| 8 | 16S rRNA sequence of M. faecis strain MH23-2 copy 2 |
| 9 | 16S rRNA sequence of M. faecis strain MH23-2 copy 3 |
| 10 | 16S rRNA sequence of M. faecis strain MH23-2 copy 4 |
| 11 | 16S rRNA sequence of M. faecis strain MH23-2 copy 5 |
| 12 | 16S rRNA sequence of M. faecis strain MH23-2 copy 6 |
| 13 | 16S rRNA sequence of M. faecis strain MH23-3 copy 1 |
| 14 | 16S rRNA sequence of M. faecis strain MH23-3 copy 2 |
| 15 | 16S rRNA sequence of M. faecis strain MH23-3 copy 3 |
| 16 | 16S rRNA sequence of M. faecis strain MH23-3 copy 4 |
| 17 | 16S rRNA sequence of M. faecis strain MH23-3 copy 5 |
| 18 | 16S rRNA sequence of M. faecis strain MH23-3 copy 6 |
| 19 | 16S rRNA sequence of M. faecis strain MH23-4 copy 1 |
| 20 | 16S rRNA sequence of M. faecis strain MH23-4 copy 2 |
| 21 | 16S rRNA sequence of M. faecis strain MH23-4 copy 3 |
| 22 | 16S rRNA sequence of M. faecis strain MH23-4 copy 4 |
| 23 | 16S rRNA sequence of M. faecis strain MH23-4 copy 5 |
| 24 | 16S rRNA sequence of M. faecis strain MH23-4 copy 6 |
| 25 | Partial genome sequence of M. faecis strain MH23-1 |
| 26 | Partial genome sequence of M. faecis strain MH23-2 |
| 27 | Partial genome sequence of M. faecis strain MH23-3 |
| 28 | Partial genome sequence of M. faecis strain MH23-4 |
| 29 | Partial 16S rRNA sequence of M. lactaris strain MH54 1 |
| 30 | Partial 16S rRNA sequence of M. lactaris strain MH54 2 |
| 31 | Partial 16S rRNA sequence of M. lactaris strain MH54 3 |
| 32 | Partial 16S rRNA sequence of M. lactaris strain MH54 4 |
| 33 | Partial genome sequence of M. lactaris strain MH54 |
| 34 | Partial genome sequence of M. lactaris strain MH54 |
| 35 | Partial genome sequence of M. lactaris strain MH54 |
| 36 | Partial genome sequence of M. lactaris strain MH54 |
| 37 | Partial genome sequence of M. lactaris strain MH54 |
| 38 | Partial genome sequence of M. lactaris strain MH54 |

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The term “about” as used herein refers to the usual error range for the respective value readily known to the skilled person in this technical field. Reference to “about” a value or parameter herein includes (and describes) embodiments that are directed to that value or parameter per se.

As used herein, the term “administering,” refers to the placement of an agent (e.g., bacteria) as disclosed herein into a subject by a method or route which results in at least partial delivery of the agent at the desired site. Compositions comprising the compounds disclosed herein can be administered by any appropriate route which results in an effective biological activity or therapeutic effect in the subject. In some embodiments, administration comprises physical human activity (e.g., an injection, act of ingestion, an act of application, and/or manipulation of a delivery device or machine). Such activity can be performed (e.g., by a medical professional and/or the subject being treated).

Specifically, as used herein “administer” and “administration” encompasses embodiments in which one person directs another to consume live bacteria, dead bacteria, spent mediums derived from a bacteria, cell pellets of a bacteria, purified matabolites produced by a bacteria, purified proteins produced by a bacteria, prebiotics, small molecules, or combinations thereof in a certain manner and/or for a certain purpose independently of or in variance to any instructions received from a second person. Non-limiting examples of embodiments include the situation in which one person directs another to consume live bacteria, dead bacteria, spent mediums derived from a bacteria, cell pellets of a bacteria, purified matabolites produced by a bacteria, purified proteins produced by a bacteria, prebiotics, small molecules, or combinations thereof in a certain manner and/or for a certain purpose include when a physician prescribes a course of conduct and/or treatment to a patient, when a parent commands a minor user (such as a child) to consume such a product, when a trainer advises a user (such as an athlete) to follow a particular course of conduct and/or treatment, or when a manufacturer, distributer, or marketer recommends conditions of use to an end user, for example through advertisements or labeling on packing or on other materials provided in association with the sale or marketing of a product. In some embodiments, the disclosed compositions can be administered orally, intravenously, intramuscularly, intrathecally, subcutaneously, sublingually, buccally, rectally, vainally, by the ocular route, by the optic route, nasally, via inhalation, by nebulization, cutaneously, transdermally, or combinations thereof, and formulated for delivery with a pharmaceutically acceptable excipient, carrier or diluent. Of note, although the disclosed compositions encompass multiple formulations and modes of delivery for treatments to ameliorate dysbiosis and its sequelae, it should be noted that live biotherapeutic products such as probiotics are not typically administered intravenously, intramuscularly, or intraperitoneally. These modes of delivery would likely be reserved for small-molecule products of bacterial metabolism.

The terms “administration concurrently” or “administering concurrently” or “co-administering” and the like refer to the administration of a single composition containing two or more actives, or the administration of each active as separate compositions and/or delivered by separate routes either contemporaneously or simultaneously or sequentially within a short enough period of time that the effective result is equivalent to that obtained when all such actives are administered as a single composition. By “simultaneously” is meant that the active agents are administered at substantially the same time, and desirably together in the same formulation. By “contemporaneously” it is meant that the active agents are administered closely in time, e.g., one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example at an interval of about a day to several weeks or months. The active agents may be administered in either order. The term “sequentially” as used herein means that the agents are administered in sequence, for example at an interval or intervals of minutes, hours, days or weeks. If appropriate the active agents may be administered in a regular repeating cycle.

The term “agent” includes a compound that induces a desired pharmacological and/or physiological effect. The term also encompass pharmaceutically acceptable and pharmacologically active ingredients of those compounds specifically mentioned herein including but not limited to salts, esters, amides, prodrugs, active metabolites, analogs and the like. When the above term is used, then it is to be understood that this includes the active agent per se as well as pharmaceutically acceptable, pharmacologically active salts, esters, amides, prodrugs, metabolites, analogs, etc. The term “agent” is not to be construed narrowly but extends to small molecules, proteinaceous molecules such as peptides, polypeptides and proteins as well as compositions comprising them and genetic molecules such as RNA, DNA and mimetics and chemical analogs thereof as well as cellular agents. The term “agent” includes a cell that is capable of producing and secreting a polypeptide referred to herein as well as a polynucleotide comprising a nucleotide sequence that encodes that polypeptide. Thus, the term “agent” extends to nucleic acid constructs including vectors such as viral or non-viral vectors, expression vectors and plasmids for expression in and secretion in a range of cells.

The “amount” or “level” of a biomarker is a detectable level in a sample. These can be measured by methods known to one skilled in the art and also disclosed herein. The expression level or amount of biomarker assessed can be used to determine the response to treatment.

As used herein, “and/or” refers to and encompasses any and all possible combinations of one or more of the associated listed items, as well as the lack of combinations when interpreted in the alternative (or).

The term “anaerobic” means not requiring oxygen for growth. Anaerobic bacterial strains comprise bacterial strains that are obligate anaerobes (i.e., those that are harmed by the presence of oxygen); aerotolerant anaerobes, (i.e., those that cannot use oxygen for growth, but tolerate its presence); and facultative anaerobes (i.e., those that can grow without oxygen, but will use oxygen if it is present).

“Anaerobic conditions” are defined as conditions under which the oxygen concentration in the fermentation medium is too low for the microorganism to use as a terminal electron acceptor. “Anaerobic conditions” can be further defined as conditions under which no or small amounts of oxygen are added to the medium at rates of <3 mmol/L/h, preferably <2.5 mmol/L/h, more preferably <2 mmol/L/h, and most preferably <1.5 mmol/L/h. “Anaerobic conditions” means in particular completely oxygen-free (=0 mmol/L/h oxygen) or with small amounts of oxygen added to the medium at rates of e.g., <0.5 to <1 mmol/L/h. “Anaerobic metabolism” refers to a biochemical process in which oxygen is not the final acceptor of electrons contained in NADH. Anaerobic metabolism can be divided into anaerobic respiration, in which compounds other than oxygen serve as the terminal electron acceptor, and substrate level phosphorylation, in which the electrons from NADH are utilized to generate a reduced product via a fermentative pathway.

The term “carbon source” generally refers to a substrate or compound suitable for sustaining microorganism growth. Carbon sources may be in various forms, including, but not limited to polymers, carbohydrates, alcohols, acids, aldehydes, ketones, amino acids, peptides, etc. For example, these may include monosaccharides (such as glucose, fructose, xylose), oligosaccharides (i.e., sucrose, lactose), polysaccharides (i.e., starch, cellulose, hemicellulose), lignocellulosic materials, fatty acids (i.e., succinate, lactate, acetate), glycerol, etc. or a mixture thereof. The carbon source may be a product of photosynthesis, such as glucose or cellulose.

Monosaccharides used as carbon sources may be the product of hydrolysis of polysaccharides, such as acid or enzymatic hydrolysates of cellulose, starch and pectin. The term “energy source” may be used here interchangeably with carbon source since in chemoorganotrophic metabolism the carbon source is used both as an electron donor during catabolism and as a carbon source during cell growth.

The term “cocci” means having a cellular shape that approximates to spherical, ovoid, or substantially round shape (e.g., when examined under a light microscope). This shape is similar to that of bacterial strains of the genus Staphylococci or Streptococci (e.g., when examined under a light microscope). The characteristic shape of a bacterial strain (such as “cocci”) is a commonly used classification criterion in the field of microbiology.”

Throughout this specification, unless the context requires otherwise, the words “comprise”, “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein, “culturing”, “culture” and the like refer to the set of procedures used in vitro where a population of cells (or a single cell) is incubated under conditions which have been shown to support the growth or maintenance of the cells in vitro. The art recognizes a wide number of formats, media, temperature ranges, gas concentrations etc. which need to be defined in a culture system. The parameters will vary based on the format selected and the specific needs of the individual who practices the methods herein disclosed. However, it is recognized that the determination of culture parameters is routine in nature.

The terms “decrease”, “reduced”, “reduction”, “inhibit”, “suppress”, “attenuate” and the like are all used herein to mean a decrease by a statistically significant amount. In some embodiments, these terms typically mean a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein “reduction”, “suppression”, and “inhibition” does not necessitate a complete inhibition or reduction as compared to a reference level. “Complete inhibition” and the like is a 100% inhibition as compared to a reference level. A decrease can be preferably down to a level accepted as within the range of normal (e.g., for an individual without a given disorder).

The terms “increased”, “increase”, Ienhance”, or “activate” are all used herein to mean an increase by a statistically significant amount. In some embodiments, the terms “increased”, “increase”, “enhance”, or “activate” can mean an increase of at least 10% as compared to a reference level (e.g., the absence of a given treatment or agent) and can include, for example, of at least about 10% as compared to a reference level, for example an increase of at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or up to and including a 100% increase or any increase between 10-100% as compared to a reference level or at least about a 2-fold, or at least about a 3-fold, or at least about a 3-fold, or at least about a 4-fold, or at least about a 5-fold, or at least about a 10-fold increase, or any increase between 2-fold and 10-fold or greater as compared to a reference level. In the context of a marker or symptom, an “increase” is a statistically significant increase in such level.

As used herein, the term “isolated” encompasses a bacterium or other entity or substance that has been (1) separated from at least some of the components with which it was associated when initially produced (whether in nature, such as human stool, or in an experimental setting, such as a Petri plate consisting of artificial growth medium), and/or (2) produced, prepared, purified, and/or manufactured by the hand of man. Isolated bacterial, proteins, metabolites, or combinations thereof may be separated from at least about 10%, about 20%, about 30%, about 40%, about 50%, about 60%, about 70%, about 80%, about 90%, or more of the other components with which they were initially associated. In some embodiments, isolated bacteria, proteins, metabolites, or combinations thereof are more than about 80%, about 85%, about 90%, about 91%, about 92%, about 93%, about 94%, about 95%, about 96%, about 97%, about 98%, about 99% or more than about 99% pure. As used herein, a substance is “pure” if it is substantially free of other components (such as other bacterial species). The terms “purify”, “purifying”, and “purified” refer to a bacterium or other material that has been separated from at least some of the components with which it was associated either when initially produced or generated (e.g., when in nature or in an experimental setting), or during any time after its initial production, as recognized by those skilled in the art of bacterial cultivation or of relevant skill (e.g., chemistry). A bacterium or bacterial population can be considered purified if it is isolated at or after production, such as from a material or environment containing the bacterial or bacterial population, and a purified bacterium or bacterial population can contain other material up to about 10%, about 20%, about 30%, about 40%, about 50%, about 60%, about 70%, about 80%, about 90% or above about 90% and still be considered “isolated”. In some embodiments, purified bacterial and bacterial populations are more than about 80%, about 85%, about 90%, about 91%, about 92%, about 93%, about 94%, about 95%, about 96%, about 97%, about 98%, about 99% or more than about 99% pure. In the instance of bacterial compositions provided herein, the one or more bacterial types present in the composition can be independently purified from one or more other bacteria produced and/or present in the material or environment containing the bacterial type. In some embodiments, a bacterium or population of bacteria is “isolated” if it comprises a single stain of bacteria. In some embodiments, such isolated bacteria can be admixed or administered with other isolated bacteria (e.g., in a defined comsortium of isolated bacteria). Bacterial compositions and the bacterial components thereof are generally purified from residual habitat products.

The term “genome” as used herein includes the DNA comprising the genes (the coding nucleic acid sequences) and the noncoding nucleic acid sequences of a microorganism, and therefore includes introduction of the nucleic acid into, for example, the coding and noncoding DNA of the microorganism.

The term “Gram-variable” means giving a positive result and/or negative result in the Gram strain test (i.e., retaining the colour of the crystal violet staining reagent). Retention of crystal violet staining by a bacterium is linked to the thickness of the peptidoglycan layer in the bacterial cell wall. Gram-positive bacteria have a thicker peptidoglycan layer. Gram-staining is commonly used to help classify bacterial strains in the field of microbiology.

As used herein, the term “gut” is understood to refer to the human gastrointestinal tract, also known as the alimentary canal. The gut includes the mouth, pharynx, oesophagus, stomach, small intestine (duodenum, jejenum, ileum), large intestines (cecum and colon) and rectum. While the entire alimentary canal can be colonized by varying species of microbes, the majority of the gut microbiome, in terms of both numbers of species and biomass, resides in the intestines (small and large).

The terms “marker”, “biomarker” and the like, refer to any compound that can be measured as an indicator of the physiological status of a biological system. The marker may be a biomarker that comprises an amino acid sequence, a nucleic acid sequence and fragments thereof. Exemplary biomarkers include, but are not limited to cytokines, chemokines, growth and angiogenic factors, metastasis related molecules, cancer antigens, apoptosis related proteins, enzymes, proteases, adhesion molecules, cell signalling molecules and hormones. The marker may also be a sugar that, in some embodiments, may not be significantly metabolized in the biological system. The sugar may be, for example, mannitol, lactulose, sucrose, sucralose and combinations of any of the forgoing.

“Measuring” or “measurement” means assessing the presence, absence, quantity or amount (which can be an effective amount) of a given substance within a sample, including the derivation of qualitative or quantitative concentration levels of such substances, or otherwise evaluating the values or categorization of a subject's clinical parameters. Alternatively, the term “assaying,” “detecting” or “detection” may be used to refer to all measuring or measurement as described in this specification.

The term “mucosal healing” as used herein, means an improvement in one or more characteristics of that indicate an impaired mucosal layer. Such characteristics are usually determined by colonic endoscopy and include, but are not limited to, erythema, loss of vascular pattern, friability, bleeding, erosions and ulcers. In some circumstances, mucosal healing refers to a complete amelioration of detrimental effects that characterize an impaired mucosal layer. Alternatively, mucosal healing may refer to a reduction or improvement of one or more of the negative effects that characterize an impaired mucosal layer.

As used herein, the term “pharmaceutical composition” refers to the active agent in combination with a pharmaceutically acceptable carrier (e.g., a carrier commonly used in the pharmaceutical industry). The phrase “pharmaceutically acceptable” is employed herein to refer to those compounds, materials, compositions, and/or dosage forms which are, within the scope of sound medical judgement, suitable for use in contact with the tissues of human beings and animals without excessive toxicitiy, irritation, allergic response, or other problem or complication, commensurate with a reasonable benefit/risk ratio. In some embodiments of any of the aspects, a pharmaceutically acceptable carrier can be a carrier other than water. In some embodiments, any of the aspects a pharmaceutically acceptable carrier can be a cream, emulsion, gel, liposome, nanoparticle, and/or ointment. In some embodiments of any of the aspects, a pharmaceutically acceptable carrier can be an artificial or engineered carrier (e.g., a carrier that the active ingredient would not be found to occur in or within nature).

The term “phylogenetic tree” refers to a graphical representation of the evolutionary relationships of one genetic sequence to another that is generated using defined set of phylogenetic reconstruction algorithms (e.g., parsimony, maximum likelihood, or Bayesian). Nodes in the tree represent distinct ancestral sequences and the confidence of any node is provided by a bootstrap or Bayesian posterior probability, which measures branch uncertainty.

In some embodiments, the term “strain”, refers to a terminal leaf in a phylogenetic tree and is defined by a specific genetic sequence The specific genetic sequence may be a concatenated alignment of 120 ubiquitous single-copy proteins (Parks et al. 2018) extracted from a genome assembly using GTDB-tk (Chaumeil et al. 2020) or other tools known in the art.

The term “clade” refers to the set of members of a phylogenetic tree downstream of a stable node (bootstrap value >90%) in a phylogenetic tree. A clade is a group of related organisms representing all of the phylogenetic descendants of a common ancestor. The clade comprises a set of terminal leaves in the phylogenetic tree that is a distinct monophyletic evolutionary unit.

As used herein, “prebiotic” is understood to mean an ingredient that allows specific changes, both in the composition and/or activity in the gastrointestinal microbiota that may (or may not) confer benefits upon the host. Favoured prebiotics will be those which encourage growth of probiotic compositions or their beneficial functions, but not growth of pathogens nor genes associated with pathogenicity (e.g., toxins).

As used herein, “probiotic” is understood to mean “live microorganisms which when administered in adequate amounts confer a health benefit on the host”, as currently defined by the World Health Orgainization.

The term “species” is defined as a collection of closely related organisms with greater than 97% 16S ribosomal RNA (rRNA) sequence homology and greater than 70% genomic hybridization and sufficiently different from all other organisms so as to be recognized as a distinct unit. Species and other phylogenic identifications are according to the classification known to a person skilled in the art of microbiology.

As used herein, a “subject” means a human or animal. Usually the animal is a vertebrate such as a primate, rodent, domestic animal or game animal. Primates include chimpanzees, cynomologus monkeys, spider monkeys, and macaques (e.g., Rhesus). Rodents include mice, rates, woodchucks, ferrets, rabbits, and hamsters. Domestic and game animals include cows, horses, pigs, deer, bison, buffalo, feline species (e.g., domestic cat), canine species (e.g., dog, fox, wolf), avian species (e.g., chicken, emu, ostrich), and fish (e.g., trout, catfish, and salmon). In some embodiments the subject is a mammal (e.g., a primate (e.g., a human)). The terms “individual”, “patient” and “subject” are used interchangeably herein.

Preferably the subject is a mammal. The mammal can be a human, non-human primate, mouse, rat, dog, cat, horse or cow, but is not limited to these examples. Mammals other than humans can be advantageously used as subjects that represent animal models of inflammatory and autoimmune disorders (e.g., models of gut barrier function). A subject can be male or female.

As used herein, the terms “treat”, “treatment”, “treating” and the like, refer to therapeutic treatments, wherein the object is to reverse, alleviate, ameliorate, inhibit, slow down or stop the progression or severity of a condition associated with a disease or disorder (e.g., an inflammatory or autoimmune disorder). The term “treating” includes reducing or alleviating at least one adverse effect or symptom of a condition, disease or disorder associated with an inflammatory or autoimmune disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration, or palliation of the disease state, remission (whether partial or total), and/or decreased mortality, whether detectable or undetectable. The term “treatment” of a disease also includes providing relief from the symptoms or side-effects of the disease (including palliative treatment). A treatment need not cure a disorder (i.e., complete reversal or absence of disease) to be considered effective.

In some embodiments, sequencing comprises 16S rRNA gene sequencing, which can also be referred to as “16S ribosomal RNA sequencing”, 16S rDNA sequencing” or “16s rRNA sequencing”. Sequencing of the 16S rRNA gene can be used for genetic studies as it is highly conserved between different species of bacteria, but it is not present in eukaryotic species. In addition to highly conserved regions, the 16S rRNA gene also comprises nine hypervariable regions (V1-V9) that vary between species. 16S rRNA gene sequencing typically comprises using a plurality of universal primers that bind to conserved regions of the 16S rRNA gene, PCT amplifying the bacterial 16S rRNA gene regions (including hypervariable regions), and sequencing the amplified 16S rRNA genes with a next-generation sequencing technology as described herein (see, also e.g., U.S. Pat. Nos. 5,654,418; 6,344,316; and 8,889,358; and U.S. Patent Publication Nos. US2013/157,265 and US2018/195,111, which are each incorporated by reference in their entireties).

Each embodiment described herein is to be applied mutatis mutandis to each and every embodiment unless specifically stated otherwise.

### 2. Bacterial Strains

The compositions of the invention comprise a bacterial strain of the genus Mediterraneibacter. The examples demonstrate that bacteria of this genus are useful for treating or preventing diseases associated with an impaired gut barrier function. The preferred bacterial strains are of the species M. faecis.

Mediterraneibacter is a genus of bacteria in the class Clostridia. The scientific classification is as follows: bacteria (kingdom); Firmicutes (phylum); Clostridia (class); Oscillospirales (order); Acutalibacteraceae (family); Mediterraneibacter (genus). Bacteria within the Mediterraneibacter genus are Gram-reaction-variable, non-motile bacteria with a coccoid shape, and are obligate anaerobes. These criteria are important because they can inform the phylogenetic classification of bacterial strains. For instance, the bacterial species M. faecis has previously been classified as belonging to the genus Ruminococcus and Faecalicatena, based on these criteria in particular.

M. faecis strains (previously characterized as Ruminococcus faecis) are described in Kim et al., 2011, with the current taxonomic reclassification described by Togo et al., 2018. The type strain M. faecis Eg2 (=JCM 15917) was isolated from human feces (Kim et al., 2011). The GenBank accession number for the 16S rRNA gene sequence of the M. faecis type strain JCM 15917 is NR_116747.

The breath of the Mediterraneibacter genus and M. faecis species are as defined by Genome Taxonomy Database reference tree, a taxonomic classification system as described in Oren et al., 2015 and Whitman et al., 2018.

The M. faecis bacterium deposited under accession number V21/006223 (i.e., M. faecis MH23-1) was tested in the Examples and is one of the preferred strains of the invention. M. faecis strain MH23-1 was deposited with the international depositary authority National Measurement Institute (NMI, 1/153 Bertie Street, Port Melbourne, Victoria, 3207, Australia) by Microba IP Pty Ltd (388 Queen Street, Brisbane, QLD 4000, Australia) on 31 Mar. 2021 as “Mediterraneibacter faecis MH23-1” and was assigned accession number V21/006223.

Exemplary 16S rRNA sequences for the M. faecis MH23-1 strain that was tested are set forth in SEQ ID NOs: 1-6. Bacterial strains of the species M. faecis may comprise a single 16 rRNA sequence within its genome, or more preferably, may comprise two or more 16S rRNA sequences within its genome (e.g., two copies, three copies, four copies, five copies, six copies, seven copies, eight copies, or more than eight copies). In some of the most preferred embodiments, the M. faecis MH23-1 strain has six copies of the 16S rRNA sequence, as identified in SEQ ID NOs:1-6. In some embodiments, a bacterial strain may be identified as being of the M. faecis MH23-1 strain by determining whether the strain comprises a 16S rRNA sequence that corresponds to any one of SEQ ID NOs: 1-6, by any method known in the art. The genome of M. faecis strain MH23-1 comprises a chromosome and plasmid. A chromosome sequence for M. faecis strain MH23-1 is provided in SEQ ID NO: 1. This sequence was generated using the Illumina NovSeq6000 platform.

Bacterial strains closely related to the strains MH23-1 are also shown in the examples to be effective for treating or preventing inflammatory and autoimmune disorders, through their beneficial effects on restoring gut barrier function.

For example, the M. faecis bacterium deposited under accession number V21/006224 (i.e., M. faecis MH23-2) was tested in the Examples and is another one of the preferred strains of the invention. M. faecis strain MH23-2 was deposited with the international depositary authority National Measurement Institute (NMI, 1/153 Bertie Street, Port Melbourne, Victoria, 3207, Australia) by Microba IP Pty Ltd (388 Queen Street, Brisbane, QLD 4000, Australia) on 31 Mar. 2021 March 2021 as “Mediterraneibacter faecis MH23-2” and was assigned accession number V21/006224.

Exemplary 16S rRNA sequences for the M. faecis MH23-2 strain that was tested are set forth in SEQ ID NOs: 7-12. In some of the most preferred embodiments, the M. faecis MH23-2 strain has six copies of the 16S rRNA sequence, as identified in SEQ ID NOs: 7-12. In some embodiments, a bacterial strain may be identified as being of the M. faecis MH23-2 strain by determining whether the strain comprises a 16S rRNA sequence that corresponds to any one of SEQ ID NOs: 7-12, by any method known in the art. The genome of M. faecis strain MH23-2 comprises a chromosome and plasmid. A chromosome sequence for M. faecis strain MH23-2 is provided in SEQ ID NO: 26.

Furthermore, the M. faecis bacterium deposited under accession number V21/006225 (i.e., M. faecis MH23-3) was also tested in the Examples and is another one of the preferred strains of the invention. M. faecis strain MH23-3 was deposited with the international depositary authority National Measurement Institute (NMI, 1/153 Bertie Street, Port Melbourne, Victoria, 3207, Australia) by Microba IP Pty Ltd (388 Queen Street, Brisbane, QLD 4000, Australia) on 31 Mar. 2021 as “Mediterraneibacter faecis MH23-3” and was assigned accession number V21/006225.

Exemplary 16S rRNA sequences for the M. faecis MH23-3 strain that was tested are set forth in SEQ ID NOs: 13-18. In some of the most preferred embodiments, the M. faecis MH23-3 strain has six copies of the 16S rRNA sequence, as identified in SEQ ID NOs: 13-18. In some embodiments, a bacterial strain may be identified as being of the M. faecis MH23-3 strain by determining whether the strain comprises a 16S rRNA sequence that corresponds to any one of SEQ ID NOs: 13-18, by any method known in the art. The genome of M. faecis strain MH23-3 comprises a chromosome and plasmid. A chromosome sequence for M. faecis strain MH23-3 is provided in SEQ ID NO: 27.

Further still, the M. faecis bacterium deposited under accession number V21/006226 (i.e., M. faecis MH23-4) was also tested in the Examples and is yet another one of the preferred strains of the invention. M. faecis strain MH23-4 was deposited with the international depositary authority National Measurement Institute (NMI, 1/153 Bertie Street, Port Melbourne, Victoria, 3207, Australia) by Microba IP Pty Ltd (388 Queen Street, Brisbane, QLD 4000, Australia) on 31 Mar. 2021 as “Mediterraneibacter faecis MH23-4” and was assigned accession number V21/006226.

Exemplary 16S rRNA sequences for the M. faecis MH23-4 strain that was tested are set forth in SEQ ID NOs: 19-24. In some of the most preferred embodiments, the M. faecis MH23-4 strain has six copies of the 16S rRNA sequence, as identified in SEQ ID NOs: 19-24. In some embodiments, a bacterial strain may be identified as being of the M. faecis MH23-4 strain by determining whether the strain comprises a 16S rRNA sequence that corresponds to any one of SEQ ID NOs: 19-24, by any method known in the art. The genome of M. faecis strain MH23-4 comprises a chromosome and plasmid. A chromosome sequence for M. faecis strain MH23-4 is provided in SEQ ID NO: 28.

In certain embodiments, the bacterial strains of the invention have a 16S rRNA sequence that is at least 97.5%, 98%, 98.5%, 99%, 99.5%, 99.6%, 99.7%, 99.8%, or 99.9% identical to the 16S rRNA sequence of a bacterial strain of M. faecis. Preferably, the bacterial strain of the invention has a 16S rRNA sequence that is at least 97.5%, 98%, 98.5%, 99%, 99.5%, 99.6%, 99.7%, 99.8%, or 99.9% identical to any one of SEQ ID NOs: 1-24. In some preferred embodiments, the bacterial strain of the invention has a 16S rRNA sequence represented by one or more of SEQ ID NOs: 1-6. In some other preferred embodiments, the bacterial strain of the invention has a 16S rRNA sequence represented by one or more of SEQ ID NOs: 7-12. In some alternative preferred embodiments, the bacterial strain of the invention has the 16S rRNA sequence represented by one or more of SEQ ID NOs: 13-18. In still some other preferred embodiments, the bacterial strain of the invention has a 16S rRNA sequence represented by one or more of SEQ ID NOs: 19-24.

The genome of the bacterial strain may comprise each of the 16S rRNA sequences set forth in SEQ ID NOs: 1-6. Alternatively, genome of the bacterial strain may comprise each of the 16S rRNA sequences set forth in SEQ ID NOs: 7-12. Alternatively, the genome of the bacterial strain may comprise each of the 16S rRNA sequences set forth in SEQ ID NOs: 13-18. Alternatively, the genome of the bacterial strain may comprise each of the 16S rRNA sequences set forth in SEQ ID NOs: 19-24.

In certain embodiments, the bacterial strain of the invention has a chromosome with sequence identity to any one of SEQ ID Nos: 25-28. In preferred embodiments, the bacterial strain of the invention has a chromosome with at least 90% sequence identity (e.g., at least 92%, 94%, 95%, 96%, 97%, 97.5%, 98%, 98.5%, 99%, 99.5%, or 100% sequence identity) to any one of SEQ ID NOs: 25-28 across at least 60% (e.g., at least 65%, 70%, 75%, 80%, 85%, 95%, 96%, 97%, 98%, 99% or 100%) of SEQ ID NO: 25-28. For example, the bacterial strain of the invention may have a chromosome with at least 90% sequence identity to any one of SEQ ID NOs: 25-28 across 70% of SEQ ID NOs: 25-28, or at least 90% sequence identity to any one of SEQ ID NOs: 25-28 across 80% of SEQ ID NOs: 25-28, or at least 90% sequence identity to any one of SEQ ID NOs: 25-28 across 90% of SEQ ID NOs: 25-28, or at least 90% sequence identity to any one of SEQ ID NOs: 25-28 across 100% of SEQ ID NOs: 25-28, or at least 95% sequence identity to any one of SEQ ID NOs: 25-28 across 70% of SEQ ID NOs: 25-28, or at least 95% sequence identity to any one of SEQ ID NOs: 25-28 across 80% of SEQ ID NOs: 25-28, or at least 95% sequence identity to any one of SEQ ID NOs: 25-28 across 90% of SEQ ID NOs: 25-28, or at least 95% sequence identity to any one of SEQ ID NOs: 25-28 across 100% of SEQ ID NOs: 25-28, or at least 98% sequence identity to any one of SEQ ID NOs: 25-28 across 70% of SEQ ID NOs: 25-28, or at least 98% sequence identity to any one of SEQ ID NOs: 25-28 across 80% of SEQ ID NOs: 25-28, or at least 98% sequence identity to any one of SEQ ID NOs: 25-28 across 90% of SEQ ID NOs: 25-28, or at least 98% sequence identity to any one of SEQ ID NOs: 25-28 across 100% of SEQ ID NOs: 25-28. A particularly preferred strain of the invention is the Mediterraneibacter faecis strain deposited under accession number V21/006223. This is the exemplary M. faecis MH23-1 strain tested in the DSS mouse model presented in the examples and shown to be effective for treating disease. Therefore, the invention provides a cell, such as an isolated cell, of the M. faecis strain deposited under accession number V21/006223, or a derivative thereof. The invention also provides a composition comprising a cell of the M. faecis strain deposited under accession number V21/006223, or a derivative thereof. The invention also provides a biologically pure culture of the M. faecis strain deposited under accession number V21/006223.

In some alternative embodiments, the invention provides a cell, such as an isolated cell, of the M. faecis strain deposited under accession number V21/006224, or a derivative thereof. The invention also provides a composition comprising a cell of the M. faecis strain deposited under accession number V21/006224, or a derivative thereof. The invention also provides a biologically pure culture of the M. faecis strain deposited under accession number V21/006224.

In some alternative embodiments, the invention provides a cell, such as an isolated cell, of the M. faecis strain deposited under accession number V21/006225, or a derivative thereof. The invention also provides a composition comprising a cell of the M. faecis strain deposited under accession number V21/006225, or a derivative thereof. The invention also provides a biologically pure culture of the M. faecis strain deposited under accession number V21/006225.

In some alternative embodiments, the invention provides a cell, such as an isolated cell, of the M. faecis strain deposited under accession number V21/006226, or a derivative thereof. The invention also provides a composition comprising a cell of the M. faecis strain deposited under accession number V21/006226, or a derivative thereof. The invention also provides a biologically pure culture of the M. faecis strain deposited under accession number V21/006226.

A derivative of the strains deposited under any one of the accession numbers V21/006223, V21/006224, V21/006225, and V21/006226 may be a daughter strain (progeny) or a strain cultured (subcloned) from the original. A derivative of a strain of the invention may be modified, for example at the genetic level, without ablating the biological activity. In particular, a derivative strain of the invention is therapeutically active. A derivative strain will have comparable activity to the original V21/006223, V21/006224, V21/006225, or V21/006226 strain from which it is derived. In particular, a derivative strain will elicit comparable effects in at least one disease model (e.g., colitis) as shown in the Examples, which may be identified by using the culturing and administration protocols described in the Examples. A derivative of any one of the V21/006223, V21/006224, V21/006225, and V21/006226 strains will generally be a biotype of the respective V21/006223, V21/006224, V21/006225, or V21/006226 strain.

References to cells of the M. faecis strains deposited under accession number V21/006223, V21/006224, V21/006225, and V21/006226 encompass any cells that have the same safety and therapeutic efficacy characteristics as the strains deposited under accession numbers V21/006223, V21/006224, V21/006225, and V21/006226, and such cells are encompassed by the invention.

In certain embodiments, the bacterial strains of the invention have a 16S rRNA sequence that is at least 97.5%, 98%, 98.5%, 99%, 99.5%, 99.6%, 99.7%, 99.8%, or 99.9% identical to the 16S rRNA sequence of a bacterial strain of M. lactaris. Preferably, the bacterial strain of the invention has a 16S rRNA sequence that is at least 97.5%, 98%, 98.5%, 99%, 99.5%, 99.6%, 99.7%, 99.8%, or 99.9% identical to any one of SEQ ID NOs: 29-32. In some preferred embodiments, the bacterial strain of the invention has a 16S rRNA sequence represented by SEQ ID NOs: 29-32. The genome of the bacterial strain may comprise one or more of the 16S rRNA sequences set forth in any one of SEQ ID NOs: 29-32. In some embodiments, the genome of the bacterial species may comprise at least three copies of a 16S rRNA sequence.

In certain embodiments, the bacterial strain of the invention has a chromosome with sequence identity to at least one of SEQ ID NOs: 33-38. In preferred embodiments, the bacterial strain of the invention has a chromosome with at least 90% sequence identity (e.g., at least 92%, 94%, 95%, 96%, 97%, 97.5%, 98%, 98.5%, 99%, 99.5%, or 100% sequence identity) to one or more of SEQ ID NOs: 33-38 across at least 60% (e.g., at least 65%, 70%, 75%, 80%, 85%, 95%, 96%, 97%, 98%, 99% or 100%) of SEQ ID NOs: 33-38. For example, the bacterial strain of the invention may have a chromosome with at least 90% sequence identity to one or more of SEQ ID NOs: 33-38 across 70% of SEQ ID NOs: 33-38, or at least 90% sequence identity to one or more of SEQ ID NOs: 33-38 across 80% of SEQ ID NOs: 33-38, or at least 90% sequence identity to one or more of SEQ ID NOs: 33-38 across 90% of SEQ ID NOs: 33-38, or at least 90% sequence identity to one or more of SEQ ID NOs: 33-38 across 100% of SEQ ID NOs 33-38, or at least 95% sequence identity to one or more of SEQ ID NOs: 33-38 across 70% of SEQ ID NOs: 33-38, or at least 95% sequence identity to one or more of SEQ ID NOs: 33-38 across 80% of SEQ ID NOs: 33-38, or at least 95% sequence identity to one or more of SEQ ID NOs: 33-38 across 90% of SEQ ID NOs: 33-38, or at least 95% sequence identity to one or more of SEQ ID NOs: 33-38 across 100% of SEQ ID NOs: 33-38, or at least 98% sequence identity to one or more of SEQ ID NOs: 33-38 across 70% of SEQ ID NOs: 33-38, or at least 98% sequence identity to one or more of SEQ ID NOs: 33-38 across 80% of SEQ ID NOs: 33-38, or at least 98% sequence identity to one or more of SEQ ID NOs: 33-38 across 90% of SEQ ID NOs: 33-38, or at least 98% sequence identity to one or more of SEQ ID NOs: 33-38 across 100% of SEQ ID NOs: 33-38.

### 2.1 Bacteria Biotypes

Bacterial strains that are biotypes of a bacterium deposited under any one of the accession numbers V21/006223, V21/006224, V21/006225, and V21/006226 are also expected to be effective for treating or preventing inflammatory and autoimmune disorders. A biotype is a closely related strain that has the same or very similar physiological and biochemical characteristics.

Strains that are biotypes of a bacterium deposited under any one of the accession numbers V21/006223, V21/006224, V21/006225, and V21/006226, and that are suitable for use in the invention may be identified by sequencing other nucleotide sequences for a bacterium deposited under accession numbers V21/006223, V21/006224, V21/006225, and V21/006226. For example, substantially the whole genome may be sequenced and a biotype strain of the invention may have at least 95%, 96%, 97%, 98%, 99%, 99.5% or 99.9% sequence identity across at least 80% of its whole genome (e.g., across at least 85%, 90%, 95% or 99%, or across its whole genome). Other suitable sequences for use in identifying biotype strains may include hsp60 or repetitive sequences such as BOX, ERIC, (GTG)5, or REP (Masco et al., 2003; Kim et al., 2019). Biotype strains may have sequences with at least 95%, 96%, 97%, 98%, 99%, 99.5% or 99.9% sequence identity to the corresponding sequence of a bacterium deposited under any one of accession numbers V21/006223, V21/006224, V21/006225, and V21/006226.

Alternatively, strains that are biotypes of a bacterium deposited under any one of the accession numbers V21/006223, V21/006224, V21/006225, and V21/006226, and restriction fragment analysis and/or PCR analysis, for example by using fluorescent amplified fragment length polymorphism (FAFLP) and repetitive DNA element (rep)-PCR fingerprinting, or protein profiling, or partial 16S or 23s rRNA sequencing. In preferred embodiments, such techniques may be used to identify other suitable M. faecis strains.

In certain embodiments, strains that are biotypes of a bacterium deposited under any one of accession numbers V21/006223, V21/006224, V21/006225, and V21/006226, and that are suitable for use in the invention are strains that provide the same pattern as a bacterium deposited under any one of accession numbers V21/006223, V21/006224, V21/006225, and V21/006226 when analysed by amplified ribosomal DNA restriction analysis (ARDRA), for example when using Sau3AI restriction enzyme (for exemplary methods and guidance see, for example, Sr\dot{u}tková et al., 2011). Alternatively, biotype strains are identified as strains that have the same carbohydrate fermentation patterns as a bacterium deposited under any one of accession numbers V21/006223, V21/006224, V21/006225, and V21/006226.

In some embodiments, bacterial strains useful in the invention may be identified by routinely profiling the production and consumption of metabolites by a bacterial strain. It is predicted that the bacterial strains described above and elsewhere herein effect production of propionate, lactate, acetate, and formate. Therefore, in some embodiments, the bacterial strains of the invention induce the production in vivo of one or more of the metabolites propionate, lactate, acetate, and formate. Additionally, in some embodiments the bacterial strains of the invention do not produce butyrate.

Other Mediterraneibacter strains that are useful in the compositions and methods of the invention, such as biotypes of a bacterium deposited under any one of accession numbers V21/006223, V21/006224, V21/006225, and V21/006226, may be identified using any appropriate method or strategy, including the assays described in the examples. For instance, strains for use in the invention may be identified by culturing in anaerobic TY or PYG media and/or administering the bacteria to the DSS-induced gut barrier function model and then assessing cytokine/chemokine levels, as described in the Examples. In particular, bacterial strains that have similar growth patterns, metabolic type and/or surface antigens to a bacterium deposited under any one of accession numbers V21/006223, V21/006224, V21/006225, and V21/006226 may be useful in the invention. A useful strain will have comparable immunomodulatory activity to any one of the V21/006223, V21/006224, V21/006225, and V21/006226 strains. In particular, a biotype strain will elicit comparable effects on host gut function. Furthermore, it is expected that a biotype will have a similar effect in a disease model (e.g., colitis, asthma, arthritis, multiple sclerosis and uveitis disease models) and comparable effects on cytokine/chemokine levels to the effects shown in the Examples, and which may be identified by using the culturing and administration protocols described in the Examples.

### 2.2 Bacterial Strain Viability.

In preferred embodiments, the bacterial strains in the compositions of the invention are viable. In preferred embodiments, the bacterial strains in the compositions of the invention are viable and capable of partially or totally colonising the intestine. In some preferred embodiments, the bacterial strains in the compositions of the invention are live. By way of an example, the bacterial strains in the compositions of the invention have not been heat-killed. The bacteria of the invention may have immune modulatory effects that would not be exhibited by non-viable bacteria, for example because non-viable bacteria cannot produce metabolites and interact with the immune system in a different manner. The cell surface of a viable bacterium is also likely to be significantly different to a killed bacterium, in particular a heat-killed bacterium.

In some alternative embodiments, that bacteria are not viable. For example, in some embodiments that bacter are heat-killed.

In some preferred embodiments, the bacterial strain for use in the invention is naturally-occurring. For example, the bacterial strain has been isolated from the mammalian digestive tract.

In some preferred embodiments, the bacterial strain for use in the invention has not been not genetically engineered. For example, the bacterial strain has not been transformed with recombinant DNA.

### 2.3 Antibiotic Resistance

In some embodiments, the bacterial strain for use in the invention is resistant to one of more of tetracycline, bacitracin, amoxicillin, ampicillin, arbekacin and dibekacin, azlocillin, bacampicillin, carbenicillin, ceftobiprole, clarithromycin, doripenem, erythromycin, fusidic acid, gentamicin, grepafloxacin, imipenem, josamycin, meropenem, meziocillin, piperacillin, rifampin, rifaximin, rokitamycin, rosaramicin, roxithromycin, spiramycin, streptomycin, sulfamethoxazole/trimethoprim, telithromycin, ticarcillin, ticarcillin/clavulanate, tosufloxacin, trimethoprim and virginiamycin. In certain embodiments, the bacterial strain for use in the invention is susceptible to Quinopristin-dalfopristin. In some preferred embodiments, the bacterial strain for use in the invention is resistant to tetracycline and/or bacitracin.

In certain embodiments, the bacterial strain for use in the invention is resistant to R-lactam antibiotics. In certain embodiments, the bacterial strain for use in the invention is resistant to tetraycline.

### 3. Compositions

Provided herein are compositions that comprise, consist, or consist essentially of a therapeutically effective amount of a bacterial strain or strains described above and/or elsewhere herein. In some embodiments, the bacteria in the compositions may be identified by strain, species, operational taxonomic unit (OTU), whole genome sequence, 16S rRNA sequence, or other methods known in the art for defining different types of bacteria.

### 3.1 Most Recent Common Ancestor (MRCA)

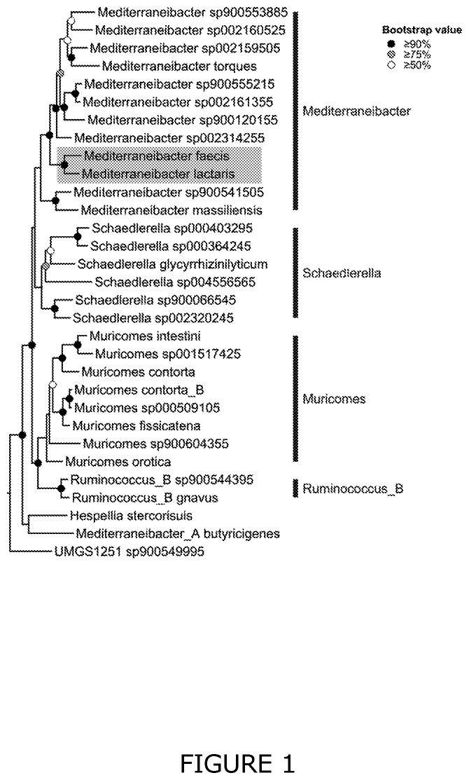

In some embodiments, the compositions comprise an effective amount of a bacterial strain that is a phylogenetic descendant of the MRCA of M. faecis and M. lactaris (FIG. 1). Preferably, the phylogenetic classification is as defined by the GTDB (Parks et al., 2018). In some embodiments, the phylogenetic classification is as defined in release 95 (r95) of the GTDB.

In some embodiments, determining if a bacterial strain is a descendant of a MRCA of M. faecis and M. lactaris may be performed using phylogenetic grouping procedures known in the art. In some embodiments, a rooted phylogenetic tree with M. faecis, M. lactaris and a third taxon of interest (e.g., a taxon to be classified) may be used, with the following the analysis packages being applied: Analyses of Phylogenetics and Evolution (“ape”; https://cran.r-project.org/web/packages/ape/index.html) and Phylogenetic Tool for Comparative Biology (“phytools”; http://cran.r-project.org/web/packages/phytools/index.html), in order to determine whether the taxon of interest is useful for the compositions of the present invention. Both ape and phytools are packages written in the R language for use in studying molecular evolution and phylogenetics. The ape and phytools packages provide methods for phylogenetic and evolutionary analysis and their use is known to one of skill in the art.

In some embodiments, the following script may be used:

library(“ape”)
library(“phytools”)
input.tree = read.tree(file=”tree_file”)
medi = (‘s_Mediterraneibacter_faecis’, ‘s_Mediterraneibacter_lactaris’))
medi.node = getMRCA(input.tree, medi)
medi.tree = extract.clade(input.tree, medi.node)
print(medi.tree$tip.label)

In some embodiments, after the script is run, if the taxon of interest is in the printed list, it is a descendant of a MRCA of the two species.

In other embodiments, different phylogenetic grouping methods known in the art may be used to determine if a bacterial strain is a descendant of a MRCA of M. faecis and M. lactaris (FIG. 1), including methods that use different analysis packages and are based on different programming languages.

In other embodiments, a bacterial species is a member of the family Ruminococcaceae if the species has a 16S rDNA sequence with sequence identity to 16S rDNA sequences from species already identified as a member of the family Ruminococcaceae. In an embodiment, identification of whether a bacterial species is a member of the family Ruminococcaceae is performed using the methods described in Yarza et al., 2014, Nature Reviews Microbiology 12:635-645, and Stackebrandt, E. & Ebers, J., 2006, Microbiol. Today 8:6-9, which are hereby incorporated by reference herein.

3.2 165 rRNA Sequence Identity.

In some embodiments, the 16S rRNA sequence is obtained or determined for a bacterial species to be classified. This query 16S rRNA sequence is compared to 16S rRNA sequences from bacterial species already classified as members of the Mediterraneibacter genus. In some embodiments, the query 16S rRNA sequence is compared to the 16S rRNA sequences set forth in any one of SEQ ID NOs: 1-24. In some alternative embodiments, the query 16S rRNA sequence is compared to the 16S rRNA sequences set forth in any one of SEQ ID NOs: 29-32. In some embodiments, the query 16S rRNA sequence is compared to all known 16S rRNA sequences for bacterial species already classified as members of the Mediterraneibacter genus. In other embodiments, the query 16S rDNA sequence is compared to a subset of all known 16S rDNA sequences for bacterial species already classified as members of the Mediterraneibacter genus. A percent identity between the query sequence and the compared sequences is determined. If the percent identify of the query sequence is determined to be above a defined threshold, then the bacterial species to be classified is classified as member of the Mediterraneibacter genus.

In some embodiments, the threshold sequence identity is 95%. In some embodiments, the threshold sequence identity is 97.5%. In some embodiments, the threshold sequence identity is 99.0%. In some embodiments, the threshold sequence identity is 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9% 99.0%, 99.1%, 99.2%, 99.3%, 99.4%, 99.5%. 99.6%, 99.7%, 99.8%, 99.9% or 100%.

In some embodiments, the 16S rDNA sequence is obtained or determined for a bacterial species to be classified. This query 16S rDNA sequence is compared to 16S rDNA sequences from bacterial species already classified as members of the family Ruminococcaceae. In some embodiments, the query 16S rDNA sequence is compared to the 16S rDNA sequences listed in Table 11. In some embodiments, the query 16S rDNA sequence is compared to all known 16S rDNA sequences for bacterial species already classified as members of the family Ruminococcaceae. In other embodiments, the query 16S rDNA sequence is compared to a subset of all known 16S rDNA sequences for bacterial species already classified as members of the family Ruminococcaceae. A percent identity between the query sequence and the compared sequences is determined. If the percent identify of the query sequence is determined to be above a defined threshold, then the bacterial species to be classified is classified as member of the family Ruminococcaceae.

In some embodiments, the threshold sequence identity is 95%. In some embodiments, the threshold sequence identity is 98.7%. In some embodiments, the threshold sequence identity is 94.8%. In some embodiments, the threshold sequence identity is 94.5%, 94.6%, 94.7%, 94.8%, 94.9%, 95.0%, 95.1%, 95.2%, 95.3%, 95.4%, 95.5%, 95.6%, 95.7%, 95.8%, 95.9%, 96.0%, 96.1%, 96.2%, 96.3%, 96.4%, 96.5%, 96.6%, 96.7%, 96.8%, 96.9%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9% 99.0%, 99.1%, 99.2%, 99.3%, 99.4%, 99.5%. 99.6%, 99.7%, 99.8%, 99.9% or 100%.

### 4. Functional Features of Bacterial Strains

Gut barrier dysregulation is the prototypical function leading to systemic inflammation. As demonstrated in the examples, the bacterial strains of the invention, and compositions comprising said strains, are effective at enhancing gut barrier function.

All inflammatory or autoimmune disorders mediated by gut barrier dysregulation causing systemic inflammation in the subject are applicable for treatment with the bacterial strains described above and/or elsewhere herein.

### 4.1 Gut Barrier Function

Gut barrier (also known as intestinal barrier) function regulates transport and host defense mechanisms at the mucosal interface with the outside world. Transcellular and paracellular fluxes are tightly controlled by membrane pumps, ion channels and tight junctions, adapting permeability to physiological needs. Disturbance at any level, but particularly bacterial translocation due to increased permeability and breakdown of oral tolerance due to compromised epithelial and T cell interaction, can result in inflammation and tissue damage.

The translocation of foreign (i.e., non-host) substances such as lipopolysaccharide (LPS) and other inflammatory compounds from the luminal side of the intestine into the circulating system is inhibited by the epithelial barrier. One of the functions of this epithelial barrier is performed by the tight junctions. Tight junctions, or zonula occludens, are the closely associated areas of two epithelial cells whose membranes join together forming a virtual impermeable barrier to fluid, thereby separating the vascular system from the lumen of the digestive tract. Thus, a reduction of the tight junction barrier function has been demonstrated to result in an increased translocation of undesirable substances such as LPS from intestinal lumen into the circulating system.

The present invention provides methods of restoring or improving gut barrier function in a subject, the method comprising administering to the subject a composition that comprises a bacterial strain of Mediterraneibacter faecis, to thereby restore or improve the gut barrier function in the subject. As used in this specification, gut barrier integrity refers to a measure of gut barrier function. High gut barrier integrity can be associated with a lack of gut or intestinal permeability, wherein a high level of gut permeability is indicative of low gut barrier integrity. In a related embodiment, the invention also provides methods of maintaining healthy or normal gut barrier function. Such methods may be used to prevent gut barrier dysregulation is subjects considered to be at high risk of gut barrier dysregulation (e.g., subjects in remission of IBD).

In some embodiments, at least one biomarker measured in a sample (and, in particular, a biological sample) is used to assess the change, in particular, an improvement, in the gut barrier integrity of a subject.

In some embodiments of the methods and uses provided in this specification, the composition comprising a bacterial strain of M. faecis may increase or decrease the levels of one or more biomarkers of gut barrier integrity in a sample from a subject. In some embodiments, depending on the particular biomarker, either an increase or a decrease in the level of the marker is indicative of an increase in gut barrier integrity and/or a decrease in gut permeability. In some embodiments, the biomarker is selected from a cytokine, chemokine, growth factor, angiogenic factor, enzyme, protease, adhesion molecule, cell signalling molecule, hormone or sugar. In some embodiments, the biomarker comprises a cytokine. In some embodiments, the marker comprises a chemokine. In some embodiments, the marker comprises a growth factor. In some embodiments, the marker comprises an angiogenic factor. In some embodiments, the marker comprises an enzyme. In some embodiments, the marker comprises a protease. In some embodiments, the marker comprises an adhesion molecule. In some embodiments, the marker comprises a cell signalling molecule. In some embodiments, the marker comprises a hormone. In some embodiments, the marker comprises a sugar.

This specification provides assays for biomarkers of intestinal permeability. Biological samples from the subject such as blood (plasma, or serum) or tissue may be used to measure levels of any suitable biomarker including one or more of LPS, lipopolysaccharide binding protein (LPSBP), intestinal fatty acid binding protein (IFABP), zonulin, bacterial and/or 16S rRNA, but is not limited to these markers. LPS, I-FABP and Zonulin may be measured by enzyme-linked immunosorbent assay (“ELISA”). Techniques and kits for ELISA are well known to those in the art. In some embodiments, elevated LPS, I-FABP and/or Zonulin, when compared to a control in blood, serum, saliva, urine and/or plasma, is used as an indicator of increased intestinal permeability, and, thus, lower gut barrier integrity.

LPSBP may also be measured by ELISA. In some embodiments, significant changes in LPSBP either higher or lower, when compared to a control, may be used as an indicator of increased intestinal permeability and can confirm a reduced gut barrier integrity.

In some embodiments, increases in bacterial 16S rRNA is used as an indicator of increased intestinal permeability, and, therefore, a reduction in gut barrier integrity. Bacterial 16S rRNA may be purified from blood, serum, organ tissue or urine using standard nucleic acid isolation protocols. These are, for example, commercially available. The isolated nucleic acids may be detected by qPCR amplification using primers specific for bacterial 16S rRNA sequences or amplification using primers specific for bacterial 16S rRNA and sequencing the resultant amplicons.

Tight junction proteins that are expressed by the intestinal epithelial cells and regulate intestinal permeability may also be used as biomarkers of intestinal permeability. In some embodiments, tight junction proteins are assayed to determine alterations in intestinal permeability and gut barrier integrity. In some embodiments, the proteins measured may include, but are not limited to, claudins, occludin, ZO-1, and E-cadherin (adherens junction) proteins. Other tight junction proteins may also be assayed. In some embodiments, the tight junction proteins are measured using an immunohistochemical stain. In some embodiments, the tight junction proteins are measured using ELISA.

In some embodiments, plasma citrulline is assayed to determine alterations in intestinal permeability and gut barrier integrity. A reduction in plasma citrulline levels corresponds to a loss in epithelial cell mass indicating an increase in gut barrier permeability.

In some embodiments, the method includes oral administration of an insoluble sugar such as sucralose, collection of a bodily fluid such as urine or blood after one or more defined periods of time, and measurement of the insoluble sugar contained in the bodily fluid through standard clinical analytical techniques. The insoluble sugars may include, but are not limited to, mannitol, lactulose, sucrose, sucralose and combinations of any of the foregoing.

In some embodiments, gut barrier integrity is measured using an in vitro assay. A particularly preferred in vitro assay suitable for measuring gut barrier function is by trans-epithelial electrical resistance (TEER). Such assays are well known in the field (e.g., Srinivasan, 2015; and Lea, 2015).

### 4.2 Mucosal Healing

Mucosal healing has become an important endpoint to assess the therapeutic effect in inflammatory and autoimmune disorders. The definition of full mucosal healing currently used in IBD (e.g., CD and UC) clinical trials is the “complete absence of all inflammatory and ulcerative lesions”, but this definition lacks validation and does not include mucosal improvement and grading of mucosal healing.

Mucosal healing is predominantly defined by endoscopic assessment of intestinal inflammation. In order to evaluate the presence or absence of mucosal healing on endoscopy, various endoscopic scoring systems have been developed. These indices allow for the determination of improvements of endoscopic lesions, even when the rather rigid endpoint of mucosal healing and thereby the total disappearance of all mucosal ulcerations is not met. The endoscopic component of the clinical Mayo score, introduced in 1987, is currently the most used score of the mucosal layer in clinical practice (see, Schroeder et al., 1987). It includes the variables erythema, loss of vascular pattern, friability, bleeding, erosions and ulcers, and ranges from 0 to 3. MH is classically considered to be a score of 0 (normal mucosa) or 1 (mucosal erythema, decreased vascular pattern, mild friability) (D'Haens, 2007).

In some other embodiments, mucosal healing is determined to have occurred when the patient is determined to have an endoscopy sub-score of 0 or 1 as assessed by flexible sigmoidoscopy. In certain such embodiments, patients who experience mucosal healing are determined to have an endoscopy sub-score of 0.

Both corticosteroids and aminosalicylates have been used for decades and are among the most commonly prescribed drugs for repairing the mucous layer (e.g., in patients with UC) (Carvalho and Cotter, 2017). The mechanisms through which they reduce mucosal inflammation include controlling nuclear factor (NF)-kB expression and inflammatory cytokines (directly modulating cell migration and proliferation of epithelial cell lines. Anti-TNF drugs (e.g., infliximab, adalimumab, and golimumab) act at several steps of mucosal injury, restricting the inflammatory infiltrate and T cell proliferation within the lamina propria (Baert, 1999), and downregulating the expression of metalloproteinases and proinflammatory molecules (Baert, 1999). They also act on the regenerative process, restoring the protective capabilities of the mucosa by reinforcing intestinal permeability and mucosal secretion, activating fibroblasts, and maintaining epithelia regeneration (Suenaert, 2002).

Other measures of assessing mucosal healing are well known in the art, including the measurement of biomarkers C-reactive protein and calprotectin. An advantage of using in vitro biomarker assays for the assessment of mucosal healing is that such assays are typically far less invasive for the subject. Histopathology is another measure of inflammation, which has been cited as being particularly informative for mucosal healing.

### 4.3 STAT3 Signalling Pathway

Cytokine pathways mediate a broad range of biological functions, including many aspects of inflammation and immunity. The Janus kinases (JAK), including JAK1, JAK2, JAK3 and tyrosine kinase 2 (TYK2), are cytoplasmic tyrosine kinases that associate with type I and type II cytokine receptors and regulate cytokine signal transduction. Cytokine engagement with cognate receptors triggers activation of receptor associated JAKs and this leads to JAK-mediated tyrosine phosphorylation of signal transducer and activator of transcription (STAT) proteins and ultimately transcriptional activation of specific gene sets (Schindler et al., 2007, J. Biol. Chem. 282: 20059-63). Cytokine receptors are typically functional as heterodimers, and as a result, more than one type of JAK kinase is usually associated with a cytokine receptor complex. The specific JAKs associated with different cytokine receptor complexes have been determined in many cases through genetic studies and corroborated by other experimental evidence.

STAT3 plays an important role in the activation of several autoimmune and inflammatory disorders, including IBD. The bacterial strains of the present invention significantly suppress IL-23-mediated STAT3 activation. Thus, the present invention provides methods of suppressing or otherwise inhibiting STAT3 signalling in a subject (i.e., IL-23-mediated STAT3 signalling), the method comprising administering to the subject a composition that comprises bacterial strain as described above and/or elsewhere herein. Accordingly, in some embodiments the bacterial strains described herein directly or indirectly suppress STAT3 activity. In some embodiments, the strain of M. faecis produces a bioactive molecule that binds directly to a STAT3 polypeptide. In some alternative embodiments, the bacterial strain is an indirect inhibitor of STAT3 activation, for example, by binding to a molecule upstream of STAT3 in the IL-23-mediated STAT3 signalling pathway, or by binding to a molecule that regulates STAT3 activity (e.g., ubiquitination). By way of an illustrative example, the bioactive agent may directly bind or antagonize any one of IL-23, JAK2, or TYK2 in order to suppress the IL-23-mediated STAT3 signalling pathway.

### 4.4 Th17 Inflammatory Response

Some bacterial compositions of the invention are effective for reducing the Th17 inflammatory response. In particular, treatment with the compositions described above and elsewhere herein may modulate Th17 pathway cytokines (including TNF, IL-22, IL-21, and IL-17), and result in clinical improvements in animal models of conditions mediated by the Th17 pathway. Therefore, the compositions of the invention may be useful for treating or preventing inflammatory and autoimmune disorders, and in some embodiments, diseases or conditions mediated by Th17. In particular, the compositions of the invention may be useful for reducing or preventing elevation of the Th17 inflammatory response.

Th17 cells are a subset of T helper cells that produce, among other cytokines, IL-17A, IL-17F, IL-21 and IL-22. Th17 cell differentiation may be driven by IL-23. These cytokines and others form important parts of the Th17 pathway, which is a well-established inflammatory signalling pathway that contributes to and underlies a number of inflammatory and autoimmune disorders (as described in, for example, Ye, 2015; Fabro, 2015; Yin, 2014; Cheluvappa, 2014; Schieck, 2014; Balato, 2014). Some diseases that are mediated by Th17 can be ameliorated or alleviated by repressing the Th17 pathway, which may be through a reduction in the differentiation of Th17 cells or a reduction in their activity or a reduction in the level of Th17 pathway cytokines. Diseases mediated by the Th17 pathway may be characterised by increased levels of cytokines produced by Th17 cells, such as IL-17A, IL-17F, IL-21, IL-22, IL-26, IL-9 (reviewed in Monteleone, 2011). Diseases mediated by the Th17 pathway may be characterised by increased expression of Th17-related genes, such as STAT3 or IL-23 receptor. Diseases mediated by the Th17 pathway may be associated with increased levels of Th17 cells.

IL-17 is a key cytokine that links T cells activation to neutrophils activation and mobilization, hence IL-17 plays a pivotal role in innate immunity. However, due to its role in neutrophils activation, can contribute to inflammatory autoimmune diseases such as inflammatory bowel disease, psoriasis, and rheumatoid arthritis. IL-17 as used herein may refer to any member of the IL-17 family, including IL-17A, IL-17B, IL-17C, IL-17D, IL-17E, and IL-17F. IL-17-mediated diseases and conditions are characterised by high expression of IL-17 and/or the accumulation, or presence of IL-17-positive cells in a tissue affected by the disease or condition. Similarly, IL-17-mediated diseases and conditions are diseases and conditions that are exacerbated by high IL-17 levels or an increase in IL-17 levels, and that are alleviated by low IL-17 levels or a reduction in IL-17 levels. The IL-17 inflammatory response may be local or systemic.

Examples of diseases and conditions that may be mediated by the Th17 pathway include (but are not limited to) inflammatory bowel disease (such as Crohn's disease and ulcerative colitis); multiple sclerosis; arthritis (such as rheumatoid arthritis, osteoarthritis, psoriatic arthritis, and juvenile idiopathic arthritis); neuromyelitis optica (Devic's disease); ankylosing spondylitis; spondyloarthritis; psoriasis; systemic lupus erythematosus; celiac disease; asthma (such as allergic asthma or neutrophilic asthma); chronic obstructive pulmonary disease (COPD); cancer (such as breast cancer, colon cancer, lung cancer or ovarian cancer); uveitis; scleritis; vasculitis; Behcet's disease; atherosclerosis; atopic dermatitis; emphysema; periodontitis; allergic rhinitis; and allograft rejection. Accordingly, in some aspects the present invention provides methods for treating or preventing one or more of these conditions or diseases, by administering a composition as described above and/or elsewhere herein. In further preferred embodiments, these conditions or diseases are mediated by the STAT3 signalling pathway. In further preferred embodiments, these conditions or diseases are mediated through the Th17 pathway.

In certain embodiments, the present invention provides methods compositions of the invention are for use in a method of reducing Th17 cell differentiation in the treatment or prevention of a disease or condition mediated by the Th17 pathway. In certain embodiments, the compositions of the invention are for use in treating or preventing an inflammatory or autoimmune disorder, wherein said treatment or prevention is achieved by reducing or preventing elevation of the Th17 inflammatory response. In certain embodiments, the compositions of the invention are for use in treating a patient with an inflammatory or autoimmune disorder, wherein the patient has elevated IL-17 levels or elevated Th17 cells or is exhibiting a Th17 inflammatory response. In certain embodiments, the patient may have been diagnosed with a chronic inflammatory or autoimmune disorder or condition, or the composition of the invention may be for use in preventing an inflammatory or autoimmune disorder or condition developing into a chronic inflammatory or autoimmune disorder or condition. In certain embodiments, the disease or condition may not be responsive to treatment with TNF inhibitors. These uses of the invention may be applied to any of the specific disease or conditions listed in the preceding paragraph.

The Th17 pathway are often associated with chronic inflammatory and autoimmune disorders, so the compositions of the invention may be particularly useful for treating or preventing chronic diseases or conditions as listed above. In certain embodiments, the compositions are for use in patients with chronic disease. In certain embodiments, the compositions are for use in preventing the development of chronic disease.

The compositions of the invention may be useful for treating diseases and conditions mediated by the Th17 pathway and for addressing the Th17 inflammatory response, so the compositions of the invention may be particularly useful for treating or preventing chronic disease, treating or preventing disease in patients that have not responded to other therapies (such as treatment with TNF inhibitors), and/or treating or preventing the tissue damage and symptoms associated with Th17 cells. For example, IL-17 is known to activate matrix destruction in cartilage and bone tissue and IL-17 has an inhibitory effect on matrix production in chondrocytes and osteoblasts, so the compositions of the invention may be useful for treating or preventing bone erosion or cartilage damage.

In certain embodiments, treatment with compositions of the invention provides a reduction or prevents an elevation in IL-17 levels, in particular IL-17A levels. In certain embodiments, treatment with compositions of the invention provides a reduction or prevents an elevation in IFN-γ or IL-6 levels. Such reduction or prevention of elevated levels of these cytokines may be useful for treating or preventing inflammatory and autoimmune disorders and conditions, in particular those mediated by the Th17 pathway.

### 4.5 Th1 Inflammatory Response

CD4⁺ T cells play an important role in inflammatory disease/disorder pathogenesis, with many subsets of CD4⁺ T cells having been identified as drivers in perpetuating chronic intestinal inflammation (see, Imam et al., 2018). For example, T helper type 1 (Th1) cells accumulate in the intestinal tract of individuals with IBD, and are directly associated with disease. Interferon-γ (IFN-γ) is the defining cytokine produced by Th1 cells. During intestinal inflammation IFN-γ in combination with TNF is proposed to drive intestinal epithelial cell β-catenin signalling and limit their differentiation and proliferation (Imam et al., 2018).

### 5. Methods of Treatment

In some embodiments, the present invention provides methods of treating or preventing an inflammatory or autoimmune disorder in a subject, the methods comprising administering to the subject a bacterial strain as described above and/or elsewhere herein.

Suitably, the inflammatory or autoimmune disorder is selected from the group comprising: an inflammatory bowel disease (such as Crohn's disease or ulcerative colitis); asthma (such as allergic asthma or neutrophilic asthma); arthritis (such as rheumatoid arthritis, osteoarthritis, psoriatic arthritis, or juvenile idiopathic arthritis); fatty liver disease (such as nonalcoholic fatty liver disease (NAFLD)); ankylosing spondylitis; psoriasis; systemic lupus erythematosus (SLE); scleroderma; Sjogren's syndrome; vasculitis; and type 1 diabetes mellitus.

### 5.1 Inflammatory Bowel Disease (IBD)

The examples demonstrate that the compositions of the invention have a beneficial restorative effect on gut barrier function and that they also have anti-inflammatory properties, and so they may be useful in the treatment of IBD. Accordingly, in some embodiments the invention provides a composition comprising a bacterial strain of the genus Mediterraneibacter for use in a method of treating or preventing an inflammatory bowel disease. The inventors have identified that treatment with Mediterraneibacter strains reduces severity of colitis in a mouse model of disease. Thus, the compositions of the invention may be useful in the treatment of inflammatory diseases. In some embodiments, the compositions of the invention are for use in the treatment or prevention of an IBD. In some embodiments, the invention provides methods of treating or preventing ulcerative colitis. In some embodiments, the invention provides methods of treating or preventing of Crohn's disease. In certain embodiments, the invention provides methods of treating or preventing ulcerations and/or bleeding in the treatment of an IBD, in particular in the treatment of colitis and ulcerative colitis. In preferred embodiments, the invention provides a method of treating or preventing IBD in a subject, the method comprising administering to the subject a composition comprising a bacterial strain of the species Mediterraneibacter faecis. In further preferred embodiments, the invention provides a method of treating or preventing colitis, (particularly ulcerative colitis) in a subject, the method comprising administering to the subject a composition comprising a bacterial strain of the species Mediterraneibacter faecis. In further preferred embodiments, the invention provided methods of reducing at least one side effect of colitis (particularly ulcerative colitis), including ulcerations and/or bleeding.

IBD is a complex disease that can be caused by multiple environmental and genetic factors. Factors contributing to the onset of IBD include diet, microbiota, intestinal permeability, and genetic susceptibility to increased inflammatory response to gut infection. Symptoms of inflammatory bowel disease include abdominal pain, vomiting, diarrhea, rectal bleeding, severe internal cramps/muscle spasms in the pelvic region, weight loss and anaemia. In certain embodiments, the compositions are for use in reducing one or more symptoms associated with IBD. In certain embodiments, the compositions of the invention are for use in preventing one or more symptoms of IBD.

IBD may accompany other diseases or conditions, such as cardiovascular disease, neuropsychological disorders, and metabolic syndrome. In certain embodiments, the compositions of the invention are for use in the treatment or prevention of one or more diseases or conditions that accompany IBD.

IBD is generally diagnosed by biopsy or colonoscopy. Measurements of faecal calprotectin is useful for the preliminary diagnosis of IBD. Other laboratory test for the diagnosis of IBD include, complete blood count, erythrocyte sedimentation rate, comprehensive metabolic panel, faecal occult blood test or C-reactive protein test. Typically, a combination of laboratory testing and biopsy/colonoscopy will be used to confirm diagnosis of IBD. In certain embodiments, the compositions of the invention are for use in a subject diagnosed with IBD.

In certain embodiments the IBD is Crohn's disease and/or ulcerative colitis. As broadly described above, studies have shown that several inflammatory cytokines are upregulated in the inflammatory mucosa of patients with Crohn's disease and ulcerative colitis, including but not limited to STAT3 signalling and NFκB signalling pathway-mediated cytokines (e.g., IL-17, TNF, IL-21, IL-22). Therefore, inhibition of STAT3 signalling pathway-mediated cytokine activity and/or NFκB signalling pathway-mediated cytokines may be useful in the treatment of Crohn's disease and ulcerative colitis. In certain embodiments, the compositions of the invention are for use in the treatment or prevention of Crohn's disease and/or ulcerative colitis.

Crohn's disease and ulcerative colitis are complex diseases with an array of probable causes, including genetic risk factors, diet, other lifestyle factors, such as smoking and alcohol consumption, and microbiome composition. Crohn's disease can manifest anywhere along the GI tract, whereas ulcerative colitis is typically prevalent in the large intestine and colon.

Gastrointestinal symptoms of IBD range from mild to severe and include abdominal pain, diarrhea, faecal blood, ileitis, increased bowel movements, increased flatulence, intestinal stenosis, vomiting, and perianal discomfort. The compositions of the invention may be for use in the treatment of prevention of one or more gastrointestinal symptoms of Crohn's disease and/or ulcerative colitis.

Systemic symptoms of Crohn's disease and ulcerative colitis include growth defects, such as the inability to maintain growth during puberty, decreased appetite, fever and weight loss. Extra-intestinal features of Crohn's disease include uveitis, photobia, episcleritis, gall stones, seronegative spondyloarthropathy, arthritis, enthesitis, erythema nodosum, pyoderma gangrenosum, deep venous thrombosis, pulmonary embolism, autoimmune haemolytic anaemia, clubbing and osteoporosis. Extra-intestinal features are additional conditions associated with Crohn's disease and/or ulcerative colitis that manifest outside the GI tract. Subjects with Crohn's disease also exhibit increased susceptibility to neurological complications such as seizures, strokes, myopathy, peripheral neuropathy, headache and depression. In certain embodiments, the compositions of the invention are for use in the treatment or prevention of one or more systemic symptoms of Crohn's disease and/or ulcerative colitis. In certain embodiments, the compositions of the invention are for use in the treatment or prevention of one or more extra-intestinal features of Crohn's disease and/or ulcerative colitis.

The diagnosis of Crohn's disease and ulcerative colitis usually involves carrying out multiple tests and surgical procedures, such as gastroscopy and/or colonoscopy and biopsy, typically of the ileum, radiologic tests, complete blood counts, C-reactive protein tests and erythrocyte sedimentation rates. In certain embodiments, the compositions of the invention are for use in subjects diagnosed with Crohn's disease or ulcerative colitis. In some embodiments, compositions of the invention are for use in treating a subject who has been diagnosed with Crohn's disease or ulcerative colitis.

Crohn's disease and ulcerative colitis are classified depending on the extent of the region of the GI tract affected (Gasche et al., 2000). A Crohn's disease of both the ileum and colon is classified as Ileocolic Crohn's. In some embodiments, the compositions are for use in the treatment or prevention of Ileocolic Crohn's. In some embodiments, the compositions are for use in a subject diagnosed with Ileocolic Crohn's/Crohn's ileitis is classified if only the ileum is affected. Crohn's colitis is classified if only the colon is affected. In certain embodiments, the compositions are for use in the treatment or prevention of Crohn's ileitis. In some embodiments, the compositions are for use in a subject diagnosed with Crohn's ileitis. In certain embodiments, the compositions are for use in the treatment or prevention of Crohn's colitis. In some embodiments, the compositions are for use in a subject diagnosed with Crohn's colitis.

Crohn's disease and ulcerative colitis may be treated with a number of therapeutic agents, such as corticosteroids, such as prednisone, immunosuppressive agents, such as azathioprine, or biologics, such as infliximab, adalimumab, and golimumab, vedolizumab and etrolizumab. In certain embodiments, the compositions of the invention are for use in the treatment or prevention of Crohn's disease or ulcerative colitis in combination with an additional therapeutic agent, including but not limited to those listed above. In certain embodiments, the additional therapeutic agent is for use in the treatment or prevention of Crohn's disease and/or ulcerative colitis.

### 5.2 Autoimmune Disorders

In humans, signs of intestinal inflammation are detectable before the clinical onset of many autoimmune disorders, such as type 1 diabetes (T1D) (Bosi, 2006). Similarly, augmented gut permeability appears before the development of insulitis in diabetes-prone rats in comparison with diabetes-resistant rats (Meddings, 1999; Neu, 2005). Those findings indicate that the breakage of gut barrier integrity with subsequent increased antigen trafficking and occurrence of low-grade intestinal inflammation precede the onset of T1D and are directly related to its pathogenesis, rather than secondary to diabetes-induced metabolic alterations (i.e., hyperglycemia). The gastrointestinal barrier is a fundamental gatekeeper to avoid the contact between luminal content and the human body. The barrier is composed of a mucus layer and an intestinal epithelial barrier (IEB), and both are crucial to prevent the passage of commensal bacteria, pathogens, and food antigens from the lumen into the gut tissue and systemic circulation. The IEB is a single layer of epithelial cells held together by a complex junctional system composed of tight junctional adhesion molecules (JAMs), tricellulin, and angulins whose interaction between themselves and with intracellular scaffolding proteins, i.e., zonula occludens proteins (ZOs), is fundamental to maintain tight junction integrity and control paracellular trafficking. In patients and rat models of T1D alterations of the IEB have been reported in association with gut inflammation (Meddings, 1999; Sapone, 2006). Furthermore, the importance of the gut mucus layer, an important gut barrier containing immunoregulatory molecules such as antimicrobial peptides and mucins, has recently been reported (see, Sorini et al., 2019).

In some embodiments, bacterial strains from the species Mediterraneibacter faecis may provide therapeutic benefits in the treatment or prevention of asthma, such as allergic asthma or neutrophilic asthma. In certain embodiments, the compositions of the invention are for use in the treatment or prevention of asthma in a subject. In certain embodiments, the invention provides a composition comprising a bacterial strain of the species Mediterraneibacter faecis for use in the treatment or prevention of asthma.

In some embodiments, bacterial strains from the species M. faecis may provide therapeutic benefits in the treatment or prevention of GVHD. In certain embodiments, the compositions of the invention are for use in the treatment or prevention of GVHD in a subject. In preferred embodiments, the invention provides a composition comprising a bacterial strain of the species M. faecis for use in the treatment or prevention of GVHD.

In some embodiments, bacterial strains from the species M. faecis may provide therapeutic benefits in the treatment or prevention of arthritis, such as rheumatoid arthritis, osteoarthritis, psoriatic arthritis, or juvenile idiopathic arthritis. In certain embodiments, the compositions of the invention are for use in the treatment or prevention of arthritis in a subject. In certain embodiments, the invention provides a composition comprising a bacterial strain of the species M. faecis for use in the treatment or prevention of arthritis.

In some embodiments, bacterial strains from the species M. faecis may provide therapeutic benefits in the treatment or prevention of multiple sclerosis. In certain embodiments, the compositions of the invention are for use in the treatment or prevention of multiple sclerosis in a subject. In certain embodiments, the invention provides a composition comprising a bacterial strain of the species M. faecis for use in the treatment or prevention of multiple sclerosis.

In some embodiments, bacterial strains from the species M. faecis may provide therapeutic benefits in the treatment or prevention of psoriasis. In certain embodiments, the compositions of the invention are for use in the treatment or prevention of psoriasis in a subject. In certain embodiments, the invention provides a composition comprising a bacterial strain of the species M. faecis or use in the treatment or prevention of psoriasis.

In some embodiments, bacterial strains from the species M. faecis may provide therapeutic benefits in the treatment or prevention of systemic lupus erythematosus (SLE). In certain embodiments, the compositions of the invention are for use in the treatment or prevention of SLE in a subject. In certain embodiments, the invention provides a composition comprising a bacterial strain of the species M. faecis for use in the treatment or prevention of SLE.

In some embodiments, bacterial strains from the species M. faecis may provide therapeutic benefits in the treatment or prevention of allograft rejection. In certain embodiments, the compositions of the invention are for use in the treatment or prevention of allograft rejection in a subject. In certain embodiments, the invention provides a composition comprising a bacterial strain of the species M. faecis for use in the treatment or prevention of allograft rejection.

### 6. Formulations

In some embodiments, the compositions of the invention comprise fewer than 40 different bacterial strains. In some embodiments, the composition comprises fewer than 30 different bacterial strains. In some embodiments, the composition comprises fewer than 20 different bacterial strains. In some embodiments, the composition comprises fewer than 10 different bacterial strains. In some embodiments, the composition comprises fewer than 5 different bacterial strains. In some preferred embodiments, the composition comprises fewer than three different bacterial strains. In some embodiments, the composition does not comprise bacteria of the genus Clostridium.

The compositions of the invention comprise bacteria (i.e., live bacteria and/or killed bacteria). In preferred embodiments of the invention, the composition is formulated in freeze-dried form. The composition of the invention may comprise granules or gelatin capsules, for example hard gelatin capsules, comprising a bacterial strain of the invention. Preferably, the composition of the invention comprises lyophilised bacteria. Lyophilisation of bacteria is a well-established procedure and relevant guidance is available in, for example, references (Miyamoto-Shinohara, 2008; and Day & Stacey, 2007).

The composition of the invention may comprise a live, active bacterial culture. The examples demonstrate that cultures of the bacteria of the invention are therapeutically effective.

In some embodiments, the bacterial strain in the composition of the invention has not been inactivated, for example, has not been heat-inactivated. In some embodiments, the bacterial strain in the composition of the invention has not been killed, for example, has not been heat-killed. In some embodiments, the bacterial strain in the composition of the invention has not been attenuated, for example, has not been heat-attenuated. For example, in some embodiments, the bacterial strain in the composition of the invention has not been killed, inactivated and/or attenuated. For example, in some embodiments, the bacterial strain in the composition of the invention is live. For example, in some embodiments, the bacterial strain in the composition of the invention is viable. For example, in some embodiments, the bacterial strain in the composition of the invention is capable of partially or totally colonising the intestine. For example, in some embodiments, the bacterial strain in the composition of the invention is viable and capable of partially or totally colonising the intestine.

In some embodiments, the composition comprises a mixture of live bacterial strains and bacterial strains that have been killed. In preferred embodiments, the composition of the invention is encapsulated to enable delivery of the bacterial strain to the intestine. Encapsulation protects the composition from degradation until delivery at the target location through, for example, rupturing with chemical or physical stimuli such as pressure, enzymatic activity, or physical disintegration, which may be triggered by changes in pH. Any appropriate encapsulation method may be used. Exemplary encapsulation techniques include entrapment within a porous matrix, attachment or adsorption on solid carrier surfaces, self-aggregation by flocculation or with cross-linking agents, and mechanical containment behind a microporous membrane or a microcapsule. Guidance on encapsulation that may be useful for preparing compositions of the invention is widely available in the art (for example, in Mitropoulou, 2013; and Kailasapathy, 2002).

The composition may be administered orally and may be in the form of a tablet, capsule or powder. Encapsulated products are preferred because bacteria of the genus Mediterraneibacter are obligate anaerobes.

A composition of the invention includes a therapeutically effective amount of a bacterial strain of the invention. A therapeutically effective amount of a bacterial strain is sufficient to exert a beneficial effect upon a patient. A therapeutically effective amount of a bacterial strain may be sufficient to result in delivery to and/or partial or total colonisation of the patient's intestine.

A suitable daily dose of the bacteria, for example for an adult human, may be from about 1×10³ to about 1×10¹¹ colony forming units (CFU); for example, from about 1×10⁷ to about 1×10¹⁰ CFU; in another example from about 1×10⁶ to about 1×10¹⁰ CFU; in another example from about 1×10⁷ to about 1×10¹¹ CFU; in another example from about 1×10⁸ to about 1×10¹⁰ CFU; in another example from about 1×10⁸ to about 1×10¹¹ CFU.

In certain embodiments, the dose of the bacteria is at least 10⁹ cells per day, such as at least 10¹⁰, at least 10¹¹, or at least 10¹² cells per day.

In certain embodiments, a dose of the composition may comprise the bacterial strain in an amount of from about 1×10⁶ to about 1×10¹¹ colony forming units (CFU)/g, respect to the weight of the composition. The dose may be suitable for an adult human. For example, the composition may comprise the bacterial strain from about 1×10³ to about 1×10¹¹ CFU/g; for example, from about 1×10⁷ to about 1×10¹⁰ CFU/g; in another example from about 1×10⁶ to about 1×10¹⁰ CFU/g; in another example from about 1×10⁷ to about 1×10¹¹ CFU/g; in another example from about 1×10⁸ to about 1×10¹⁰ CFU/g; in another example from about 1×10⁸ to about 1×10¹¹ CFU/g, from about 1×10⁸ to about 1×10¹⁰ CFU/g. For example, from about 1×10⁸ to about 1×10¹⁰ CFU/g. The dose may be, for example, 1 g, 3 g, 5 g, and 10 g.

In some embodiments, the compositions described above and/or elsewhere herein comprise, consist, or consist essentially of an amount of bacterial strain from about 1×10³ to about 1×10¹¹ colony forming units per gram with respect to a weight of the composition.

In some embodiments, the compositions described above and/or elsewhere herein comprise the bacterial strain at a dose of between 500 mg and 1000 mg, between 600 mg and 900 mg, between 700 mg and 800 mg, between 500 mg and 750 mg or between 750 mg and 1000 mg. In certain embodiments, the invention provides the above pharmaceutical composition, wherein the lyophilised bacteria in the pharmaceutical composition is administered at a dose of between 500 mg and 1000 mg, between 600 mg and 900 mg, between 700 mg and 800 mg, between 500 mg and 750 mg, or between 750 mg and 1000 mg.

The composition may be formulated as a probiotic. A probiotic is defined by the FAO/WHO as a live microorganism that, when administered in adequate amounts, confers a health benefit on the host.

Typically, a probiotic, such as the composition of the invention, is optionally combined with at least one suitable prebiotic compound. A prebiotic compound is usually a non-digestible carbohydrate such as an oligosaccharide or polysaccharide, or a sugar alcohol, which is not degraded or absorbed in the upper digestive tract. Known prebiotics include commercial products such as inulin and transgalactoligosaccharides.

Other prebiotic compounds (such as vitamin C, for example), may be included as oxygen scavengers and to improve the delivery and/or partial or total colonisation and survival in vivo. Alternatively, the probiotic composition of the invention may be administered orally as a food or nutritional product, such as milk or whey based fermented dairy product, or as a pharmaceutical product.

In certain embodiments, the probiotic composition of the present invention includes a prebiotic compound in an amount of from about 1 to about 30% by weight, respect to the total weight composition (e.g., from 5 to 20% by weight). Known prebiotics include commercial products such as inulin and transgalactoligosaccharides.

In some embodiments, the prebiotic is a carbohydrate selected from the group comprising or consisting of fructooligosaccharides (or FOS), short-chain fructooligosaccharides, inulin, isomaltoligosaccharides, pectins, xylooligosaccharides (or XOS), chitosanoligosaccharides (or COS), beta-glucans, arable gum modified and resistant starches, polydextrose, D-tagatose, acacia fibers, carob, oats, and citrus fibers. In one aspect, the prebiotics are the short-chain fructooligosaccharides. Short-chain FOS are not digestible carbohydrates, generally obtained by the conversion of the beet sugar and including a saccharose molecule to which three glucose molecules are bonded.

The compositions of the invention may comprise pharmaceutically acceptable excipients or carriers, such as those described in Handbook of Pharmaceutical Excipients. Acceptable carriers or diluents for therapeutic use are well known in the pharmaceutical art and are described, for example, in Remington's Pharmaceutical Sciences. Examples of suitable carriers include lactose, starch, glucose, methyl cellulose, magnesium stearate, mannitol, sorbitol and the like. Examples of suitable diluents include ethanol, glycerol and water. The choice of pharmaceutical carrier, excipient or diluent can be selected with regard to the intended route of administration and standard pharmaceutical practice. The pharmaceutical compositions may comprise as, or in addition to, the carrier, excipient or diluent one or more suitable binders, lubricants, suspending agents, coating agents, and/or solubilising agents. Examples of suitable binders include starch, gelatin, natural sugars such as glucose, anhydrous lactose, free-flow lactose, R-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilizers, dyes and even flavouring agents may be provided in the pharmaceutical composition. Examples of preservatives include sodium benzoate, sorbic acid, cysteine and esters of 4-hydroxybenzoic acid, for example, in some embodiments the preservative is selected from sodium benzoate, sorbic acid and esters of 4-hydroxybenzoic acid. Antioxidants and suspending agents may be also used. A further example of a suitable carrier is saccharose. A further example of a suitable preservative is cysteine.

The compositions of the invention may be formulated as a food product. For example, a food product may provide nutritional benefit in addition to the therapeutic effect of the invention, such as in a nutritional supplement. Similarly, a food product may be formulated to enhance the taste of the composition of the invention or to make the composition more attractive to consume by being more similar to a common food item, rather than to a pharmaceutical composition. In certain embodiments, the composition of the invention is formulated as a milk-based product. The term “milk-based product” means any liquid or semi-solid milk-based or whey-based product having a varying fat content. The milk-based product can be, e.g., cow's milk, goat's milk, sheep's milk, skimmed milk, whole milk, milk recombined from powdered milk and whey without any processing, or a processed product, such as yoghurt, curdled milk, curd, sour milk, sour whole milk, butter milk and other sour milk products. Alternatively, the milk could be a plant-based milk, including for example, soy milk, oat milk, almond milk, coconut milk, or macadamia milk. Another important group includes milk beverages, such as whey beverages, fermented milks, condensed milks, infant or baby milks; flavoured milks, ice cream; milk-containing food such as sweets.

In some embodiments, the compositions disclosed herein comprise one or more bacterial strains of the genus Mediterraneibacter and do not contain bacteria from any other species, or which comprise only de minimis or biologically irrelevant amounts of bacteria from another species. Thus, in some embodiments, the invention provides a composition comprising one or more bacterial strains of the genus Mediterraneibacter, which does not contain bacteria from any other species or which comprises only de minimis or biologically irrelevant amounts of bacteria from another species, for use in therapy.

In some embodiments, the compositions comprise one or more bacterial strains of the genus Mediterraneibacter and do not contain bacteria from any other genus or comprise only de minimis or biologically irrelevant amounts of bacteria from another. In some embodiments, the compositions comprise one or more bacterial strains of the genus Mediterraneibacter and do not contain bacteria from any other genus or comprise only de minimis or biologically irrelevant amounts of bacteria from another.

In certain embodiments, the compositions disclosed herein contain a single bacterial species and do not contain any other bacterial species. In certain embodiments, the compositions disclosed herein contain a single bacterial strain and do not contain any other bacterial strains. For example, the compositions of the invention may comprise bacteria only of a strain of M. faecis. Such compositions may comprise only de minimis or biologically irrelevant amounts of other bacterial strains or species. Such compositions may be a culture that is substantially free from other species of organism. In some embodiments, such compositions may be a in a dried form and be substantially free from other species of organism.

In some embodiments, the invention provides a composition comprising a single bacterial strain of the genus Mediterraneibacter which does not contain bacteria from any other strains or which comprises only de minimis or biologically irrelevant amounts of bacteria from another strain for use in therapy.

In certain embodiments, the compositions of the invention contain a single bacterial strain or species and do not contain any other bacterial strains or species. Such compositions may comprise only de minimis or biologically irrelevant amounts of other bacterial strains or species. Such compositions may be a culture that is substantially free from other species of organism.

In certain embodiments, the compositions of the invention consist of 1, 2, 3, 4, 5, 6, 7, 8, 9, 10, 11, 12, 13, 14, 15 or 16 bacterial strains or species. In certain embodiments, the compositions consist of from 1 to 10, preferably from 1 to 5 bacterial strains or species. In some embodiments, the compositions disclosed herein comprise more than one strain from within the same species (e.g., more than 1, 2, 3, 4, 5, 6, 7, 8, 9, 10, 15, 20, 25, 30, 35, 40 or 45 strains), and, optionally, do not contain bacteria from any other species. In some embodiments, the compositions disclosed herein comprise less than 50 strains from within the same species (e.g., less than 45, 40, 35, 30, 25, 20, 15, 12, 10, 9, 8, 7, 6, 5, 4 or 3 strains), and, optionally, do not contain bacteria from any other species. In some embodiments, the compositions disclosed herein comprise 1-40, 1-30, 1-20, 1-19, 1-18, 1-15, 1-10, 1-9, 1-8, 1-7, 1-6, 1-5, 1-4, 1-3, 1-2, 2-50, 2-40, 2-30, 2-20, 2-15, 2-10, 2-5, 6-30, 6-15, 16-25, or 31-50 strains from within the same species and, optionally, do not contain bacteria from any other species. In some embodiments, the compositions disclosed herein comprise more than one species from within the same genus (e.g., more than 1, 2, 3, 4, 5, 6, 7, 8, 9, 10, 12, 15, 17, 20, 23, 25, 30, 35 or 40 species), and, optionally, do not contain bacteria from any other genus. In some embodiments, the compositions disclosed herein comprise less than 50 species from within the same genus (e.g., less than 50, 45, 40, 35, 30, 25, 20, 15, 12, 10, 8, 7, 6, 5, 4 or 3 species), and, optionally, do not contain bacteria from any other genus. In some embodiments, the compositions disclosed herein comprise 1-50, 1-40, 1-30, 1-20, 1-15, 1-10, 1-9, 1-8, 1-7, 1-6, 1-5, 1-4, 1-3, 1-2, 2-50, 2-40, 2-30, 2-20, 2-15, 2-10, 2-5, 6-30, 6-15, 16-25, or 31-50 species from within the same genus and, optionally, do not contain bacteria from any other genus. The invention comprises any combination of the foregoing.

In some embodiments, the compositions of the invention comprise more than one bacterial strain or species. For example, in some embodiments, the compositions of the invention comprise more than one strain from within the same species (e.g., more than 1, 2, 3, 4, 5, 6, 7, 8, 9, 10, 15, 20, 25, 30, 35, 40 or 45 strains), and, optionally, do not contain bacteria from any other species. In some embodiments, the compositions of the invention comprise less than 50 strains from within the same species (e.g., less than 45, 40, 35, 30, 25, 20, 15, 12, 10, 9, 8, 7, 6, 5, 4 or 3 strains), and, optionally, do not contain bacteria from any other species. In some embodiments, the compositions of the invention comprise 1-40, 1-30, 1-20, 1-19, 1-18, 1-15, 1-10, 1-9, 1-8, 1-7, 1-6, 1-5, 1-4, 1-3, 1-2, 2-50, 2-40, 2-30, 2-20, 2-15, 2-10, 2-5, 6-30, 6-15, 16-25, or 31-50 strains from within the same species and, optionally, do not contain bacteria from any other species. In some embodiments, the compositions of the invention comprise more than one species from within the same genus (e.g., more than 1, 2, 3, 4, 5, 6, 7, 8, 9, 10, 12, 15, 17, 20, 23, 25, 30, 35 or 40 species), and, optionally, do not contain bacteria from any other genus. In some embodiments, the compositions of the invention comprise less than 50 species from within the same genus (e.g., less than 50, 45, 40, 35, 30, 25, 20, 15, 12, 10, 8, 7, 6, 5, 4 or 3 species), and, optionally, do not contain bacteria from any other genus. In some embodiments, the compositions of the invention comprise 1-50, 1-40, 1-30, 1-20, 1-15, 1-10, 1-9, 1-8, 1-7, 1-6, 1-5, 1-4, 1-3, 1-2, 2-50, 2-40, 2-30, 2-20, 2-15, 2-10, 2-5, 6-30, 6-15, 16-25, or 31-50 strains from within the same genus and, optionally, do not contain bacteria from any other genus. The invention comprises any combination of the foregoing.

In certain embodiments, the pharmaceutical composition of the invention comprises between 1-50 distinct bacterial strains, such as between 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3 or 2 distinct bacterial strains. In certain embodiments, the pharmaceutical composition of the invention comprises between 1-50 distinct bacterial strains, such as between 1-50, 1-40, 1-30, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3 or 2 distinct bacterial strains.

In some embodiments, the composition of the invention additionally comprises a bacterial strain that has the same safety and therapeutic efficacy characteristics as any one of strains V21/006223, V21/006224, V21/006225, or V21/006226.

In some embodiments in which the composition of the invention comprises more than one bacterial strain, species or genus, the individual bacterial strains, species or genera may be for separate, simultaneous or sequential administration. For example, the composition may comprise all of the more than one bacterial strain, species or genera, or the bacterial strains, species or genera may be stored separately and be administered separately, simultaneously or sequentially. In some embodiments, the more than one bacterial strains, species or genera are stored separately but are mixed together prior to use.

Preferably, the compositions disclosed herein are to be administered to the gastrointestinal (GI) tract in order to enable delivery to, and/or partial or total colonisation of, the intestine with the bacterial strain of the invention. In other words, the bacteria may colonise some or all of the GI tract and such colonisation may be transient or permanent. More specifically, the phrase “total colonisation of the intestine” means that bacteria have colonised all parts of the intestine (i.e., the small intestine, large intestine and rectum). Additionally or alternatively, the term “total colonisation” means that the bacteria engraft permanently in some or all parts of the intestine.

Similarly, the phrase “partial colonisation of the intestine” means that bacteria have colonised some but not all parts of the intestine. Additionally or alternatively, the term “partial colonisation” means that the bacteria engraft transiently in some or all parts of the intestine.

The transience of engraftment of bacteria can be determined by assessing (e.g., in a fecal sample) the abundance of the bacterial strain of the invention periodically (e.g., daily or weekly) following the end of a dosing interval to determine the washout period, i.e., the period between conclusion of the dosing interval and there being no detectable levels of the bacterial strain of the invention present. In some embodiments, the washout period is 14 days or less, 12 days or less, 10 days or less, 7 days or less, 4 days or less, 3 days or less, 2 days or less, or 1 day or less.

In some embodiments, the bacteria described above or elsewhere herein engraft transiently in the large intestine.

In some embodiments, the bacterial strains of the invention are obtained from human adult faeces. In some embodiments in which the composition of the invention comprises more than one bacterial strain, all of the bacterial strains are obtained from human adult faeces or if other bacterial strains are present they are present only in de minimis amounts. The bacteria may have been cultured subsequent to being obtained from these human adult faeces and being used in a composition of the invention.

In some embodiments, the one or more Mediterraneibacter bacterial strain is/are the only therapeutically active agents in a composition of the invention. In some embodiments, the bacterial strains in the composition is/are the only therapeutically active agents in a composition of the invention.

The compositions for use in accordance with the invention may or may not require marketing approval.

In certain embodiments, the invention provides the above pharmaceutical composition, wherein said bacterial strain is in a dried form. In some cases, the bacterial strain is reconstituted prior to administration. In some cases, the reconstitution is by use of a diluent described herein. In certain embodiments, the invention provides the above pharmaceutical composition, wherein said bacterial strain is spray dried. In certain embodiments, the invention provides the above pharmaceutical composition, wherein the bacterial strain is lyophilised or spray dried and wherein it is live. In certain embodiments, the invention provides the above pharmaceutical composition, wherein the bacterial strain is lyophilised or spray dried and wherein it is viable. In certain embodiments, the invention provides the above pharmaceutical composition, wherein the bacterial strain is lyophilised or spray dried and wherein it is capable of partially or totally colonising the intestine. In certain embodiments, the invention provides the above pharmaceutical composition, wherein the bacterial strain is dried (e.g., lyophilised or spray dried) and wherein it is viable and capable of partially or totally colonising the intestine. In some of the same embodiments and some alternative embodiments, the bacterial strain transiently colonises the intestine.

In some cases, the lyophilised or spray dried bacterial strain is reconstituted prior to administration. In some cases, the reconstitution is by use of a diluent described herein.

The compositions of the invention can comprise pharmaceutically acceptable excipients, diluents or carriers.

In certain embodiments, the invention provides a pharmaceutical composition comprising: a bacterial strain of the invention; and a pharmaceutically acceptable excipient, carrier or diluent; wherein the bacterial strain is in an amount sufficient to treat or prevent an inflammatory or autoimmune disorder when administered to a subject in need thereof. In some preferred embodiments, the inflammatory or autoimmune disorder is selected from the group comprising: an inflammatory bowel disease (such as Crohn's disease or ulcerative colitis); asthma (such as allergic asthma or neutrophilic asthma); arthritis (such as rheumatoid arthritis, osteoarthritis, psoriatic arthritis, or juvenile idiopathic arthritis); fatty liver disease (such as nonalcoholic fatty liver disease (NAFLD)); ankylosing spondylitis; psoriasis; systemic lupus erythematosus (SLE); scleroderma; Sjogren's syndrome; vasculitis; type 1 diabetes mellitus.

In certain embodiments, the invention provides pharmaceutical composition comprising: a bacterial strain of the invention; and a pharmaceutically acceptable excipient, carrier or diluent; wherein the bacterial strain is in an amount sufficient to treat or prevent an inflammatory or autoimmune disorder mediated by the STAT3 signalling pathway. In preferred embodiments, said disorder is selected from the group consisting of an inflammatory bowel disease (such as Crohn's disease or ulcerative colitis); asthma (such as allergic asthma or neutrophilic asthma); arthritis (such as rheumatoid arthritis, osteoarthritis, psoriatic arthritis, or juvenile idiopathic arthritis); fatty liver disease (such as nonalcoholic fatty liver disease (NAFLD)); ankylosing spondylitis; psoriasis; systemic lupus erythematosus (SLE); scleroderma; Sjogren's syndrome; vasculitis; type 1 diabetes mellitus.

In certain embodiments, the invention provides the above pharmaceutical composition, wherein the amount of the bacterial strain is from about 1×10³ to about 1×10¹¹ colony forming units (CFU) per gram with respect to a weight of the composition.

In certain embodiments, the invention provides the above pharmaceutical composition, wherein the composition is administered at a dose of 1 g, 3 g, 5 g or 10 g.

In certain embodiments, the invention provides the above pharmaceutical composition, wherein the composition is administered by a method selected from the group consisting of oral, rectal, subcutaneous, nasal, buccal, and sublingual.

In certain embodiments, the invention provides the above pharmaceutical composition, comprising a carrier selected from the group consisting of lactose, starch, glucose, methyl cellulose, magnesium stearate, mannitol and sorbitol.

In certain embodiments, the invention provides the above pharmaceutical composition, comprising a diluent selected from the group consisting of ethanol, glycerol and water.

In certain embodiments, the invention provides the above pharmaceutical composition, comprising an excipient selected from the group consisting of starch, gelatin, glucose, anhydrous lactose, free-flow lactose, beta-lactose, corn sweetener, acacia, tragacanth, sodium alginate, carboxymethyl cellulose, polyethylene glycol, sodium oleate, sodium stearate, magnesium stearate, sodium benzoate, sodium acetate and sodium chloride.

In certain embodiments, the invention provides the above pharmaceutical composition, further comprising at least one of a preservative, an antioxidant and a stabilizer.

In certain embodiments, the invention provides the above pharmaceutical composition, comprising a preservative selected from the group consisting of sodium benzoate, sorbic acid and esters of 4-hydroxybenzoic acid.

In certain embodiments, the invention provides the above pharmaceutical composition, wherein said bacterial strain is in a dried form (e.g., lyophilized, spray dried, fluidized bed dried, etc.).

In certain embodiments, the invention provides the above pharmaceutical composition, wherein when the composition is stored in a sealed container at about 4° C. or about 25° C. and the container is placed in an atmosphere having 50% relative humidity, at least 80% of the bacterial strain as measured in colony forming units, remains after a period of at least about: 1 month, 3 months, 6 months, 1 year, 1.5 years, 2 years, 2.5 years or 3 years.

In some embodiments, the composition of the invention is provided in a sealed container comprising a composition as described herein. In some embodiments, the sealed container is a sachet or bottle. In some embodiments, the composition of the invention is provided in a syringe comprising a composition as described herein.

The composition of the present invention may, in some embodiments, be provided as a pharmaceutical formulation. For example, the composition may be provided as a tablet or capsule. In some embodiments, the capsule is a gelatine capsule (“gel-cap”). The capsule can be a hard or a soft capsule. In some embodiments, the formulation is a soft capsule. Soft capsules are capsules which may, owing to additions of softeners, such as, for example, glycerol, sorbitol, maltitol and polyethylene glycols, present in the capsule shell, have a certain elasticity and softness. Soft capsules can be produced, for example, on the basis of gelatine or starch. Gelatine-based soft capsules are commercially available from various suppliers. Depending on the method of administration, such as, for example, orally or rectally, soft capsules can have various shapes, they can be, for example, round, oval, oblong or torpedo-shaped. Soft capsules can be produced by conventional processes, such as, for example, by the Scherer process, the Accogel process or the droplet or blowing process.

In some embodiments, the compositions disclosed herein are administered orally. Oral administration may involve swallowing, so that the compound enters the GI tract.

Pharmaceutical formulations suitable for oral administration include solid plugs, solid microparticulates, semi-solid and liquid (including multiple phases or dispersed systems) such as tablets; soft or hard capsules containing multi- or nano-particulates, liquids (e.g., aqueous solutions), emulsions or powders; lozenges (including liquid-filled); chews; gels; fast dispersing dosage forms; films; ovules; sprays; and buccal/mucoadhesive patches.

In some embodiments the pharmaceutical formulation is an enteric formulation, i.e., a gastro-resistant formulation (for example, resistant to gastric pH) that is suitable for delivery of the composition of the invention to the intestine by oral administration. Enteric formulations may be particularly useful when the bacteria or another component of the composition is acid-sensitive (e.g., prone to degradation under gastric conditions).

In some embodiments, the enteric formulation comprises an enteric coating. In some embodiments, the formulation is an enteric-coated dosage form. For example, the formulation may be an enteric-coated tablet or an enteric-coated capsule, or the like. The enteric coating may be a conventional enteric coating, for example, a conventional coating for a tablet, capsule, or the like for oral delivery. The formulation may comprise a film coating, for example, a thin film layer of an enteric polymer (e.g., an acid-insoluble polymer).

In some embodiments, the enteric formulation is intrinsically enteric, for example, gastro-resistant without the need for an enteric coating. Thus, in some embodiments, the formulation is an enteric formulation that does not comprise an enteric coating. In some embodiments, the formulation is a capsule made from a thermogelling material. In some embodiments, the thermogelling material is a cellulosic material, such as methylcellulose, hydroxymethylcellulose or hydroxypropylmethylcellulose (HPMC). In some embodiments, the capsule comprises a shell that does not contain any film forming polymer. In some embodiments, the capsule comprises a shell and the shell comprises hydroxypropylmethylcellulose and does not comprise any film-forming polymer (as described in United States Patent Publication No. 2016/0067188). In some embodiments, the formulation is an intrinsically enteric capsule (for example, VCAPS® from Capsugel).

In some embodiments, the composition is a probiotic or a medical food comprising a bacterial strain of M. faecis. The bacteria can be administered, for instance, as a probiotic, as a capsule, tablet, caplet, pill, troche, lozenge, power, and/or granule. This strain can also be formulated as a nutraceutical, conventional food, medical food, or drug. The bacteria can also be administered as part of a fecal transplant or via suppository. In some embodiments, the composition is formulated for delivery to the gut, as described further herein, in some embodiments had the composition further comprise a prebiotic.

6.1 Co-Administering with Additional Agents

In some embodiments, the methods described herein can further comprise co-administering a second agent and/or treatment to the subject (e.g., as part of a therapy). The combination therapy, where employed, an be tailored to the particular indication. For example, where a strain of the species M. faecis is administered to treat an inflammatory disorder (e.g., an inflammatory bowel disease), it can be administered in combination with an anti-inflammatory agent or therapy as known in the art of approved for clinical treatment of an inflammatory disorder. Other indications can be similarly treated with, for example, strains of the species M. faecis as described herein in combination with agents known in the art or approved for the clinical treatment of those indications.

Suitable anti-inflammatory agents that could be used in the treatment of an inflammatory bowel disease include, but not necessarily limited are, the group comprising 5-aminosaliculates, corticosteroids, azathioprine, infliximab, and adalimumab.

The present invention also includes the compositions as described above, further comprising an anti-inflammatory agent. Such compositions can optionally be in the form of a single composition, or alternatively, two of more separate compositions.

### 7. Screening Methods

The invention also includes methods of identifying bacterial strains that are suitable for use in the methods of the present invention. Such methods typically include screening for a bacterial strain with a particular functional activity. Suitable assays include those described in the below examples, but any assay for measuring gut barrier function, mucosal healing, NFkB suppression, or inhibition of STAT3 signalling are equally as applicable.

In some embodiments, the screening method identifies the ability of a bacterial strain of Mediterraneibacter to inhibit or suppress STAT3 signalling pathway. By way of an illustrative example, the invention provides a method of blocking or otherwise inhibiting the activation of STAT3 signalling in a target cell, the method comprising contacting the target cell with at least a soluble component of a bacterial cell preparation of the species Mediterraneibacter faecis, to block or otherwise inhibit the activation of STAT3 signalling in the target cell.

In some embodiments of this type, the target cell is selected from the group comprising screening a bacterial strain for a functional aca reporter cell (e.g., a HEK cell), an immune cell (e.g., a Th17 immune cell), an epithelial cell, and an endothelial cell.

In some embodiments, the bacterial cell preparation comprises a bacterial cell culture. Suitably, the soluble component may comprise the supernatant of the bacterial cell culture. In some embodiments of this type, the soluble component is substantially depleted of bacterial cells.

In some alternative embodiments, the bacterial cell preparation comprises a bacterial cell pellet. Preferably, the bacterial cells of the cell pellet are lysed by any means known in the art. After cell lysis, it is typical for the cell lysate soluble fraction to be separated from the insoluble fraction. The cell lysate may be subject to further processing before being during the screening assay. (e.g., diluted in a buffer), or exposed to a processing reagent.

### 8. Modes of Administration

Preferably, the compositions of the invention are to be administered to the GI tract in order to enable delivery to the intestine with the bacterial strain of the invention. referable, the compositions of the invention are formulated to be administered to the GI tract in order to enable delivery to the intestine with the bacterial strain of the invention. In some embodiments the compositions of the invention are formulated to be administered to the GI tract in order to enable delivery to, and partial or total colonization of, the intestine with the bacterial strain of the invention.

In certain embodiments, the compositions of the invention may be 35 administered as a foam, as a spray or a gel.

In certain embodiments, the compositions of the invention may be administered as a suppository, such as a rectal suppository, for example in the form of a Theobroma oil (cocoa butter), synthetic hard fat (e.g., Suppocire®, WITEPSOL), glycerogelatin, polyethylene glycol, or soap glycerin composition.

In certain embodiments, the compositions of the invention are administered to the GI tract via a tube, such as a nasogastric tube, orogastric tube, gastric tube, jejunostomy tube (J-tube), percutaneous endoscopic gastrostomy (PEG), or a port, such as a chest wall port that provides access to the stomach, jejunum and other suitable access ports.

The compositions of the invention may be administered once, or they may be administered sequentially as part of a treatment regimen. In certain embodiments, the compositions of the invention are to be administered daily (either once or several times). In certain embodiments, the compositions disclosed herein are administered regularly, such as daily, every two days, or weekly, for an extended period of time, such as for at least one week, two weeks, one month, two months, six months, or one year.

In some embodiments, the compositions disclosed herein are administered for 7 days, 14 days, 16 days, 21 days or 28 days or no more than 7 days, 14 days, 16 days, 21 days, or 28 days. For example, in some embodiments the compositions disclosed herein are administered for 16 days.

In certain embodiments of the invention, treatment according to the invention is accompanied by assessment of the patient's gut microbiota. Treatment may be repeated if delivery of and/or partial or total colonisation with the strain of the invention is not achieved such that efficacy is not observed, or treatment may be ceased if delivery and/or partial or total colonisation is successful and efficacy is observed.

In certain embodiments, the composition of the invention may be administered to a pregnant animal, for example a mammal such as a human in order to prevent an inflammatory or autoimmune disorder (such as those disclosed herein) developing in her child in utero and/or after it is born.

The compositions of the invention may be administered to a patient that has been diagnosed with: a disease or condition mediated by the STAT3 signalling pathway, or that has been identified as being at risk of a disease or condition mediated by the STAT3 signalling pathway; or an inflammatory or autoimmune disorder (such as those disclosed herein). The compositions may also be administered as a prophylactic measure to prevent the development of diseases or conditions mediated by the STAT3 signalling pathway in a healthy patient.

The compositions disclosed herein may be administered to a patient that has been diagnosed with an inflammatory or autoimmune disorder, in particular an inflammatory or autoimmune disorder mediated by the microbiota-gut axis, or that has been identified as being at risk of an inflammatory or autoimmune disorder, in particular an inflammatory or autoimmune disorder mediated by the microbiota-gut axis. The compositions may also be administered as a prophylactic measure to prevent the development of inflammatory or autoimmune disorders, in particular inflammatory or autoimmune disorders mediated by the microbiota-gut axis in a healthy patient.

The compositions of the invention may be administered to a patient that has been identified as having an abnormal gut microbiota. For example, the patient may have reduced or absent colonisation by Mediterraneibacter, in particular M. faecis.

The compositions of the invention may be administered as a food product, such as a nutritional supplement.

Generally, the compositions of the invention are for the prevention or treatment of human diseases, although they may be used to treat animals including monogastric mammals such as poultry, pigs, cats, dogs, horses or rabbits. The compositions of the invention may be useful for enhancing the growth and performance of animals. If administered to animals, oral gavage may be used.

In some embodiments, the subject to whom the composition is to be administered is an adult human. In some embodiments, the subject to whom the composition is to be administered is an infant human.

### 9. Culturing Methods

The bacterial strains for use in the present invention can be cultured using standard microbiology techniques as detailed in, for example, references (Handbook of Microbiological Media, 2010; Hunter-Cevera, 1996).

The solid or liquid medium used for culture may, for example, be selected from TY or PYG medium.

Illustrative media formulations that are suitable for use with the present invention include those provided in Table 1.

TABLE 1
Culture Media Formulations

| | TY | | PYG | | YG/V | |
|---|---|---|---|---|---|---|
| Tryptone | 10 | g | 20 | g | — | |
| Vegetable tryptone | | | | | 20 | g |
| Yeast extract | 2.5 | g | Tabffff10 g | | 10 | g |
| Glucose | 4 | g | 10 | g | 10 | g |
| Cellobiose | 1 | g | — | | — | |
| Maltose | 1 | g | — | | — | |
| Hemin solution | 10 | mL | 1 | mL | — | |
| Acetic acid | 1.9 | mL | — | | — | |
| Salts 2* | 38 | mL | 38 | mL | 38 | g |
| Salt 3* | 38 | mL | 38 | mL | 38 | g |
| Sodium bicarbonate | 8 | g | 8 | g | 8 | g |
| Resazurin | 1 | mL | 1 | mL | 1 | mL |
| Cysteine | 1 | g | 1 | g | 1 | g |

| | | | |
|---|---|---|---|
| Water | to 1000 mL | to 1000 mL | to 1000 mL |

*Salts 2: K₂HPO₄ 6 g/L
*Salts 3: KH₂PO₄ 6 g/L, (NH₄)2SO₄ 6 g/L, NaCl 12 g/L, MgSO₄•7H₂O 2.5 g/L, CaCl₂•2H₂O 1.6 g/L
Resazurin Stock 1000x (0.1%): 100 mg in 100 mL H₂O. See McSweeney et al. for further recipe details.

In order that the invention may be readily understood and put into practical effect, particular preferred embodiments will now be described by way of the following non-limiting experimental examples.

### EXAMPLES

Mediterraneibacter Association with Health, IBD and Other Diseases

Inflammatory bowel disease is characterised by structure-function changes to the microbiome with a significant reduction in both the prevalence and abundance of select gut bacteria in the IBD gut when compared to the healthy gut. Several studies have shown that these bacteria may modulate IBD pathogenesis (Mallone et al., 2011; and Sokol et al., 2008) however a key obstacle to using these bacteria to develop new therapeutics has been that low resolution 16S rRNA based profiling does not provide sufficient resolution to accurately discriminate against health and IBD associated strains at a low taxonomic level (i.e., genus, species, strain).

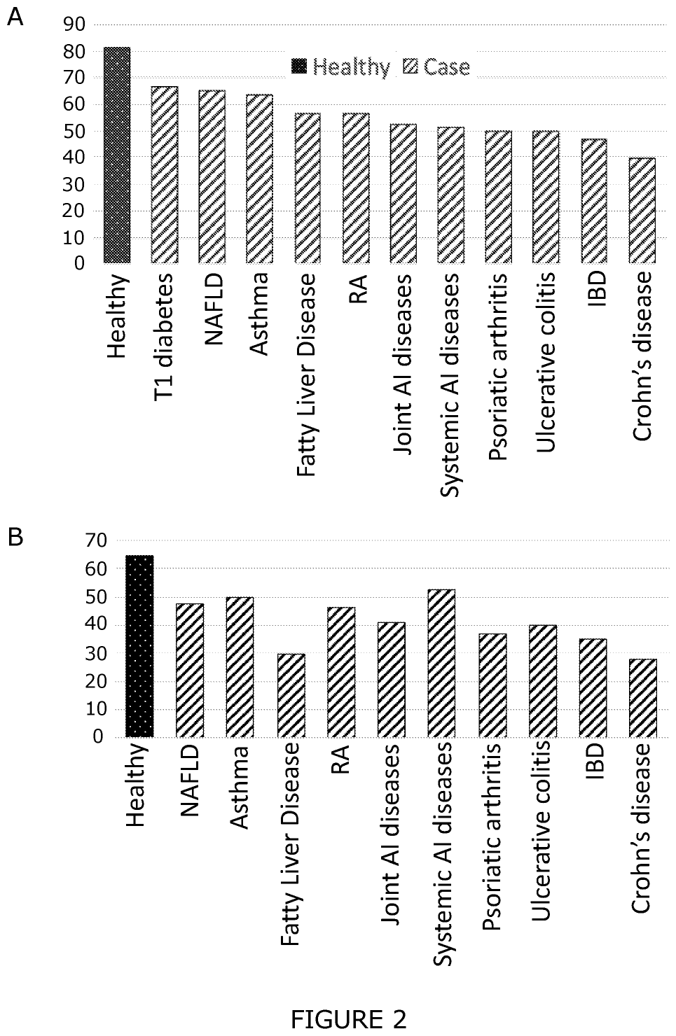

Using the Microba Discovery Database (MDD), which contains high resolution gut metagenomic data and associated host metadata for >8,000 subjects, we identified M. faecis and M. lactaris as a being prevalent in healthy humans but rarely detected in inflammatory and autoimmune diseases (FIG. 2, and Table 2). The strongest effect was observed for IBD, including both major subtypes ulcerative colitis and Crohn's disease (FIG. 2).

TABLE 2
M. faecis/M. lactaris Associations With Disorders

| | P-value | P-value |
|---|---|---|
| Disorder | M. faecis | M. lactaris |
| Asthma | 1.10 × 10⁻⁵ | 0.00094 |
| Autoimmune conditions affecting the | 6.50 × 10⁻⁵ | 0.00078 |
| joints (e.g., ankylosing spondylitis) | | |
| Psoriatic arthritis | 4.60 × 10⁻⁵ | 0.0013 |
| Rheumatoid arthritis | 2.90 × 10⁻⁵ | 0.0065 |
| Fatty live | 4.50 × 10⁻⁵ | 1.50 × 10⁻⁷ |
| IBD | 1.90 × 10⁻⁵ | 2.40 × 10⁻⁸ |
| Crohn's disease | 2.50 × 10⁻⁵ | 4.70 × 10⁻⁶ |
| Ulcerative colitis | 2.40 × 10⁻⁵ | 5.80 × 10⁻⁵ |
| Non-alcohol fatty liver disease | 0.022 | 0.038 |
| Systemic autoimmune disease (e.g., | 6.40 × 10⁻⁵ | 0.018 |
| lupus, scerloderma, Sjogren's | | |
| syndrome, vasculitis) | | |

Isolation and Genome-Scale Analysis of M. faecis.

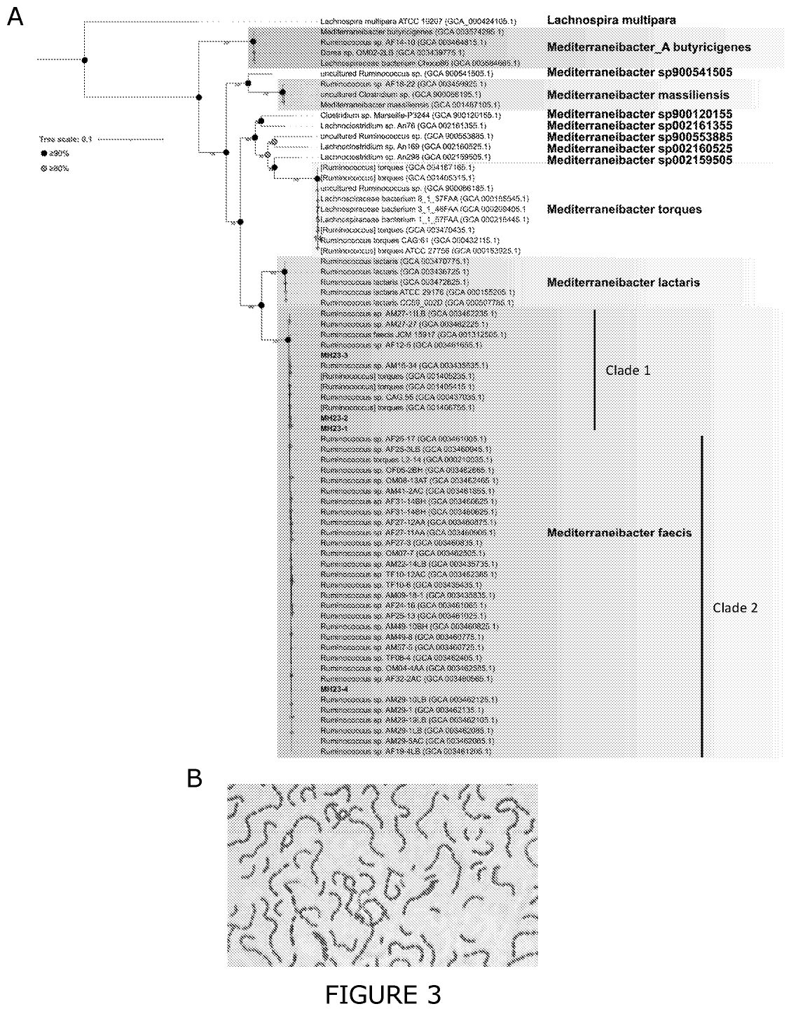

The four MH23 isolates place within the Mediterraneibacter genus, which combines several previously characterised isolates on the basis of average nucleotide identity (Togo et al., 2018). The phylogeny and functional potential of M. faecis was examined using high-quality publicly available genomes of Mediterraneibacter sp. from the Genome Taxonomy Database (GTDB; gtdb.ecogenomic.org (FIG. 3). M. faecis is currently the best represented species of the genus Mediterraneibacter with 40 high quality genomes and MAGs available at GTDB. It forms a distinct cluster with Mediterraneibacter lactaris and is separate from Mediterraneibacter torques and several other uncultured species (FIG. 3A). Interestingly, the M. faecis genomes cluster into two clades indicating this species is split into two subgroups. Metabolic reconstruction of M. faecis genomes revealed the ability to produce the short chain fatty acids propionate, lactate, acetate, and formate, but not butyrate (Table 3). A diverse profile of CAZymes were shared among the isolates suggesting a key role in fibre degradation. M. faecis is also predicted to use a wide range of monosaccharides as carbon sources (Table 3), including rhamnose which is relatively rare in the human gut microbiome. Interestingly, M. faecis is also predicted to synthesise selenocysteine in addition to the remaining 20 proteinogenic amino acids.

TABLE 3
Metabolic Reconstruction Analysis of SCFA Production

| | M. faecis | | | |
|---|---|---|---|---|
| SCFA | MH23-1 | MH23-2 | MH23-3 | MH23-4 |
| Formate | + | + | + | + |
| Acetate | + | + | + | + |
| Propionate | + | + | + | + |
| Butyrate | − | − | − | − |
| Isobutyrate | − | − | − | − |
| Valerate | − | − | − | − |
| Isovalerate | − | − | − | − |
| 2-Methylbutanoate | − | − | − | − |
| Lactate | + | + | + | + |

TABLE 4
Predictive Saccharide Carbon Sources of M. faecis Strains

| | | M. faecis strain | | | |
|---|---|---|---|---|---|
| | | 1 | 2 | 3 | 4 |
| Monosaccharides | Glucose | + | + | + | + |
| | L-Rhamnose | + | + | + | + |
| | Fructose | + | + | + | + |
| | D-Galactose | + | + | + | + |
| | D-Gluconate | + | + | + | + |
| | N-Acetylglucosamine | + | + | + | + |
| | D-Galacturonate | − | − | − | + |
| Disaccharides | Trehalose | + | + | + | + |
| | Lactose | + | + | + | + |
| | Melibiose | + | + | + | + |
| | Sucrose | + | + | + | + |
| | D-Mannopyranosyl-N- | − | − | − | + |
| | acetyl-D-glucosamine | | | | |
| | Laminaribiose degradation | − | − | + | + |
| Polysaccharides | Starch | + | + | + | + |
| | Glycogen | + | + | + | + |

To better understand the role of M. faecis in health and the pathogenesis of IBD, four new strains, termed M. faecis MH23-1, MH23-2, MH23-3, and MH23-4, were isolated from three healthy human donors by generating dilution-to-extinction enrichments and then plating for single colonies. All strains grew well on TY and PYG medium and were observed as Gram positive and occasionally Gram variable staining chain-forming cocci (FIG. 3B). Comparative genomic analyses revealed that the M. faecis MH23 strains clustered with high confidence (100% bootstrap support) within the M. faecis species. Both M. faecis MH23-1 and MH23-2 are highly similar with minor differences in gene composition and synteny suggesting a shared evolutionary history. In contrast, both M. faecis MH23-3 and MH23-4 differed from each other and both M. faecis MH23-1 and MH23-2 (FIG. 3A). M. faecis MH23-1, MH23-2 and MH23-3 were affiliated with cluster 1 while M. faecis MH23-4 was affiliated with cluster 2. As expected, the isolates were predicted to utilise glucose, fructose and N-acetylglucosamine which was consistent with the enrichment media used. All M. faecis MH23 isolates were predicted to produce B12 a relatively rare feature among the Firmicutes (Shelton et al., 2019).

M. faecis MH23-1 Improves Out Barrier Function In Vivo.

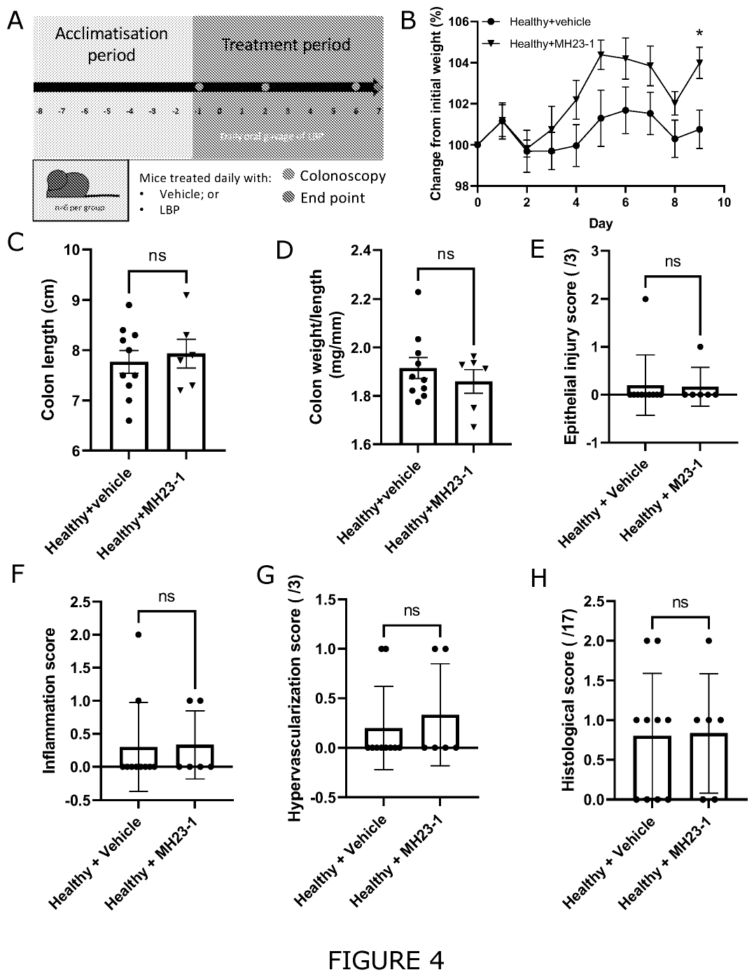

To assess the role of M. faecis in the healthy gut, naïve C57Bl/6 SPF mice were treated for 8 days with M. faecis MH23-1 (FIG. 4A). During this treatment period, no morbidity or change in general appearance, behaviour, posture, mobility and neurological behaviour was observed. Similarly, there was no significant change in body weight in M. faecis MH23-1 treated animals relative to the vehicle control; colon length and weight/length ratio were also unaffected (FIGS. 4B-4D). M. faecis MH23-1 did not result in any significant histological changes in the colon when compared to the vehicle as determined by assessing epithelial injury, inflammation and hypervascularization alone, or as a combined histopathological score (FIGS. 4E-4H).

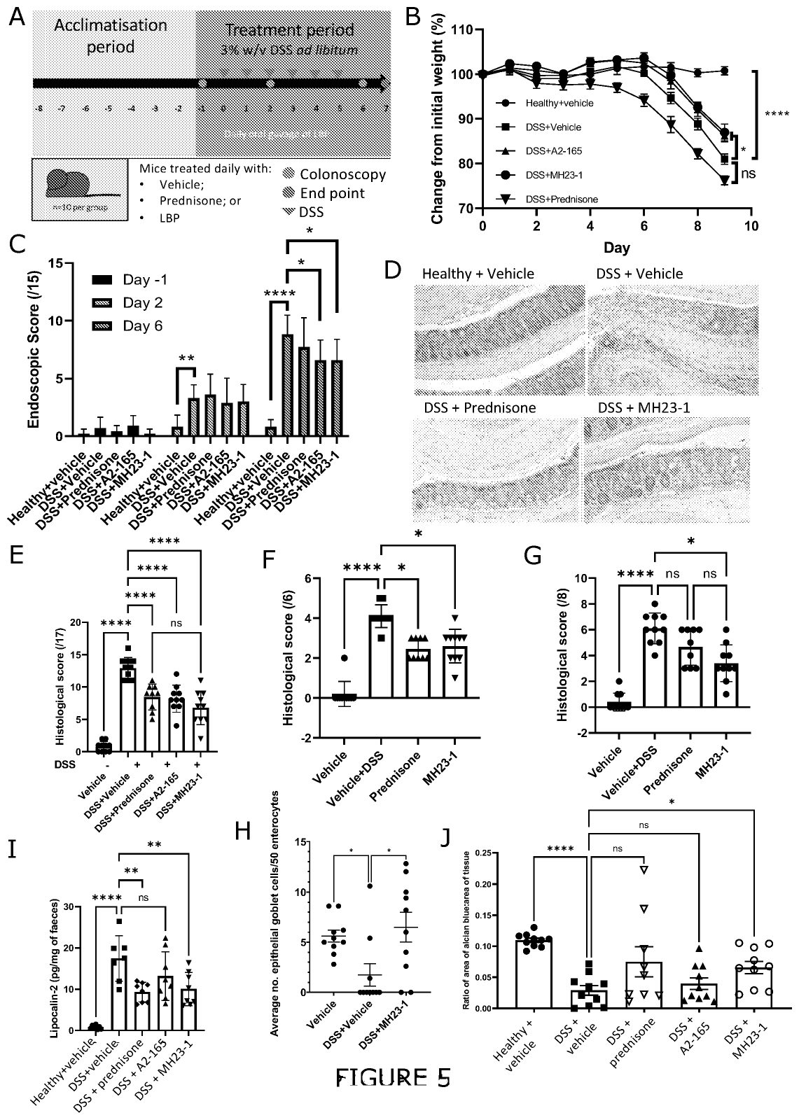

The therapeutic efficacy of M. faecis MH23-1 in an acute murine model of DSS-induced gut barrier dysfunction was examined, with prednisone and F. prausnitzii A2-165 as positive controls (FIG. 5A). DSS treatment resulted in significant gut barrier dysfunction relative to the vehicle control. Furthermore, there was a significant reduction in body weight (FIG. 5B) which has been shown to be an accurate and reliable indicator of gut barrier function (Britto et al., 2019). As expected, prednisone exacerbated the DSS induced weight loss (Yamamoto et al., 2013), however DSS-induced weight loss was ameliorated by treatment with M. faecis MH23-1 or F. prausnitzii A2-165 (FIG. 5B). Endoscopic analysis revealed a progressive increase in disease activity at day 2 and day 6 in all treatment groups, from baseline at day 1. However, treatment with F. prausnitzii A2-165 or M. faecis MH23-1 resulted in a significant reduction in disease activity relative to the vehicle treatment group (FIG. 5C).

Histological analysis of DSS-treated mice revealed significant gut damage characterised by crypt loss, epithelial erosion and ulceration. Notably, treatment with M. faecis MH23-1 resulted in significant improvement in pathology characterised by crypt re-formation and re-epithelisation (FIG. 5D) as evidenced by improvements in histopathological healing (FIG. 5D,E), epithelial injury and inflammation score (FIG. 5E-G). Consistent with this, treatment with M. faecis MH23-1 resulted in decreased gut inflammation as determined by lipocalin-2 in faeces (FIG. 5H). Histological analyses revealed an increase in epithelial goblet cells following M. faecis MH23-1 treatment (FIG. 5I) and this was associated by an increase in mucin production relative to the DSS treated control as determined by Alcian blue staining (FIG. 5J). As expected, prednisone and F. prausnitzii A2-165 also resulted in a significant improvement in disease pathology

Taken together, these data reveal that M. faecis MH23-1 did not cause any adverse effects in DSS treated or naïve mice, and that M. faecis MH23-1 promoted an improvement in gut barrier function and mucosal healing in as little as two days after the final DSS administration.

The inventors also examined the efficacy of M. faecis MH23-3 in a therapeutic model of DSS induced murine colits (FIG. 5K). Endoscopic analysis revealed treatment with prednisone or M. faecis MH23-1 resulted in a significant reduction in disease activity relative to the vehicle treatment group (FIG. 5L). Histological analysis of DSS-treated mice revealed significant gut damage characterised by crypt loss, epithelial erosion and ulceration. Notably, treatment with M. faecis MH23-3 resulted in significant improvement in pathology characterised by crypt re-formation and re-epithelisation as evidenced by improvements in histopathological healing, epithelial injury and inflammation score (FIG. 5M-0). Consistent with this, treatment with M. faecis MH23-3 resulted in decreased gut inflammation as determined by lipocalin-2 in faeces (FIG. 5P)

The therapeutic efficacy of M. faecis MH23-3 in an acute murine model of TNBS-induced colitis was also examined, with cyclosporine A as a positive control (FIG. 5Q). TNBS treatment resulted in significant histological damage that was ameliorated by treatment with cyclosporine A or M. faecis MH23-3 (FIG. 5R-S).

M. faecis Suppresses STAT3 and NF-κB Activation In Vitro.

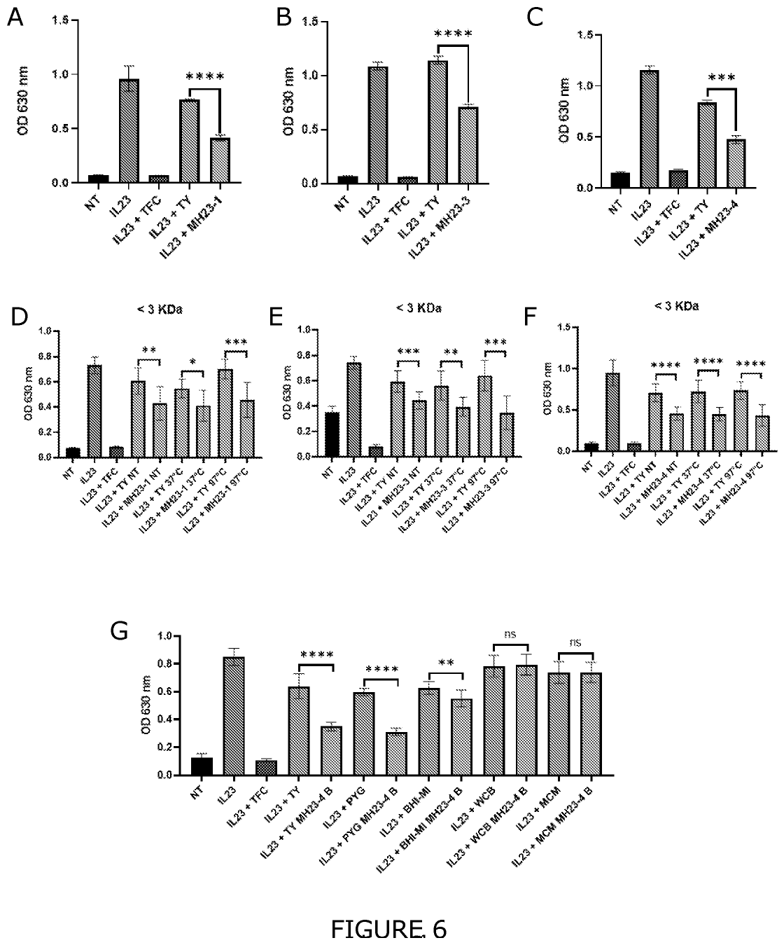

Given the dramatic effects on histological inflammation and re-epithelialisation observed in the DSS treated animals, the present inventors next examined the ability of M. faecis to modulate IBD associated immune pathways. IL-23 driven immune responses are central to the pathogenesis of IBD and are a clinically recognised target (Britto et al., 2019; and Yamamoto et al., 2013). The ability of M. faecis MH23-1, MH23-3 and MH23-4 to suppress IL-23 mediated activation of STAT3 was examined using the HEK-Blue™ IL-23 reporter cell line. The HEK-Blue™ IL-23 reporter cell line carries a STAT3 inducible SEAP reporter gene that is responsive to IL-23 stimulation. As expected, IL-23 mediated activation of STAT3 could be prevented by tofacitinib (FIG. 6A-C). Cell free culture supernatant prepared from M. faecis MH23-1, MH23-3, and MH23-4 grown in TY medium suppressed SEAP reporter activity (FIG. 6A-C). No cytotoxic effects were observed following treatment with M. faecis culture supernatants. The present inventors assessed the biochemical characteristics of the culture supernatant (CS) by size fractionation, heat treatment and proteinase K treatment. By this approach, it was determined that the STAT3 suppressive activity of M. faecis was associated with the <3 kDa fraction and it was unaffected by heat treatment (FIG. 6D-F).

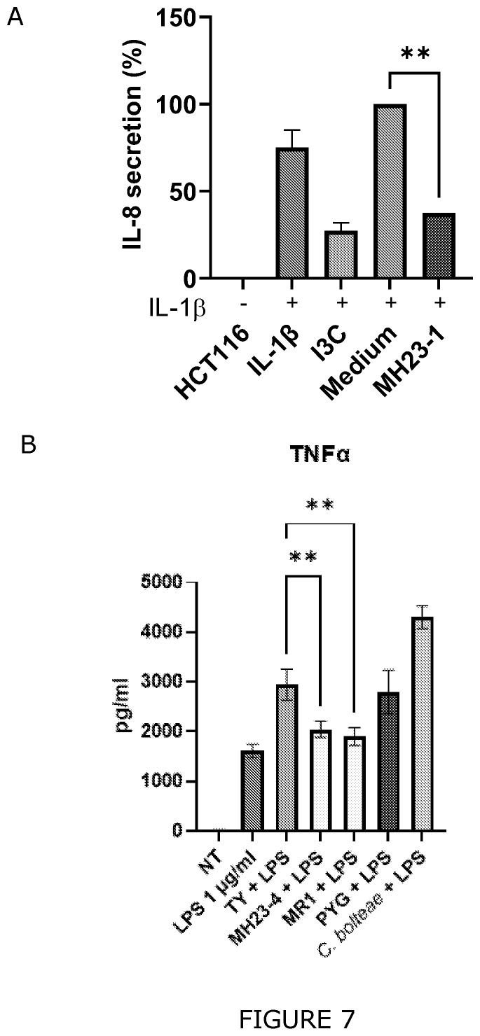

Finally, we examined the impact of growth medium dependent effects on IL-23 mediated STAT3 activation. CS prepared from strains grown in TY or PYG medium exhibited potent STAT3 suppressive activities. There was minimal STAT3 suppressive activity when the strains were grown in BHI, Wilkins-Chalgren medium (WCB) or MCM (FIG. 7G). This is consistent with previous reports on the influence of nutritional factors in culture impacting on activity for other bacterial species (e.g., Giri et al., 2019; and Toshimitsu et al., 2017). These results therefore suggest, that the production of immunomodulatory bioactives by M. faecis can be enhanced by select nutritional factors, as previously described for other bacterial species (Wlodarska et al., 2017; Zelante et al., 2013).

The ability of M. faecis MH23-1 and MH23-2 to prevent NF-κB activation was examined by assessing IL-8 secretion in the human gut epithelial HCT116 cell line (Kunsch et al., 1993). IL-8 expression is NF-κB regulated (Zhu et al., 2021) and as expected, treatment of HCT116 cells with IL-1p resulted in a significant increase in IL-8 secretion that could be prevented using the pharmacological inhibitor indole-3-carbinol (FIG. 7A). Notably, cell free CS from M. faecis MH23-1 suppressed IL-8 secretion relative to the medium control (FIG. 7A). Additionally, the ability of M. faecis MH23-4 to suppress NF-κB activity was tested by assessing the expression of TNF in human monocyte-derived macrophages. THP-1 cells were co-stimulated with LPS and cell free culture supernatant from M. faecis MH23-4. Both M. faecis MH23-4 and F. prausnitzii A2-165 were able to suppress the expression of TNF (FIG. 7B). In contrast, cell free CS prepared from Clostridium bolteae BAA-613 (a representative strain from an IBD-associated species (Lloyd-Price, 2019)) did not suppress TNF expression further underlying the substantial anti-inflammatory activity of M. faecis. Taken together, this revealed that M. faecis modulates activation of key pathways underpinning the IBD inflammatory response.

M. faecis Modulates GPCR Activity.

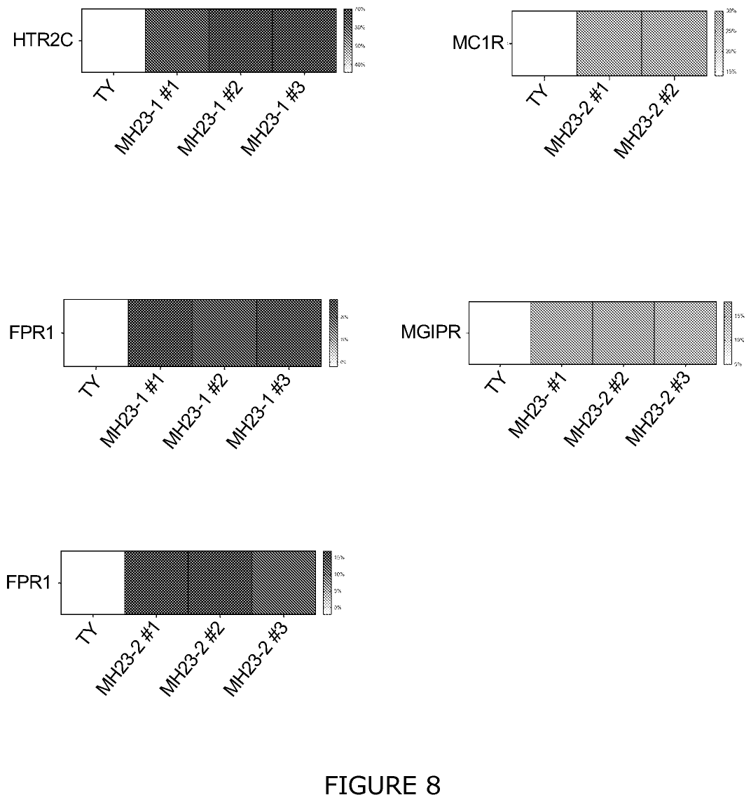

To better understand the ability of M. faecis to induce mucosal healing, the immunomodulatory capacities of M. faecis MH23-1 and MH23-2 were assessed using the high-throughput gpcrMAX℠ GPCR Assay Panel and SelectScreen Cell-based Pathway Profiling assays. The gpcrMAX℠ and SelectScreen assays are comprised of 168 and 38 pathways, respectively. To minimise the number of samples to be screened, a crude metabolite extract was prepared using a method adapted from Colosimo et al. (Colosimo et al., 2019) to extract low molecular weight non-polar metabolites. Uninoculated control medium was similarly prepared, and each sample was assayed in agonist and antagonist mode and confident hits identified using the criteria outlined by Eurofins. In the first pass screens, M. faecis MH23-1 and MH23-2 induced β-arrestin recruitment above the threshold level and differed significantly from the medium control. In particular, M. faecis MH23-1 and MH23-2 exhibited agonist activities against the GPCRs FPR1 and HTR2C, which are receptors for N-formylated peptides and tryptamine respectively, relative to the control medium. In addition, both strains exhibited antagonist activities against dopamine mediated activation of DRD2S, while M. faecis MH23-1 additionally exhibited antagonist activities against isoproterenol mediated activation of ADRB1 and dopamine mediated activation of DRD3. As crude metabolite extracts could reduce the sensitivity of the assays (e.g., Colosimo et al., 2019), hits that did not meet the stringent threshold but that differed from the medium control were also examined. From this extended list, several other GPCRs that are potentially modulated by M. faecis were identified. High and medium confidence hits were subsequently assessed with biological replicate cultures and confirmed that M. faecis strains MH23-1 and MH23-2 both activated FRP1 at the medium confidence threshold and that M. faecis MH23-1 additionally activated HTR2C (FIG. 7). Similarly, M. faecis MH23-2 exhibited antagonist activities against melanotan II mediated activation of MC1R and GIP mediated activation of GIPR at the medium confidence threshold (FIG. 8).

Separately, using the Pathway Hunter assays we determined that M. faecis MH23-1 and MH23-2 both suppress neurotrophin-3 activation of nuclear factor of activated T-cells (NFAT) in a reporter cell line.

M. faecis Modulates Cytokines in PBMC CD3⁺ and CD3⁻ Cells.

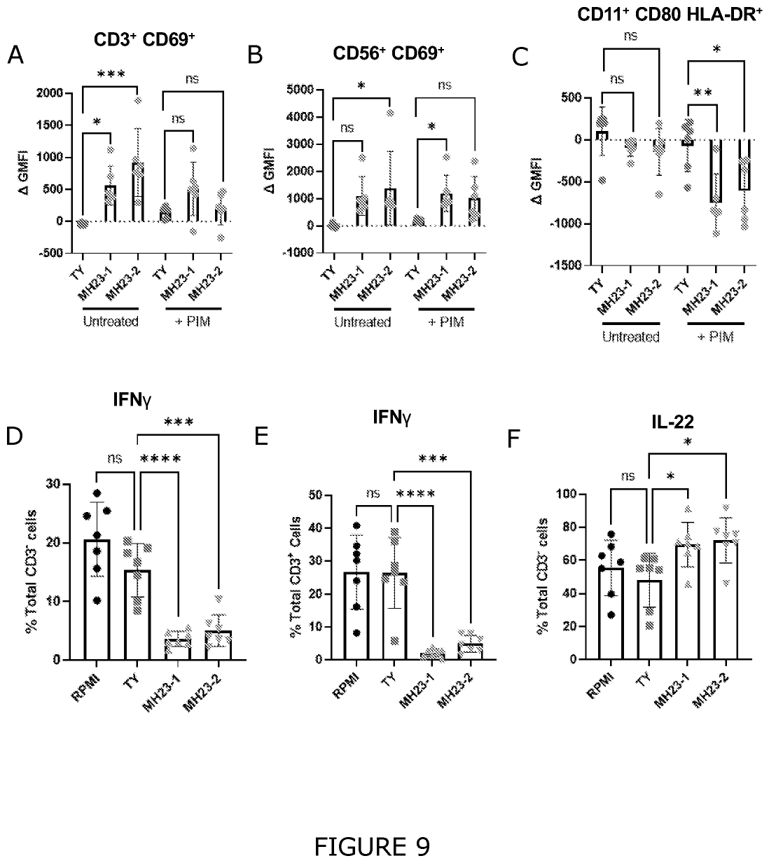

It was hypothesised that the bioactives produced by M. faecis may have direct effects on peripheral immune cells. In order to interrogate this hypothesis, first the ability of M. faecis culture supernatant to prevent IL-6 mediated activation of T cells (CD3⁺ CD4⁺, CD3⁺ CD8⁺, CD3⁺ TCRγδ⁺), Natural Killer Cells (CD56⁺) and Antigen Presenting Cells (CD11b⁺, CD11b⁺ CD80⁺, CD11c⁺, CD11c⁺ CD80⁺) was assessed. Treatment with M. faecis MH23 CS resulted in an increased geometric mean of the fluorescent intensity (GMFI) of CD69 expressing populations of the total CD3⁺ T cell populations and the CD3⁺ CD4⁺, CD3⁺ CD8⁺, but not CD3⁺ TCRγδ⁺ cell sub-populations relative to the TY medium control (FIG. 9A-C). There was also an increased GMFI of CD69 expressing populations of natural killer cells following treatment with M. faecis MH23-2 supernatant or M. faecis MH23-1 supernatant and PIM. In contrast, there was a modest reduction in GMFI of HLA-DR expressing populations of CD11b⁺ CD80⁺ HLA-DR⁺ cells (FIG. 9C), and CD11b⁺ (MH23-1 and MH23-2), CD11b⁺ CD80⁺ (MH23-1 and MH23-2), CD11c⁺ (MH23-2) and CD11c⁺ CD80⁺ (M. faecis MH23-1 and MH23-2) cells relative to the TY medium control (FIG. 9A-C).

Next, the effect of M. faecis CS on cytokine production in CD3⁺ and CD3⁻ cells derived from peripheral blood was assessed. CD3⁺ and CD3⁻ cells stimulated with PMA/ionomycin/monensin were characterised by a significant increase in IFNγ production. Treatment with M. faecis strain MH23-1 or MH23-2 CS inhibited IFNγ production to basal levels relative to the TY medium (FIG. 9D-E; p<0.01). Separately, treatment with M. faecis MH23-1 or MH23-2 culture supernatant induced IL-22 production relative to the TY medium (FIG. 9F) in CD3⁻ cells. Taken together, this shows that M. faecis can modulate key cytokines that are considered characteristic of Th1 and Th17 immune driven responses.

M. faecis Promotes Intestinal Epithelial Cell Migration.

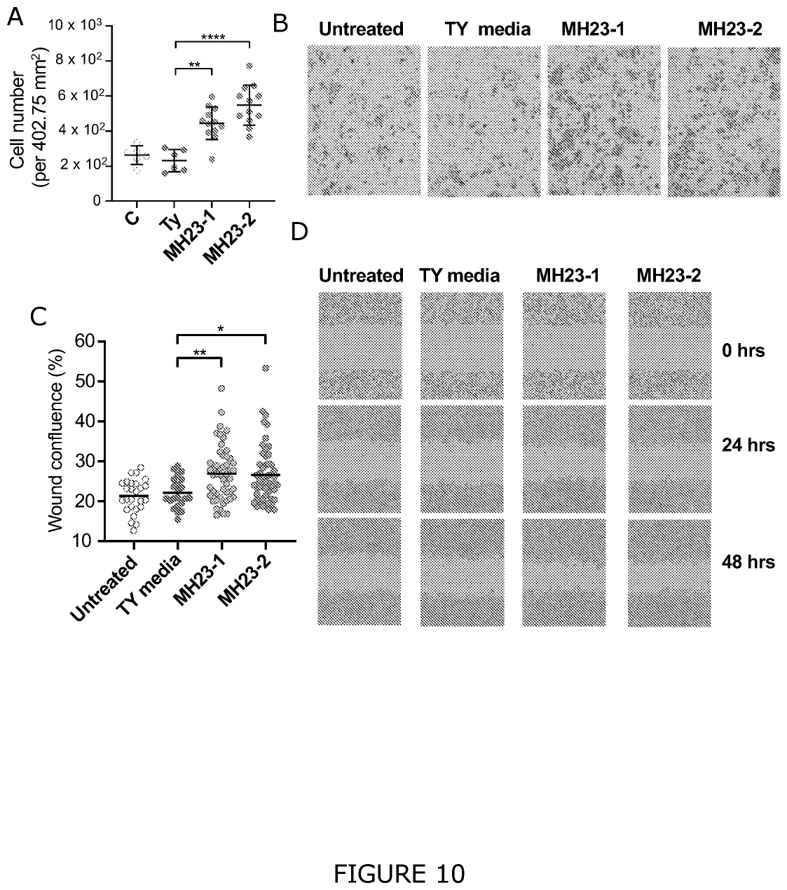

Damage of the intestinal barrier commonly occurs in inflammatory and autoimmune conditions. The rapid migration of intestinal epithelial cells is a crucial component of the wound healing process to re-establish homeostasis. To investigate whether bioactives secreted by M. faecis can affect the motility of intestinal epithelial cells, a Transwell® migration assay was employed. HCT116 cells were seeded apically in a Transwell® chamber and the ability of an M. faecis extract to promote migration to the basolateral side of the chamber was assessed. DMEM-treated cells had a basal level of cell migration and this was unaffected by treatment with TY medium (FIG. 10A,B). Notably, treatment with M. faecis MH23-1 and MH23-2 both significantly promoted the migration of HCT116 cells (FIG. 10A,B).

The pro-migratory effects of M. faecis were further confirmed using the IncuCyte scratch wound assay. After induction of a scratch wound, HCT116 cells showed an accelerated rate of wound closure in the presence of extract from M. faecis strains MH23-1 and MH23-2, compared to the control cells treated with TY medium (FIG. 10C,D).

M. lactaris Suppresses IL-23-Mediated STAT3 Activation.

In light of the above results, the inventors next sought to confirm that these results could be extrapolated to highly similar bacterial species that were also associated with inflammatory and autoimmune disease. In this regard, the ability of the culture supernatant of two M. lactaris species (M. lactaris ATCC 29176 and M. lactaris MH54) to suppress IL-23-mediated activation of STAT3 was assessed using the HEK Blue IL-23 reporter cell line. The HEK Blue IL-23 cell line contains a STAT3 responsive SEAP reporter whose expression is induced by IL-23. Treatment with tofacitinib, a pan JAK inhibitor, inhibits IL-23 mediated SEAP expression. Treatment with TY medium resulted in modest suppression of IL-23 mediated activation of STAT3 (FIG. 11). The present inventors examined the ability of culture supernatant and the <3 kDa supernatant fraction to modulate STAT3 activity. The <3 kDa fraction was assessed as it was hypothesized that the suppressive effect would be due to low molecular weight bioactives, which would have the advantage of being more amenable to drug development.

Both M. lactaris strains suppressed IL-23-mediated STAT3 activation (FIG. 1A-B). Notably, the M. lactaris <3 kDa fractions suppressed IL-23-mediated STAT3 activation to a similar extent as the cell free culture supernatant.

M. faecis Supports Gut Barrier Function and Improves Gut Barrier Integrity.

Intestinal epithelial cells form a physical and biochemical barrier that separates host tissue from gut microbes and luminal contents (Peterson and Artis, 2014). Impaired gut barrier function has been implicated in the pathogenesis of several diseases including IBD (Vanuytsel et al., 2021) and has been proposed as a therapeutic target to improve disease outcomes (Sommer et al., 2021). The integrity of the gut epithelial cell barrier can be assessed using a simple, non-invasive method termed trans-epithelial electrical resistance (TEER). In the TEER assay, an electrical current is applied across an epithelial cell layer and the resistance is measured. A reduction in the TEER value is indicative of a compromised barrier. TEER measurements constitute the “gold standard” for non-invasive measurements of barrier integrity in mono-culture cell layers.

The present inventors assessed the ability of M. faecis to modulate barrier function using T84 gut epithelial cell line. Following treatment with IFN-γ for 24 hours there was a significant drop in resistance indicating an increase in barrier permeability. As expected, treatment with tofacitinib (see, Sayoc-Becerra et al., 2020) significantly ameliorated the reduction in TEER. Treatment with <3 kDa fractionated culture supernatant from M. faecis strain MH23-1 or MH23-3 also ameliorated the reduction in TEER relative to the TY medium control (FIG. 12A). At 144 hours post IFN-γ treatment, treatment with the culture supernatant M. faecis strain MH23-1 or MH23-3 ameliorated the reduction in TEER relative to the TY medium control (FIG. 12B).

The present inventors next assessed the ability of MH23 extract to promote restoration of barrier integrity following IFNγ treatment. Treatment with IFNγ for 72 hours resulted in a significant reduction in TEER. Consistent with previous reports (Boivin et al., 2009), the NF-κB inhibitor PDTC ameliorated the impact of the IFNγ treatment on TEER. Treatment with the YG/V medium extract did not affect TEER however MH23-3 culture supernatant extract resulted in a significant increase in TEER relative to YG/V control, which was indicative of improved barrier integrity (FIG. 12C). Taken together, these data demonstrate that M. faecis produces low molecular weight components that support maintenance and restoration of gut barrier integrity.

### Identification of Metabolites

The present inventors hypothesized that M. faecis produces metabolites that contribute to therapeutic efficacy. We identified 22 metabolites (classified as Level 1 or 2a) in the cell free culture supernatant that were 2-fold increase relative to the YG/V medium control (Table 5). These included metabolites previously shown to modulate inflammation, immune cell infiltration, oxidative stress and gut barrier function (e.g., ornithine (see, Qi et al., 2019) (1), N-acetyl-cysteine (e.g. (Masnadi Shirazi et al., 2021, and You et al., 2009) pyrogallol (Chicas et al., 2020) and propionylcarnitine (Scioli et al., 2014), amongst others).

TABLE 5
Identification of metabolites in supernatant

| Name | Fold change |
|---|---|
| Ornithine | 2806.953702 |
| N-Acetyl-glutamic acid | 272.2521184 |
| N8-Acetylspermidine | 113.9090955 |
| NAD+ | 53.59550359 |
| N-Acetyl-cysteine | 27.95733379 |
| Creatinine | 15.82069695 |
| Allopurinol | 10.81125942 |
| Inosine | 9.58598181 |
| delta-decenolactone | 9.004221142 |
| δ-Gluconolactone | 6.313585578 |
| Homocysteine | 5.314445862 |
| Acetylhistidine | 5.202521545 |
| Carbamoylaspartate | 5.190828821 |
| Pyrogallol | 4.091891298 |
| Adenine | 3.260166157 |
| Xanthosine | 3.112303737 |
| Propionylcarnitine | 2.947440406 |
| 4-Methyl-2-oxovaleric acid | 2.709985519 |
| Crotonic acid | 2.40415397 |
| 4-Hydroxy-2,5-dimethyl- | 2.289388196 |
| 3(2H)-furanone | |
| Uridine | 2.109979308 |
| Riboflavin | 2.100136471 |

### Materials & Methods

### Bacterial Strains, Culture Conditions and Analyses.

Stool samples were collected from healthy human adults with no history of gastrointestinal disorders and mixed with an equal weight per volume of sterile oxygen free glycerol solution (McSweeney et al., 2005). Donors had not consumed antibiotics in the three months prior to collection of the faecal samples. M. faecis and Faecalibacterium prausnitzii were routinely processed in a Coy vinyl anaerobic chamber with an oxygen free atmosphere (85% N₂:10% CO₂:5% H₂) atmosphere. M. faecis was routinely cultured in TY or PYG medium (as described in McSweeney et al., 2005) while F. prausnitzii was cultured in TY medium (McSweeney, 2005). All isolates were stocked by mixing 3 mL of actively growing culture with an equal volume of glycerol solution and storing at −80° C.

Isolation of M. faecis.

Enrichments of M. faecis MH23-1 and MH23-2 were produced by inoculating a donor faecal sample with M. faecis present at a relative abundance of 0.47% into Schaedler broth and then serially diluting to extinction. The dilution-to-extinction culture series was sequenced and an enrichment culture with M. faecis at 14% relative abundance was identified. This enrichment was subsequently diluted to extinction in Schaedler broth and an enrichment in the culture series with M. faecis at 42% relative abundance was identified. Colonies were recovered by streaking on Bacteroides Bile Esculin Agar with the aminoglycoside antibiotics omitted and two isolates termed M. faecis MH23-1 and MH23-2 were identified by whole genome sequencing. M. faecis MH23-3 was produced by inoculating a donor faecal sample with M. faecis at 0.12% relative abundance into Yeast N-acetylglucosamine broth (the recipe for Yeast N-acetylglucosamine broth is as per YG medium in Table 1 except that glucose is replaced by an equal amount of N-acetylglucosamine) and then serially diluting to extinction. An enrichment with M. faecis at 47.8% was identified and axenic isolates were subsequently produced by streaking on TY medium supplemented with 0.5% v/v sodium azide solution (10% w/v). M. faecis MH23-3 was identified by whole genome sequencing. M. faecis MH23-4 was produced by inoculating a donor faecal sample with M. faecis at 0.37% relative abundance into Yeast Fructose (YF) broth (the recipe for YF broth is as per YG medium in Table 1 except that glucose is replaced by an equal amount of fructose) and then serially diluting to extinction. An enrichment with M. faecis at 94.8% was identified and axenic isolates were subsequently produced by streaking on TY medium. M. faecis MH23-4 was identified by whole genome sequencing.

Isolation of M. lactaris.

M. lactaris (strain 14.1.C2, MH54) was isolated using from a faecal sample from a healthy subject with no history of gastrointestinal disorders. M. lactaris and M. torques were routinely processed in a Coy vinyl anaerobic chamber with an oxygen free atmosphere (85% N₂:10% CO₂:5% H₂) atmosphere. M. lactaris and M. torques were routinely cultured using TY or YG/V medium (Table 1). M. lactaris MH54 was isolated on TY agar. All isolates were stocked by mixing 3 mL of actively growing culture with an equal volume of glycerol solution and stored at −80° C.

### Metabolic Reconstruction.

Protein coding sequences were predicted and annotated using the annotate function in enrichM (version 0.5.2). Briefly, enrichM identifies protein coding sequences using prodigal (version 2.6.3) in -p meta mode. The amino acid sequences are then searched against the UniRef100 database (downloaded November 2020) using DIAMOND (version 2.0.4), and E.C., TCDB and eggnog classifications are inherited from the idmapping file distributed with UniRef. Hmmer hmmsearches (version 3.1b2) against Pfam (release 33.0), tigrfam (release 15.0) and dbcan2 (downloaded September 2019) were used to annotate functional domains, key metabolic markers and carbohydrate activate (CAZy) enymes, respectively. Metabolic pathways were identified using the classify function in enrichM, which assesses annotations and their genomic position against manually defined metabolic pathway definitions. A pathway is considered present in a genome if it encoded >80% of the required proteins, and passes all required synteny checks. These automatically predicted pathways were then manually assessed. In addition, gutSMASH (version 1.0.0) was applied to identify common biosynthetic pathways encoded by gut microorganisms.

### Phylogenetic Tree

A genome tree was constructed from high quality genomes, defined as ≥90% complete and ≤−5% contamination from checkM analysis, within the Mediterraneibacter genus (NCBI r95) and the four MH23 isolates. For each genome, a set of 122 bacteria-specific conserved marker genes were extracted from each genome using gtdbtk identify. These genes were the aligned to profile HMMs and concatenated to a single alignment with gtdbtk align, and a Maximum likelihood phylogenetic tree was constructed from the alignments using FastTree (version 2.1.10) with gtdbtk infer. Non-parametric bootstrap values were inferred using GenomeTreetk (v0.1.6) from 1000 repetitions.

### Preparation of Bacterial Strains for Animal Experimentation.

M. faecis and F. prausnitzii strains were grown to early stationary phase in TY medium. The cell density of the individual cultures was calculated using a Helber Counting Chamber. To prepare the bacterial gavage solutions, individual cultures were centrifuged under a layer of sterile heavy mineral oil at 5,000 g for 10 minutes and the cell-free supernatant was then discarded. The cell pellets were washed in 1.5 mL of sterile anaerobic buffered diluent (38 mL/L each of salt solutions 2 & 3 (McSweeney et al., 2005), 1 mL/L of 0.1% (w/v) resazurin solution, 1 g/L L-cysteine) and then centrifuged again. Finally, the washed cell pellet was resuspended in half strength glycerol solution (15% v/v glycerol solution in anoxic buffered diluent) to a final concentration of 1×10⁹ cells/mL, aliquoted and frozen at −80° C. until required. The viability of the cell preparations was confirmed by thawing a single aliquot and streaking on an agar plate. The identity and purity of the individual strain preparations was confirmed by whole genome sequencing.

### Acute Model of DSS-Induced Gut Barrier Dysfunction.

Six-week-old C57BL/6 female mice purchased from Animal Resources Centres (Western Australia, Australia) were randomised and then co-housed for seven days prior to experimentation. To induce gut barrier dysfunction, mice were treated with 3% DSS ad libiteum in the drinking water for six days. Naïve age matched control mice were processed and received DSS free drinking water. All treatments started one day prior to provision of DSS and all mice were sacrificed two days after the final DSS treatment. For the treatments, mice were anesthetised with isofluorane and orally gavaged with 200 μl of bacterial preparations or vehicle control. Prednisone (2 mg/kg) was administered following anesthetisation by intraperitoneal injection. Body weights and stool consistency were recorded daily. Stool samples were collected daily. Following sacrifice, the colon, liver and spleen were collected for analysis. Blood was collected by cardiac puncture. In the therapeutic model, six-week-old C57BL/6 female mice purchased from Animal Resources Centres (Western Australia, Australia) were randomised and then co-housed for seven days prior to experimentation. To induce gut barrier dysfunction, mice were treated with 2.5% DSS ad libiteum in the drinking water for six days. Naïve age matched control mice were processed and received DSS free drinking water. All treatments started two days prior to completion of the DSS treatments and all mice were sacrificed five days after the final DSS treatment. For the treatments, mice were anesthetised with isofluorane and orally gavaged with 200 μl of bacterial preparations or vehicle control. Prednisone (2 mg/kg) was administered following anesthetisation by intraperitoneal injection. Stool samples were collected daily and following sacrifice, the colons were collected for analysis.

### Endoscopic and Histological Scoring.

Animals were examined with a small animal gastrointestinal endoscope (Karl Storz Endoskope, Tuttlingen, Germany) on days 1, 2 and 6 to assess the extent of colon mucosal inflammation (Marks et al., 2015; and Liu et al., 2019). Briefly, mice were anesthetised with isoflurane and a colonoscope was inserted through the rectum. Images captured by high-definition videos were examined in a blinded manner to assess the presence and extent of disease pathologies (Table 6). Histological scoring was performed essentially as previously described in the art (see, Marks et al., 2015). Briefly, samples were fixed in 4% formalin, paraffin embedded and sectioned. Tissue sections were hematoxylin and eosin stained to assess disease pathology and with Alcian blue to assess mucin production. Slides were imaged using the Aperio digital imaging system (Leica Biosystems, NuBloch, Germany). To grade colitis severity, the extent of inflammation (colitis activity, see Table 6) and epithelial injury (composite of epithelial hyperplasia and injury, see Table 6) in the tissue sections were graded semi-quantitatively using an established scoring system (Table 7). The samples were then randomised and subsequently scored in a blinded by a trained gastrointestinal pathologist.

TABLE 6
Scoring of Gut Barrier Dysfunction by Colonoscopy

| | |
|---|---|
| Feature | Scoring |

Thickening of the colon wall

| | |
|---|---|
| Transparent | 0 |
| Moderate | 1 |
| Marked | 2 |
| Intransparent | 3 |

Changes in vascular pattern

| | |
|---|---|
| Normal | 0 |
| Moderate | 1 |
| Marked | 2 |
| Bleeding | 3 |

Granularity of the mucosal surface

| | |
|---|---|
| None | 0 |
| Moderate | 1 |
| Marked | 2 |
| Extreme | 3 |

Stool consistency/mucus secretion

| | |
|---|---|
| Normal and solid | 0 |
| Still shaped, mild mucus | 1 |
| Unshaped, mucus | 2 |
| Spread | 3 |

Extent of involved area

| | |
|---|---|
| 0-5% (none) | 0 |
| 5-20% (patchy) | 1 |
| 20-50% (moderate) | 2 |
| >50% (predominant) | 3 |

TABLE 7
SCORING OF HISTOLOGICAL COLITIS
Inflammation score (Scored 0-4)

| | |
|---|---|
| 0 | No evidence of inflammation |
| 1 | Low level of inflammation with scattered |
| | infiltrating mononuclear cells (1-2 foci |
| | only) |
| 2 | Moderate inflammation with multiple foci |
| 3 | High level of inflammation with increased |
| | vascular density and marked wall thickening |
| 4 | Maximal severity of inflammation with |
| | transmural leukocyte infiltration and |
| | loss of goblet cells |

Injury score (Scored 0-3)

| | |
|---|---|
| 0 | No epithelial injury |
| 1 | Occasional epithelial leison |
| 2 | 1-2 foci of ulceration |
| 3 | Extensive ulceration |

Colitis activity (Composite score (/15) based on measures listed below)

| | |
|---|---|
| Hypervascularisation | 0-3 based on severity |
| Presence of mononuclear | 0-3 based on severity |
| cells | |
| Epithelial hyperplasia | 0-3 based on severity |
| Epithelial injury | 0-3 based on severity |
| Presence of neutrophils | 0-3 based on severity |
| Lymphoid aggregates | Scored 0-2, where 0 = 0, 1 = ≤2, and 2 = ≥2 |

### TNBS Model of Colitis

Groups of 5 or 10 male BALB/c mice were used. Mice were fasted overnight (Day 1) before 2,4,6-Trinitrobenzene sulfonic acid solution (TNBS challenge on Day 2. Distal colitis was induced by intracolonic instillation of TNBS (1 mg in 0.1 mL 50% ethanol) after which, animals were kept in a vertical position for 30 seconds to ensure that the solution remained in the colon. For the treatments, mice were orally gavaged with 200 μl of M. faecis MH23-3 at 1×10⁹ cells/day or vehicle (sterile glycerol and phosphate salt solution) starting from Day 1 (i.e., 1 day before TNBS) to Day 5 for a total of 5 consecutive days (Days 1-5). The positive control Cyclosporin A was given at 75 mg/kg by oral gavage once daily from Day 1 (i.e., 1 day before TNBS) to Day 4 for a total of 4 consecutive days (Days 1-4). On the Day of TNBS challenge, vehicle, M. faecis MH-23.3 and Cyclosporine A were given at 2 hours before TNBS. Animals were sacrificed on Day 5; blood was collected from all animals by cardiac puncture. The colon tissue was also harvested and snap frozen with liquid nitrogen for cytokine measurement. On Day 5, the mice were euthanized by CO₂ asphyxiation. Each colon was removed, rinsed, and then cut from 4 cm from the anus. The tissue sections were fixed in 10% formalin and kept in 70% ethanol for histopathology.

### Histopathological Scoring of TNBS Colitis

Four-micrometre tissue sections were cut and stained with hematoxylin and eosin (H&E) for histological analysis (Colitis scoring; essentially as described by Dieleman L A et al., 1998) under light microscope (LEICA DM2700 M, USA). Histological criteria included: abnormalities of mucosal architecture, extent of inflammation, erosion or ulceration, epithelial regeneration, and the percentage involvement by the disease process. The scoring was based on the findings of observers by examining two sections from each colon per animal. Total score for colitis (Total Colitis Index) were added, resulting in a combined histologic score range from 0 to 40:

| | | |
|---|---|---|
| Abnormalities | None (normal) | 0 |
| of mucosal | Minimal for focal, not exceeding lamina propria | 1 |
| architecture | Mild abnormality, cystic dilation/aberrant crypts | 2 |
| | Moderate or multifocal abnormalities | 3 |
| | Severe, entire crypt and epithelium lost | 4 |
| Extent of | None | 0 |
| inflammation | Minimal for focal, scattered cells (<10%) | 1 |
| | Mild (10-25%) | 2 |
| | Moderate, inflammatory cells extending into the | 3 |
| | submucosa | |
| | Severe, transmural leukocyte infiltrate from | 4 |
| | mucosa to serosa | |
| Erosion or | No erosion, ulceration, or granulation tissue | 0 |
| ulceration | Minimal or focal, not exceeding lamina propria | 1 |
| | Unequivocal erosion | 2 |
| | Moderate, ulceration | 3 |
| | Severe, ulceration or granulation tissue | 4 |
| Epithelial | Complete regeneration or normal tissue | 0 |
| regeneration | Almost complete regeneration | 1 |
| | Regeneration with crypt depletion | 2 |
| | Surface epithelium not intact | 3 |
| | No tissue repair | 4 |
| Percent | None | 0 |
| involvement | 1-25% | 1 |
| | 26-50% | 2 |
| | 51-75% | 3 |
| | 76-100% | 4 |

Score between 0-20 for 2× sections and add scores for a total range 0-40.

### Characterisation of STAT3 Suppressive Activity.

To assess STAT3 suppressive activity, three independent colonies were inoculated and grown until early stationary phase. Then, each seed culture broth was used to inoculate two technical replicates each generating six technical replicates from three biological replicates. The technical replicates were grown until early stationary phase and then cell free culture supernatant was harvested as previously described (Giri et al., 2019). Culture supernatants were size fractionated by passing through a 3 kDa CENTRICON® column according to the manufacturer's (Merck Millipore) instructions.

STAT3 activity was assessed using the HEK Blue IL-23 cell line (Invivogen). Briefly, 50,000 cells per well were seeded in triplicate in a 96-well plate 24 hours prior to the start of the assay. Bacteria supernatant or sterile bacterial medium were mixed at a final concentration of 10%, 25%, or 50% v/v with recombinant human IL-23 (rhIL-23, R&D systems) at a final concentration of 5 ng/mL. This mixture was then added directly to the cells and incubated at 37° C. for 6 hours. The ability of the supernatants to suppress STAT3 activation was compared to the Janus kinase inhibitor tofacitinib (10 μM). STAT3 regulated SEAP reporter activity was assessed using Quanti Blue solution as recommended by the manufacturer (Invivogen). Results are the average of at least three independent experiments. Cytotoxicity was assessed using MTT assay. Briefly, MTT was added to the cells at a final concentration of 1.2 mM. The cells were incubated at 37° C. for 4 hours and then fixed with DMSO. Cytotoxicity was assessed by measuring absorbance at 540 nm as recommended by the manufacturer (Invitrogen, Thermo Fisher, Australia).

### Cytokine Production Assay.

Cytokine production was assessed using an Ella system (R&D Systems) or by using an IL-8 Human Uncoated ELISA Kit (Thermo Fisher, Australia). For the Ella based experiments, 1×10⁴ cells/well of THP-1 cells were seeded in 96-well plates. Following a 24 hour incubation, THP-1 were induced with PMA at the final concentration of 20 μM for 24 hours to differentiate them into macrophages. Differentiated THP-1 cells were treated with LPS (1 μM) and sample as appropriate and incubated at 37° C. for 6 hours and 24 hours. At these time points, cell supernatants were collected and analysed on the Ella system according to the manufacturer's instructions.

For the ELISA assays, 1×10⁴ cells/well of HCT116 cells were seeded in duplicate 96 well plates. HCT116 cells were treated with IL-1β (10 ng) and indole-3-carbinol (5 μM) or culture supernatant (10% v/v) as appropriate and incubated at 37° C. for 16 hours. Then, cell supernatants were collected and analysed using an IL-8 human uncoated ELISA Kit according to the manufacturer's instructions.

### GPCR and Cell Pathway Immunomodulatory Activity.

The ability of gut bacteria to modulate GPCR activity was assessed essentially as described by Colosimo et al., (Colosimo et al., 2019). Briefly, a single colony was inoculated and grown until early stationary phase. This “seed culture” broth was used to inoculate 600 mL of TY broth and the culture was incubated until early stationary phase. Culture supernatants were prepared by centrifuging the culture at 4000 g for 30 minutes and then passing the cell free supernatant through a 3 kDa filter according to the manufacturer's instructions (Sartorius Vivaflow® 50 Ultrafiltration Unit 3 kDa MWCO PES). Activated Amberlite XAD-7 resin was added to 400 mL of 3 kDa filtered cell-free supernatant (10% w/v), and the slurry was gently shaken overnight at 4° C. The resin was collected, washed with 400 mL of deionized water and then mixed with 120 mL of 100% methanol. Following a 2 hour incubation with gentle shaking, the methanol elution was collected. A second elution in 120 mL of 100% methanol was performed as previously described and the two elutions were ultimately combined and dried under vacuum using a rotary evaporator. The extract was fully resuspended at in 100% DMSO (thereafter referred to as 1000×) and stored at −20° C.

The GPCR modulatory activity of the M. faecis preparations was assessed using the gpcrMAX℠ GPCR Assay Panel (Eurofins, USA) in agonist and antagonist mode. Agonist and antagonist high confident hits were identified as described by Eurofins. Agonist and antagonist medium confident hits were identified as described by Eurofins except that except that the % activity/inhibition was ≥10%. For agonist mode, cells were incubated with supernatant extract to induce a response. Agonist activity was calculated using the following formula: percent activity=100%×(mean RLU of test sample−mean RLU of vehicle control)/(mean MAX control ligand −mean RLU of vehicle control). For the antagonist mode, cells were preincubated with supernatant extract and then treated with known GPCR specific agonists at the respective EC80 concentration. Antagonistic activity was calculated using the following formula: percent inhibition=100%×[1−(mean RLU of test sample−mean RLU of vehicle control)/(mean RLU of EC80 control−mean RLU of vehicle control)]. The SelectScreen™ Cell-based Pathway assays (Thermo Fisher, USA) were performed using supernatant extracts in agonist and antagonist mode. Activation in the activator assays was calculated using the following formula: percent activity=100%×(Response Ratio (supernatant)−Response Ratio (No activation control)/(Response Ratio (Full activation control)−Response Ratio (No activation control)). Inhibition in the inhibitor assays was calculated using the following formula: percent inhibition=100%×[1−(Response Ratio (supernatant)−Response Ratio (No activation control)/(Response Ratio (EC80 control)−Response Ratio (No activation control)). Agonist and antagonist hits were identified as described by the Thermo Fisher.

### Cell Migration Assay.

The IncuCyte® Live-Cell Imaging System (Essen BioScience) and transwell migration assay were used to assess the migration of HCT116 cells during exposure to sterile culture supernatant from M. faecis. Human HCT116 gut epithelial cells were maintained in McCoys 5a medium supplemented with 10% FBS and 1% Pen/Strep. For the IncuCyte® scratch wound assay, 3.5×10⁴ HCT116 cells were plated on poly-L-ornithine-coated IncuCyte® ImageLock 96-well plates (Essen BioScience). After 24 hours, the IncuCyte® WoundMaker tool was used to induce a homogeneous scratch wound in the nearly confluent cell monolayer. The cells were washed twice with DPBS and then stationary phase culture supernatant from M. faecis at 1% (v/v) in 200 μL 0.5% FBS McCoys 5a medium was added. Uninoculated bacterial media (1%) served as negative control. Immediately after adding the stimulants, the plate was transferred to the IncuCyte® system and cell migration was monitored by imaging each well every 2 hours over the course of 72 hours. Data analysis was performed using the integrated analysis software.

To assess cell migration via the TRANSWELL® assay, 3.5×10⁴ HCT116 cells were seeded in 100 μL 10% FBS culture medium in the top compartment of a 6.5 mm insert with TC-treated polycarbonate membrane in 24-well plates (8 μm pore size, Corning Costar). 600 μL 10% FBS culture medium was added to the lower compartment. The cells were allowed to settle for 24 hours. After a DPBS wash, 100 μL and 600 μL of 0.5% FBS medium was added to the top and lower compartment, respectively. Then, 0.4× concentrated extract from M. faecis was added to the lower compartment. These bacterial extracts were prepared using an Amberlite XAD-7 resin as previously described. After 16 hours, the cells were washed with DPBS and the cells attached to the top of the membrane were carefully removed with a cotton tip. The migrated cells on the bottom of the membrane were then fixed in 70% ethanol for 10 minutes followed by staining in 0.25% crystal violet for 5 minutes. The TRANSWELL® inserts were washed with water, dried and the membrane mounted with 50% glycerol in water on glass slides and imaged immediately. TRANSWELL® experiments were performed in biological and technical triplicates and for each replicate two representative images of the membrane were taken at 10× magnification. The number of migrated cells was automatically counted using ImageJ and the average cell number displayed. The extent of cell migration was expressed as the average number of migrated cells in two microscopic fields per well from three biological and three technical replicates.

### Characterisation of PBMC Immunomodulatory Activity.

PBMCs were extracted as previously described (Mallone et al., 2011). Briefly, PBMCs were isolated from blood samples using a Ficoll/Lymphoprep gradient (Stemcell), frozen at −80° C., and stored in liquid nitrogen. For the experiments, 250,000 PBMCs/well were seeded in 24-well plates. PBMCs were stimulated with IL-1β (50 ng/mL), IL-6 (10 ng/mL) for 14 hours at 37° C., and with PMA/Ionomycin/Monensin (40 ng/mL, 1 mg/mL, and 2 mg/mL respectively) for 4 hours at 37° C. An untreated control was used as negative control. Where indicated, PBMCs were pre-treated for 30 minutes at 37° C. with bacterial supernatant or bacterial medium control at final concentration of 10% v/v. PBMCs were stained for the following cell markers using the dilutions determined after performing antibody titration optimizations on unstimulated, untreated human PBMCs: CD3, CD4, CD8, CD69, TCRγδ, CD56, CD11b, CD11c, and CD80. PBMCs were finally analysed with Cytoflex S (Beckman Coulter) and data analysed with FloJo V10.

To characterise the cytokines present in PBMC culture supernatants, cell culture media supernatants from PBMCs that were stimulated as described above were collected and stored at −80° C. These supernatants were subsequently thawed and used in the LEGENDplex human inflammation panel I assay (BioLegend) to determine effects on cytokine production (IL-1β, IFN-α2, IFN-γ, TNF, MCP-1, IL-6, IL-8, IL-10, IL-12p70, IL-17A, IL-18, IL-23, and GM-CSF). Undiluted supernatants were incubated with capture beads conjugated to an antibody against specific analyte. After washing, a biotinylated detection antibody mixture is added to create a “capture bead-analyte-detection antibody” sandwich. Streptavidin-phycoerythrin (SA-PE) is added to bind to the biotinylated detection antibodies. After a final wash, the mixtures are analyzed by flow cytometry where the beads are differentiated by size and internal fluorescence intensities. Cytokine concentration is determined by PE fluorescence and comparison to a standard curve of known cytokine concentrations using BioLegend's LEGENDplex data analysis software. The unique size and fluorescence characteristics of the beads allows for the simultaneous measurement of 13 cytokines.

### Epithelial Barrier Integrity Assay Using Trans-Epithelial Electrical Resistance (TEER).

The ability of MH23 to prevent loss of barrier integrity was assessed by measuring the transepithelial electrical resistance across confluent monolayers of T84 gut epithelial cells. T84 cells were purchased from CellBank Australia and cultured in Dulbecco's Modified Eagle Medium Nutrient Mixture 12 (DMEM/F12; ThermoFisher Scientific, Waltham, MA, USA) supplemented with 5% foetal bovine serum and 1% Penicillin-Streptomycin. T84 cells were seeded into 24-well Millicell polycarbonate cell culture inserts with 0.4 μm pore size (PSHT010R5) at a density of 60.000 cells per well in 400 μl medium, and 24 ml of medium was added into the single-well feeder tray. Media changes of both compartments were performed every second day. After 7 days of culture, the upper part of the plate assembly with cell culture inserts were transferred from the feeder tray to a 24-well receiver tray (PSMW010R5), with each well containing 800 μl media. TEER values of each well were then measured daily using the Millicell ERS-2 Voltohmmeter. Experiments were started once the cells in all wells reached stable TEER readings above 1500Ω. Tofacitinib (75 μM), 3 kDa-filtered bacterial medium (TY) or 3 kDa-filtered MH23 bacterial supernatant diluted to 5%, 10% or 20% in T84 medium were added to the apical compartment. After one hour pre-treatment, IFNγ (50 ng/ml) was added basolateral to disrupt barrier integrity and removed after 48 hours treatment. Every 24 hours, treatments and medium were refreshed, and the TEER values were measured in duplicate. The data from two biological replicates with duplicate measurements each were presented as the percentage difference in TEER value compared to the control (untreated T84 cells). Statistical significance was determined by unpaired t-test.

The ability of MH23 to promote recovery of barrier integrity was also assessed by using the Maestro Pro system. Briefly, T84 cells were grown in (DMEM/F12) supplemented with 10% heat-inactivated foetal bovine serum and 1% Penicillin-Streptomycin in T75 flasks at 37° C., 5% C02 until approximately 75% confluent. Cells were seeded at a density of 0.1×10⁶ cells in a total of 0.2 mL/well in 96-well CytoView-Z plates and incubated at 37° C., 5% C02 in the Maestro Pro system (Axion BioSystems, Atlanta, GA, USA). Media was refreshed every three days. Once stabile TEER measurements had been established (approximately 100 h after seeding), cells were stimulated with recombinant human IFNγ (100 ng/mL; R&D systems, Minneapolis, MN, USA). At 72 h post-stimulation, cells were washed with dPBS (ThermoFisher Scientific, Waltham, MA, USA) and media was replaced with DMEM/F12 only, or DMEM/F12 supplemented with Pyrrolidine dithiocarbamate (PDTC; 50 μM), 1×YG/V media control or 1×MH23-3 bacterial culture supernatant extracts prepared using Amberlite XAD-7 resin as previously outlined. All conditions were analysed as quadruplicates. At 5 h post-treatment, TEER measurements were recorded, and the observed effect of treatment was presented as the percentage difference in TEER value compared to the control (untreated T84 cells). Statistical significance was determined by unpaired t-test.

### Metabolomic Analysis

To assess the metabolites produced by M. faecis MH23, six independent colonies were inoculated and grown until early stationary phase. Then, each seed culture broth was used to inoculate two technical replicates of YG/V generating 12 technical replicates from six biological replicates. The technical replicates were grown until early stationary phase and then cell free culture supernatant was harvested following centrifugation at 12,550 g for 3 minutes in an anaerobic chamber. Culture supernatants were snap frozen on dry ice and then stored at −80° C. until testing.

Sample analysis was carried out by MS-Omics (Denmark) as follows. The analysis was carried out using a Thermo Scientific Vanquish LC coupled to Thermo Q Exactive HF MS. An electrospray ionization interface was used as ionization source. Analysis was performed in negative and positive ionization mode. The UPLC was performed using a slightly modified version of the protocol described by Catalin et al. (UPLC/MS Monitoring of Water-Soluble Vitamin Bs in Cell Culture Media in Minutes, Water Application note 2011, 720004042en). Peak areas were extracted using Compound Discoverer 3.1 (Thermo Scientific). Identification of compounds were performed at four levels; Level 1: identification by retention times (compared against in-house authentic standards), accurate mass (with an accepted deviation of 3 ppm), and MS/MS spectra, Level 2a: identification by retention times (compared against in-house authentic standards), accurate mass (with an accepted deviation of 3 ppm). Level 2b: identification by accurate mass (with an accepted deviation of 3 ppm), and MS/MS spectra, Level 3: identification by accurate mass alone (with an accepted deviation of 3 ppm).

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### REFERENCES

Arumugam, M. et al., Enterotypes of the human gut microbiome. Nature 473 (7346): 174-180 (2011).
Azad et al., Probiotic supplementation during pregnancy or infancy for the prevention of asthma and wheeze:
systematic review and meta-analysis. BMJ. (2013) 347:f6471.
Balato et al., (2014) J. Eur. Acad. Dermatol. Venereol. 28(8): 1016-24.
Brestoff J. R. and Atris D., Commensal bacteria at the interface of host metabolism and the immune system. Nat. Immunol., 14: 676-684 (2013).
Britto S L, Krishna M, Kellermayer R. Weight loss is a sufficient and economical single outcome measure of murine dextran sulfate sodium colitis. FASEB bioAdvances. 2019; 1(8): 493-7.
Chan, W., Chen, A., Tiao, D., Selinger, C. & Leong, R. Medication adherence in inflammatory bowel disease. Intest. Res. 15, 434 (2017).
Chaumeil, P. A., Mussig, A. J., Hugenholtz, P., and Parks, D. H., GTDB-Tk: A Toolkit to Classify Genomes with the Genome Taxonomy Database. (2020) Bioinformatics 36 (6): 1925-27.
Cheluvappa et al., (2014) Clin. Exp. Immunol. 175(2): 316-22.
Chicas M C, Fang C, Talcott S, Talcott S. Microbial Metabolites of Gallotannins Suppress Inflammation in RAW 264.7 Macrophages Through the Modulation of the AMPK/NF-kb Axis. Current Developments in Nutrition. 2020; 4 (Supplement_2): 375.
Colosimo D A, Kohn J A, Luo P M, Piscotta F J, Han S M, Pickard A J, et al. Mapping interactions of microbial metabolites with human G-protein-coupled receptors. Cell Host & Microbe. 2019; 26(2): 273-82.e7.
D'Haens, G, Sandborn, W J, Feagan, B G, et al. A review of activity indices and efficacy end points for clinical trials of medical therapy in adults with ulcerative colitis. Gastroenterology. 2007; 132: 763-86.
Fabro et al., (2015) Immunobiology, 220(1): 124-35.
GBD 2017 Inflammatory Bowel Disease Collaborators, The global, regional, and national burden of inflammatory bowel disease in 195 countries and territories, 1990-2017: a systematic analysis for the Global Burden of Disease Study 2017; Lancet, 5(1): 17-30.
Geva-Zatorsky, N. et al. Mining the human gut microbiota for immunomodulatory organisms. Cell 168, 928-943 (2017).
Giri R, Hoedt E C, Shamsunnahar K, McGuckin M A, Morrison M, Capon R J, et al. Secreted microbial metabolites modulate gut immunity and inflammatory tone. bioRxiv. 2019:2019.12.16.861872.
Goldin B. R., Gorbach S. L. Clinical indications for probiotics: an overview. Clin Infect Dis. (2008) 46 Suppl 2: 596-100.
Handbook of Pharmaceutical Excipients, 2nd Edition, (1994), Edited by A Wade and P J Weller.
Hunter-Cevera, J. C., Maintaining Cultures for Biotechnology and Industry (1996) Academic Press.
Kabat, A. M., Srinivasan, N. & Maloy, K. J. Modulation of immune development and function by intestinal microbiota. Trends Immunol. 35, 507-517 (2014).
Kailasapathy et al. (2002) Curr. Issues Intest. Microbiol. 3(2):39-48.
Karagozian, R. & Burakoff, R. The role of mesalamine in the treatment of ulcerative colitis. Ther. Clin. Risk Manag. 3, 893 (2007).
Kim, J., S. et al., Ruminococcus faecis sp. nov., isolated from human faeces; J Microbiol. 49 (3), 487-491 (2011).
Kim et al., Mediterraneibacter butyricigenes sp. nov., a butyrate-producing bacterium isolated from human faeces, J. Microbio. 57(1): 38-44; (2019).
Kunsch C, Rosen C A. NF-kappa B subunit-specific regulation of the interleukin-8 promoter. Mol Cell Biol. 1993; 13(10): 6137-46.
Masnadi Shirazi K, Sotoudeh S, Masnadi Shirazi A, Moaddab S-Y, Nourpanah Z, Nikniaz Z. Effect of N-acetylcysteine on remission maintenance in patients with ulcerative colitis: A randomized, double-blind controlled clinical trial. Clinics and Research in Hepatology and Gastroenterology. 2021; 45(4): 101532.
Li, X., Atkinson, M. A. The role for gut permeability in the pathogenesis of type 1 diabetes—a solid or leaky concept? Pediatr. Diabetes 16, 485-492 (2015).
Liu G, Mateer S W, Hsu A, Goggins B J, Tay H, Mathe A, et al. Platelet activating factor receptor regulates colitis-induced pulmonary inflammation through the NLRP3 inflammasome. Mucosal Immunol. 2019; 12(4): 862-73.
Lloyd-Price J, Arze C, Ananthakrishnan A N, Schirmer M, Avila-Pacheco J, Poon T W, et al. Multi-omics of the gut microbial ecosystem in inflammatory bowel diseases. Nature. 2019; 569(7758): 655-62.
Mallone R., Mannering S. I., Brooks-Worrell B. M., Durinovic-Belló I., Cilio C. M., Wong F. S., et al. Isolation and preservation of peripheral blood mononuclear cells for analysis of islet antigen-reactive T cell responses: position statement of the T-Cell Workshop Committee of the Immunology of Diabetes Society. Clin Exp Immunol. 2011; 163(1): 33-49.
Marks E, Goggins B J, Cardona J, Cole S, Minahan K, Mateer S, et al. Oral delivery of prolyl hydroxylase inhibitor: AKB-4924 promotes localized mucosal healing in a mouse model of colitis. Inflamm Bowel Dis. 2015; 21(2): 267-75.
Masco, L. et al., Identification of Bifidobacterium species using rep-PCR fingerprinting, Systematic and Applied Microbiology, 26(4):557-563 (2003).
McSweeney C. S., Denman S. E., Mackie R. I., Rumen bacteria. In: Makkar HPS; Methods in gut microbial ecology for ruminants. 23-37 (2005).
Meddings, J. B., Jarand, J., Urbanski, S. J., Hardin, J., Gall, D. G. Increased gastrointestinal permeability is an early lesion in the spontaneously diabetic BB rat. Am. J. Physiol. 276, G951-G957 (1999).
Mitropoulou et al. (2013) J. Nutr. Metab. (2013) 716861,
Remington's Pharmaceutical Sciences, Mack Publishing Co. (A. R. Gennaro edit. 1985).
Monteleone et al., (2011) BMC Medicine. 2011, 9:122.
Oren A., et al., (2015). Proposal to include the rank of phylum in the international code of nomenclature or prokaryotes. Int. J. Syst. Evol. Microbiol., 65, 4284-4287.
Parks, D. H., Chuvochina, M., Waite, D. W., Rinke, C., Skarshewski, A., Chaumeil, P. A., and Hugenholtz, P. A Standardized Bacterial Taxonomy Based on Genome Phylogeny Substantially Revises the Tree of Life. (2018) Nature Biotechnology.
Peterson L. W., Artis D. Intestinal epithelial cells: regulators of barrier function and immune homeostasis. Nat Rev Immunol. 2014; 14:141.
Qi H, Li Y, Yun H, Zhang T, Huang Y, Zhou J, et al. Lactobacillus maintains healthy gut mucosa by producing L-Ornithine. Communications Biology. 2019; 2(1): 171.
Sapone, A. et al., Zonulin upregulation is associated with increased gut permeability in subjects with type 1 diabetes and their relatives. Diabetes 55, 1443-1449 (2006).
Sayoc-Becerra A., Krishnan M., Fan S., Jimenez J., Hernandez R., Gibson K., et al. The JAK-Inhibitor Tofacitinib Rescues Human Intestinal Epithelial Cells and Colonoids from Cytokine-Induced Barrier Dysfunction. Inflamm Bowel Dis. 2020; 26(3):407-22.
Schieck et al., (2014) J. Allergy Clin. Immunol. 133(3): 888-91.
Schindler et al., JAK-STAT signaling: from interfeors to cytokines, J. Biol. Chem. (2007) 282(28): 20059-63.
Schroeder, K W, Tremaine, W J, Ilstrup D M. Coated oral 5-aminosalicylic acid therapy for mildly to moderately activeulcerative colitis. A randomized study. N Engl J Med. 1987; 317: 1625-9.
Scioli M G, Stasi M A, Passeri D, Doldo E, Costanza G, Camerini R, et al. Propionyl-L-Carnitine is Efficacious in Ulcerative Colitis Through its Action on the Immune Function and Microvasculature. Clin Transl Gastroenterol. 2014; 5(3): e55-e.
Sokol H, Pigneur B, Watterlot L, Lakhdari O, Bermudez-Humaran L G, Gratadoux J J, et al. Faecalibacterium prausnitzii is an anti-inflammatory commensal bacterium identified by gut microbiota analysis of Crohn disease patients. Proc Natl Acad Sci USA. 2008; 105(43):16731-6.
Sommer K., Wiendl M., Müller T. M., Heidbreder K, Voskens C, Neurath M F, et al. Intestinal Mucosal Wound Healing and Barrier Integrity in IBD-Crosstalk and Trafficking of Cellular Players. Frontiers in Medicine. 2021; 8(295).
Sr\dot{u}tková, D. et al., Efficiency of PCR-based methods in discriminating Bifidobacterium longum ssp. Longum and Bifidobacterium longum ssp. infantis strains of human origin. J. Microbiol. Methods, (2011) 87(1): 10-6.
Togo A. H., et al., Description of Mediterraneibacter massiliensis, gen. nov., sp. nov., a new genus isolated from the gut microbiota of an obese patient and reclassification of Ruminococcus faecis, Ruminococcus lactaris, Ruminococcus torques, Ruminococcus gnavus and Clostridium glycyrrhizinilyticum as Mediterraneibacter faecis comb. nov., Mediterraneibacter lactaris comb. nov., Mediterraneibacter torques comb. nov., Mediterraneibacter gnavus comb. nov. and Mediterraneibacter glycyrrhizinilyticus comb. nov, Antonie Van Leeuwenhoek, 111(11): 2107-2128 (2018).
Toshimitsu T, Ozaki S, Mochizuki J, Furuichi K, Asami Y. Effects of Lactobacillus plantarum Strain OLL2712 Culture Conditions on the Anti-inflammatory Activities for Murine Immune Cells and Obese and Type 2 Diabetic Mice. Appl Environ Microbiol. 2017; (7): e03001-16.
Vanuytsel T., Tack J., Farre R. The Role of Intestinal Permeability in Gastrointestinal Disorders and Current Methods of Evaluation. Frontiers in Nutrition. 2021; 8.
Whitman, W. B., et al. (2018). Proposal of the suffix -ota do denote phyla. Addendum to “Proposal to include the rank of phylum in the International Code of Nomenclature or Prokayotes”. Int. J. Syst. Evol. Microbiol. 68, 967-969,
Yamamoto A, Itoh T, Nasu R, Nishida R. Effect of sodium alginate on dextran sulfate sodium- and 2,4,6-trinitrobenzene sulfonic acid-induced experimental colitis in mice. Pharmacology. 2013; 92(1-2): 108-16.
Ye et al., (2015) PLoS One, 10(1): e0117704.
Yin et al., (2014) Immunogenetics, 66(3): 215-8.
You Y, Fu J-J, Meng J, Huang G-D, Liu Y-H. Effect of N-acetylcysteine on the Murine Model of Colitis Induced by Dextran Sodium Sulfate Through Up-Regulating PON1 Activity. Digestive Diseases and Sciences. 2009; 54(8):1643-50.
Zhu Y, Yang S, Zhao N, Liu C, Zhang F, Guo Y, et al. CXCL8 chemokine in ulcerative colitis. Biomed Pharmacother. 2021; 138: 111427.

What is claimed is:

1. A cell of the Mediterraneibacter faecis strain deposited under any one of accession numbers V21/006223, V21/006224, V21/006225, or V21/006226, or a derivative thereof.

2. The cell of claim 1, wherein the cell is at least partially isolated.

3. A biologically pure culture of the Mediterraneibacter faecis strain deposited under any one of accession numbers V21/006223, V21/006224, V21/006225, or V21/006226, or a derivative thereof.

4. A composition comprising the cell of claim 1 or claim 2, or the culture of claim 3.

5. A composition comprising a bacterial strain with a 16S rRNA sequence that is at least about 97.5%, 98%, 98.5% 99%, 99.1%, 99.2%, 99.3%, 99.4%, 99.5%, 99.6%, 99.7%, 99.8%, or 99.9% identical to one or more of SEQ ID NOs: 1-24, or which has a 16S rRNA gene sequence represented by one or more of SEQ ID NOs: 1-24.

6. The composition of claim 4 or claim 5, further comprising a pharmaceutically acceptable excipient, diluent, or carrier.

7. A pharmaceutical composition comprising a bacterial strain with a 16S rRNA sequence that is at least about 97%, 98%, 99%, 99.5%, 99.6%, 99.7%, 99.8%, or 99.9% identical to the 16S rRNA sequence of a bacterial strain of the species Mediterraneibacter faecis, together with a pharmaceutically acceptable carrier, diluent, or excipient.

8. A pharmaceutical composition comprising a bacterial strain that is a phylogenetic descendant of the MRCA of M. faecis and M. lactaris, together with a pharmaceutically acceptable carrier, diluent, or excipient.

9. The pharmaceutical composition of claim 8, wherein the MRCA is defined at node 52630 of the bac120 phylogenetic tree from the Genome Taxonomy Database (GTDB).

10. The composition of any one of claims 5 to 9, wherein the bacterial strain is at least partially isolated.

11. The composition of any one of claims 4 to 9, wherein the bacterial strain is live or dead.

12. The composition of any one of claims 4 to 9, wherein the composition is in a dried form.

13. The composition of claim 12, wherein the composition is formulated in a capsule, a tablet, a pill, a troche, a lozenge, a powder, or a granule.

14. The composition of claim 12 or claim 13, wherein the composition is dried by lyophilization, spray drying, fluidized bed drying, vacuum drying, or a combination thereof.

15. The composition of any one of claims 4 to 14, wherein the composition is formulated for delivery to the gut.

16. The composition of any one of claims 4 to 15, further comprising a prebiotic.

17. The composition of any one of claims 4 to 16, further comprising one or more additional bacterial strains.

18. The composition of claim 17, wherein the one or more additional bacterial strains are at least partially isolated.

19. The composition of any one of claims 4 to 18, wherein the composition does not comprise bacteria of the genus Clostridium.

20. The cell, culture, or composition according to any one of claims 1 to 19, wherein the bacterial strain produces an agent that attenuates or impairs signal transducer and activator of transcription 3 (STAT3) signalling in a cell.

21. The cell, culture or composition according to claim 20, wherein the agent is a small molecule, peptide, or nucleotide.

22. The cell, culture or composition according to claim 20 or claim 21, wherein the agent is released by the bacteria.

23. The cell, culture or composition according to any one of claims 20 to 22, wherein the agent binds specifically to any one of STAT3, JAK2, TYK, or IL-23.

24. The cell, culture, or composition according to any one of claims 1 to 23, wherein the bacterial strain produces one or more metabolites selected from the group comprising or consisting of propionate, lactate, acetate, and formate.

25. The cell, culture, or composition according to any one of claims 1 to 24, wherein the bacterial strain does not produce butyrate.

26. The cell, culture, or composition according to any one of claims 1 to 25, wherein the bacterial strain produces vitamin B12.

27. The cell, culture or composition according to any one of claims 1 to 26, wherein the bacterial strain is of the species M. faecis.

28. The cell, culture or composition according to any one of claims 1 to 26, wherein the bacterial strain is of the species M. lactaris.

29. A food or drink product comprising the composition of any one of claims 4 to 28.

30. A method of restoring or improving gut barrier function in a subject, the method comprising administering to the subject a bacterial strain of the Mediterraneibacter genus, to thereby restore or improve gut barrier function.

31. The method of claim 30, wherein the bacterial strain is a phylogenetic descendant of the MRCA of M. faecis and M. lactaris.

32. The method of claim 31, wherein the MRCA is defined at node 52630 of the bac120 phylogenetic tree from r95 of the Genome Taxonomy Database (GTDB).

33. The method of any one of claims 30 to 32, wherein the bacterial strain is of the species M. faecis.

34. The method of any one of claims 30 to 32, wherein the bacterial strain is of the species M. lactaris.

35. The method of any one of claims 30 to 34, wherein restoring or improving gut barrier function is characterised by at least one of: (i) an increase in the quality and/or quantity of mucin; (ii) improvement in integrity of tight junction proteins; (iii) reduction in translocation of luminal contents into systemic circulation; or (iv) a reduction of intestinal ulcers and/or intestinal wounds.

36. The method of claim 35, wherein the luminal contents includes lipopolysaccharide (LPS).

37. The method of any one of claims 30 to 36, wherein the restoration or improvement in gut barrier dysfunction results in a reduction in systemic inflammation in the subject.

38. The method of claim 37, wherein systemic inflammation is identified in the subject when the level of an inflammatory cytokine (e.g., IL-1β, IL-8, IL-6, and TNF) in a sample from the subject is above a predetermined threshold.

39. A method of inducing or enhancing mucosal healing in a subject, the method comprising administering to the subject a bacterial strain of the Mediterraneibacter genus in an amount sufficient to induce epithelial cell migration and/or proliferation; to thereby induce mucosal healing in the subject.

40. The method of claim 39, wherein the bacterial strain is a phylogenetic descendant of the MRCA of M. faecis and M. lactaris.

41. The method of claim 40, wherein the MRCA is defined at node 52630 of the bac120 phylogenetic tree from r95 of the Genome Taxonomy Database (GTDB).

42. The method of any one of claims 39 to 41, wherein the bacterial strain is of the species M. faecis.

43. The method of any one of claims 39 to 41, wherein the bacterial strain is of the species M. lactaris.

44. The method of any one of claims 39 to 43, wherein mucosal healing is measured using one or more fecal or serum markers.

45. The method of claim 44, wherein one or more fecal markers are selected from the group comprising calprotectin, lactoferrin, metalloproteinase (MMP)-9, and lipocalin-2.

46. The method of any one of claims 39 to 45, wherein mucosal healing is measured using endoscopic score.

47. A method of reducing inflammation in a subject, the method comprising administering to the subject a therapeutically effective amount of a bacterial strain of the Mediterraneibacter genus, to thereby reduce inflammation in the subject.

48. The method of claim 47, wherein the bacterial species is a phylogenetic descendant of the MRCA of M. faecis and M. lactaris.

49. The method of claim 48, wherein the MRCA is defined at node 52630 of the bac120 phylogenetic tree from r95 of the Genome Taxonomy Database (GTDB).

50. The method of any one of claims 47 to 49, wherein the bacterial strain is of the species M. faecis.

51. The method of any one of claims 47 to 49, wherein the bacterial strain is of the species M. lactaris.

52. The method of any one of claims 47 to 51, wherein the inflammation is local to the gut environment, or systemic inflammation.

53. The method of any one of claims 47 to 52, wherein the bacterial strain attenuates the NFκB pathway (e.g., by reducing or inhibiting NFκB).

54. The method of any one of claims 47 to 53, wherein the bacterial strain inhibits production of one or more of cytokines or chemokines selected from the group comprising TNF, IFN-γ, IL-1β, IL-8, and MCP-1.

55. A method of blocking or otherwise inhibiting the activation of STAT3 signalling in a target cell, the method comprising contacting the target cell with at least a soluble component of a bacterial cell preparation of the Mediterraneibacter genus, to block or otherwise inhibit the activation of STAT3 signalling in the target cell.

56. The method of claim 55, wherein the bacterial strain is a phylogenetic descendant of the MRCA of M. faecis and M. lactaris.

57. The method of claim 56, wherein the MRCA is defined at node 52630 of the bac120 phylogenetic tree from r95 of the Genome Taxonomy Database (GTDB).

58. The method of any one of claims 55 to 57, wherein the bacterial strain is of the species M. faecis.

59. The method of any one of claims 55 to 57, wherein the bacterial strain is of the species M. lactaris.

60. The method of any one of claims 55 to 59, wherein the target cell is selected from the group comprising a reporter cell (e.g., a HEK cell), an immune cell (e.g., a Th17 immune cell), an epithelial cell, and an endothelial cell.

61. The method of any one of claims 55 to 60, wherein the bacterial cell preparation comprises a bacterial cell culture.

62. The method of claim 61, wherein the soluble component comprises the supernatant of the bacterial cell culture.

63. The method of any one of claims 55 to 62, wherein the soluble component is substantially depleted of bacterial cells.

64. The method of any one of claims 55 to 60, wherein the bacterial cell preparation comprises a bacterial cell pellet.

65. The method of claim 64, wherein the soluble component comprises soluble fraction of the lysed cells.

66. The method of claim 65, wherein the soluble fraction is substantially separated from the insoluble cell fraction by centrifugation.

67. The method of any one of claims 55 to 66, wherein the method is performed in vitro.

68. A method of blocking or otherwise inhibiting STAT3 signalling in the gut environment of a subject, the method comprising ad mistering to the subject a bacterial strain of the Mediterraneibacter genus, to block or otherwise inhibit STAT3 signalling in the gut environment of the subject.

69. The method of claim 68, wherein the bacterial strain is a phylogenetic descendant of the MRCA of M. faecis and M. lactaris.

70. The method of claim 69, wherein the MRCA is defined at node 52630 of the bac120 phylogenetic tree from r95 of the Genome Taxonomy Database (GTDB).

71. The method of any one of claims 68 to 70, wherein the bacterial strain is of the species M. faecis.

72. The method of any one of claims 68 to 70, wherein the bacterial strain is of the species M. lactaris.

73. The method of any one of claims 68 to 72, wherein the cell is an immune cell (e.g., a Th17 immune cell), epithelial cell, or endothelial cell.

74. The method of any one of claims 68 to 73, wherein the cell is an epithelial cell, wherein the bacterial strain or molecule increases the production of IL-22 in the subject.

75. The method of any one of claims 68 to 74, wherein the bacterial strain produces a molecule that is a direct inhibitor or an indirect inhibitor of STAT3.

76. The method of any one of claims 68 to 75, wherein the bacterial strain produces a molecule that directly inhibits at least one of an IL-23 polypeptide, a JAK2 polypeptide, a TYK2 polypeptide, or a STAT3 polypeptide.

77. The method of any one of claims 30 to 76, wherein the bacterial strain produces one or more of the following metabolites: propionate, lactate acetate, and formate.

78. The method of any one of claims 30 to 77, wherein the bacterial strain produces vitamin B12.

79. The method of any one of claims 30 to 78, wherein the bacterial strain does not produce butyrate.

80. The method of any one of claims 30 to 79, wherein the bacterial strain has a 16S rRNA sequence that is at least about 95%, 96%, 97%, 98%, 99%, 99.1%, 99.2%, 99.3%, 99.4%, 99.5%, 99.6%, 99.7%, 99.8%, or 99.9% identical to the 16S rRNA sequence of a bacterial strain of the species M. faecis; or when the bacterial strain has a 16S rRNA gene sequence of a bacterial strain of M. faecis.

81. The method of any one of claims 30 to 79, wherein the bacterial strain has a 16S rRNA sequence that is at least about 97.5%, 98.5%, 99%, 99.1%, 99.2%, 99.3%, 99.4%, 99.5%, 99.6%, 99.7%, 99.8%, or 99.9% identical to one or more of SEQ ID NOs: 1-24, or when the bacterial strain has the 16S rRNA gene sequence represented by one or more of SEQ ID NOs: 1-24.

82. The method of any one of claims 30 to 81, wherein the bacterial strain is the Mediterraneibacter faecis strain deposited under any one of accession numbers V21/006223, V21/006224, V21/006225, or V21/006226, or a derivative thereof

83. The method of any one of claims 30 to 83, wherein the bacterial strain is at least partially isolated.

84. The method of any one of claims 30 to 53, wherein the bacterial strain is formulated as a pharmaceutical composition, further comprising a pharmaceutically acceptable carrier, diluent or excipient.

85. The method of claim 84, wherein the composition is in a dried form.

86. The method of claim 85, wherein the dried form is selected from the group comprising particles, granules, and powder.

87. The method of claim 85 or claim 86, wherein the composition is dried by lyophilization, spray drying, fluidized bed drying, vacuum drying, or a combination thereof.

88. The method of any one of claims 84 to 87, wherein the pharmaceutical composition is formulated for oral administration.

89. A method of treating or preventing an inflammatory or autoimmune disorder in a subject, the method comprising administering an effective amount of a bacterial strain of the Mediterraneibacter genus to the subject, to thereby treat or prevent the inflammatory or autoimmune disorder.

90. The method of claim 89, wherein the bacterial strain is a phylogenetic descendant of the MRCA of M. faecis and M. lactaris.

91. The method of claim 90, wherein the MRCA is defined at node 52630 of the bac120 phylogenetic tree from r95 of the Genome Taxonomy Database (GTDB).

92. The method of any one of claims 89 to 91, wherein the bacterial strain is of the species M. faecis.

93. The method of any one of claims 89 to 91, wherein the bacterial strain is of the species M. lactaris.

94. The method of claim 93, wherein the inflammatory or autoimmune disorder is selected from the group comprising an inflammatory bowel disease (such as Crohn's disease or ulcerative colitis); asthma (such as allergic asthma or neutrophilic asthma); arthritis (such as rheumatoid arthritis, osteoarthritis, psoriatic arthritis, or juvenile idiopathic arthritis); fatty liver disease (such as nonalcoholic fatty liver disease (NAFLD)); ankylosing spondylitis; psoriasis; systemic lupus erythematosus (SLE); scleroderma; Sjogren's syndrome; vasculitis; type 1 diabetes mellitus.

95. The method of claim 89 or claim 94, wherein the inflammatory or autoimmune disorder is an inflammatory bowel disease (IBD).

96. The method of any one of claims 89 to 95, wherein when administered to a subject, the bacterial strain blocks or otherwise inhibits STAT3 signalling in at least a cell of the subject.

97. The method of claim 96 wherein the cell is an epithelial cell, immune cell (e.g., a Th17 immune cell), or an endothelial cell.

98. The method of any one of claims 89 to 97, wherein the bacterial strain produces one or more metabolites selected from the group comprising propionate, lactate, acetate, and formate.

99. The method according to any one of claims 89 to 98, wherein the bacterial strain does not produce butyrate.

100. The method according to any one of claims 89 to 99, wherein the bacterial strain produces vitamin B12.

101. The method according to any one of claims 89 to 100, wherein the bacterial strain has a 16S rRNA sequence that is at least about 95%, 96%, 97%, 98%, 99%, 99.5%, 99.9% identical to the 16S rRNA sequence of a bacterial strain of the genus Mediterraneibacter.

102. The method according to any one of claims 89 to 100, wherein the bacterial strain has a 16S rRNA sequence that is at least about 95%, 96%, 97%, 98%, 99%, 99.5%, or 99.9% identical to one or more of SEQ ID NOs: 1-24, or when the bacterial strain has the 16S rRNA gene sequence represented by one or more of SEQ ID NOs: 1-24.

103. The method according to any one of claims 89 to 102, wherein the bacterial strain is at least partially isolated.

104. The method according to any one of claims 89 to 103, wherein the bacterial strain is formulated as a pharmaceutical composition, together with a pharmaceutically acceptable carrier, diluent, and/or excipient.

105. The method of claim 104, wherein the composition is in a dried form.

106. The method of claim 105, wherein the dried form is selected from the group comprising particles, granules, and powder.

107. The method of claim 104 or claim 105, wherein the composition is dried by lyophilization, spray drying, fluidized bed drying, vacuum drying, or a combination thereof.

108. The method of any one of claims 89 to 107, wherein an anti-inflammatory agent is co-administered to the subject.

109. The method of claim 108, wherein the anti-inflammatory agent is selected from the group comprising a 5-aminosalicylate, corticosteroid, azathioprine, infliximab, and adalimumab.

110. The method of any one of claims 30 to 109, wherein treating comprises, prior to administering the composition to the subject, identifying that the subject has a deficiency in M. faecis gut bacteria.

111. The method of claim 110, wherein identifying the deficiency in the subject comprises measurement of M. faecis bacteria in the subject's stool by 16S rRNA sequencing and/or whole genome sequencing.

112. The composition of any one of claims 30 to 111, wherein the bacterial strain is live or dead.

113. The composition of any one of claims 30 to 112, further comprising a prebiotic.

114. The composition of any one of claims 30 to 113, further comprising one or more additional bacterial strains.

115. The method of any one of claims 30 to 114, wherein the subject is a mammalian subject.

116. The method of any one of claims 30 to 115, wherein the subject is a human subject.

117. A composition comprising a bacterial strain of the genus Mediterraneibacter, for use in therapy.

118. The composition of claim 117, wherein the bacterial strain is a phylogenetic descendant of the MRCA of M. faecis and M. lactaris.

119. The composition of claim 118, wherein the MRCA is defined at node 52630 of the bac120 phylogenetic tree from r95 of the Genome Taxonomy Database (GTDB).

120. A composition comprising a bacterial strain of Mediterraneibacter faecis, for use in therapy.

121. A composition comprising a bacterial strain of Mediterraneibacter lactaris, for use in therapy.

122. A composition comprising a bacterial strain of the genus Mediterraneibacter, for use in the treatment or prevention of an inflammatory or autoimmune disorder.

123. The composition of claim 122, wherein the bacterial strain is a phylogenetic descendant of the MRCA of M. faecis and M. lactaris.

124. The composition of claim 123, wherein the MRCA is defined at node 52630 of the bac120 phylogenetic tree from r95 of the Genome Taxonomy Database (GTDB).

125. A composition comprising a bacterial strain of Mediterraneibacter faecis, for use in the treatment or prevention of an inflammatory or autoimmune disorder.

126. A composition comprising a bacterial strain of Mediterraneibacter lactaris, for use in the treatment or prevention of an inflammatory or autoimmune disorder.

127. Use of a bacterial strain of the genus Mediterraneibacter in the manufacture of a medicament for the treatment of an inflammatory or autoimmune disorder.

128. The use of claim 127, wherein the bacterial strain is a phylogenetic descendant of the MRCA of M. faecis and M. lactaris.

129. The use of claim 128, wherein the MRCA is defined at node 52630 of the bac120 phylogenetic tree from r95 of the Genome Taxonomy Database (GTDB).

130. Use of a bacterial strain of Mediterraneibacter faecis in the manufacture of a medicament for the treatment of an inflammatory or autoimmune disorder.

131. Use of a bacterial strain of Mediterraneibacter lactaris in the manufacture of a medicament for the treatment of an inflammatory or autoimmune disorder.

132. The composition or use of any one of claims 122 to 131, wherein the inflammatory or autoimmune disorder is selected from an inflammatory bowel disease (such as Crohn's disease or ulcerative colitis); asthma (such as allergic asthma or neutrophilic asthma); arthritis (such as rheumatoid arthritis, osteoarthritis, psoriatic arthritis, or juvenile idiopathic arthritis); fatty liver disease (such as nonalcoholic fatty liver disease (NAFLD)); ankylosing spondylitis; psoriasis; systemic lupus erythematosus (SLE); scleroderma; Sjogren's syndrome; vasculitis; type 1 diabetes mellitus.

133. The composition or use of any one of claims 122 to 132, wherein the inflammatory or autoimmune disorder is an inflammatory bowel disease (e.g., Crohn's disease or ulcerative colitis).

134. The composition of any one of claims 122 to 133, wherein the bacterial strain is the Mediterraneibacter faecis strain deposited under any one of accession numbers V21/006223, V21/006224, V21/006225, or V21/006226, or a derivative thereof.

135. A composition for use in treating an inflammatory or autoimmune disorder, the composition comprises a bacterial strain of the Mediterraneibacter genus; and an anti-inflammatory agent.

136. The composition of claim 135, wherein the bacterial strain is a phylogenetic descendant of the MRCA of M. faecis and M. lactaris.

137. The composition of claim 136, wherein the MRCA is defined at node 52630 of the bac120 phylogenetic tree from r95 of the Genome Taxonomy Database (GTDB).

138. The composition of any one of claims 135 to 137, wherein the bacterial strain is of the species M. faecis.

139. The composition of any one of claims 135 to 137, wherein the bacterial strain is of the species M. lactaris.

140. The composition of claim any one of claims 135 to 139, wherein the anti-inflammatory agent is selected from the group comprising 5-aminosaliculates, corticosteroids, azathioprine, infliximab, and adalimumab.

141. A composition for use in treating an inflammatory or autoimmune disorder, the composition comprises a bacterial strain of the Mediterraneibacter genus; and a nutritional supplement.

142. The composition of claim 141, wherein the bacterial strain is a phylogenetic descendant of the MRCA of M. faecis and M. lactaris.

143. The composition of claim 142, wherein the MRCA is defined at node 52630 of the bac120 phylogenetic tree from r95 of the Genome Taxonomy Database (GTDB).

144. The composition of any one of claims 141 to 143, wherein the bacterial strain is of the species M. faecis.

145. The composition of any one of claims 141 to 143, wherein the bacterial strain is of the species M. lactaris.

146. A method of improving or maintaining health in a subject, the method comprising administering to the subject a composition comprising a bacterial strain of M. faecis; to thereby maintain or improve health in the subject.

147. The method of claim 146, further comprising administering to the subject a nutritional supplement.

148. A comestible or potable product comprising a bacterial strain of the Mediterraneibacter genus; and a nutritional supplement.

149. The product of claim 148, wherein the bacterial strain is a phylogenetic descendant of the MRCA of M. faecis and M. lactaris.

150. The product of claim 149, wherein the MRCA is defined at node 52630 of the bac120 phylogenetic tree from r95 of the Genome Taxonomy Database (GTDB).

151. The product of any one of claims 148 to 150, wherein the bacterial strain is of the species M. faecis.

152. The product of any one of claims 148 to 150, wherein the bacterial strain is of the species M. lactaris.

153. The product of any one of claims 148 to 152, wherein the nutritional supplement is a prebiotic.
